SEQUENCE LISTING (53 sequences)

SEQ ID NO: 1 - DNA, 1820 bp, Homo sapiens
ttcaagtctttttcttttaacggattgatcttttgctagatagagacaaaatatcagtgtgaattacagcaaacccctattccatgctgttatgggtgaaactctgggagattctcctattgacccagaaagcgattccttcactgatacactgtctgcaaacatatcacaagaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactagcagagagtcctgagccactgccaacatttcccttcttccagttgcactattctgagggaaaatctgacacctaagaaatttactgtgaaaaagcattttaaaaagaaaaggttttagaatatgatctattttatgcatattgtttataaagacacatttacaatttacttttaatattaaaaattaccatattatgaaattgctgatagta

SEQ ID NO: 2 - protein, 505 aa, Homo sapiens
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 3 - DNA, 1837 bp, Homo sapiens
gagccgcgcacgggactgggaaggggacccacccgagggtccagccaccagccccctcactaatagcggccaccccggcagcggcggcagcagcagcagcgacgcagcggcgacagctcagagcagggaggccgcgccacctgcgggccggccggagcgggcagccccaggccccctccccgggcacccgcgttcatgcaacgcctggtggcctgggacccagcatgtctccccctgccgccgccgccgcctgcctttaaatccatggaagtggccaacttctactacgaggcggactgcttggctgctgcgtacggcggcaaggcggcccccgcggcgccccccgcggccagacccgggccgcgcccccccgccggcgagctgggcagcatcggcgaccacgagcgcgccatcgacttcagcccgtacctggagccgctgggcgcgccgcaggccccggcgcccgccacggccacggacaccttcgaggcggctccgcccgcgcccgcccccgcgcccgcctcctccgggcagcaccacgacttcctctccgacctcttctccgacgactacgggggcaagaactgcaagaagccggccgagtacggctacgtgagcctggggcgcctgggggccgccaagggcgcgctgcaccccggctgcttcgcgcccctgcacccaccgcccccgccgccgccgccgcccgccgagctcaaggcggagccgggcttcgagcccgcggactgcaagcggaaggaggaggccggggcgccgggcggcggcgcaggcatggcggcgggcttcccgtacgcgctgcgcgcttacctcggctaccaggcggtgccgagcggcagcagcgggagcctctccacgtcctcctcgtccagcccgcccggcacgccgagccccgctgacgccaaggcgcccccgaccgcctgctacgcgggggccgcgccggcgccctcgcaggtcaagagcaaggccaagaagaccgtggacaagcacagcgacgagtacaagatccggcgcgagcgcaacaacatcgccgtgcgcaagagccgcgacaaggccaagatgcgcaacctggagacgcagcacaaggtcctggagctcacggccgagaacgagcggctgcagaagaaggtggagcagctgtcgcgcgagctcagcaccctgcggaacttgttcaagcagctgcccgagcccctgctcgcctcctccggccactgctagcgcggcccccgcgcgcgtccccctgccggccggggctgagactccggggagcgcccgcgcccgcgccctcgcccccgcccccggcggcgccggcaaaactttggcactggggcacttggcagcgcggggagcccgtcggtaattttaatattttattatatatatatatctatatttttgtccaaaccaaccgcacatgcagatggggctcccgcccgtggtgttatttaaagaagaaacgtctatgtgtacagatgaatgataaactctctgcttctccctctgcccctctccaggcgccggcgggcgggccggtttcgaagttgatgcaatcggtttaaacatggctgaacgcgtgtgtacacgggactgacgcaacccacgtgtaactgtcagccgggccctgagtaatcgcttaaagatgttcctacgggcttgttgctgttgatgttttgttttgttttgttttttggtctttttttgtattataaaaaataatctatttctatgagaaaagaggcgtctgtatattttgggaatcttttccgtttcaagcattaagaacacttttaataaacttttttttgagaatggttacaaagcc

SEQ ID NO: 4 - protein, 345 aa, Homo sapiens
MQRLVAWDPACLPLPPPPPAFKSMEVANFYYEADCLAAAYGGKAAPAAPPAARPGPRPPAGELGSIGDHERAIDFSPYLEPLGAPQAPAPATATDTFEAAPPAPAPAPASSGQHHDFLSDLFSDDYGGKNCKKPAEYGYVSLGRLGAAKGALHPGCFAPLHPPPPPPPPPAELKAEPGFEPADCKRKEEAGAPGGGAGMAAGFPYALRAYLGYQAVPSGSSGSLSTSSSSSPPGTPSPADAKAPPTACYAGAAPAPSQVKSKAKKTVDKHSDEYKIRRERNNIAVRKSRDKAKMRNLETQHKVLELTAENERLQKKVEQLSRELSTLRNLFKQLPEPLLASSGHC

SEQ ID NO: 5 - DNA, 8738 bp, Homo sapiens
ctgacaatgctggggagatgaagatagtgtgtggctgcttctggactcaaggaggaggagagagattccgcgagccgacaccatgcgatccaaggcgagggcgaggaagctagccaaaagtgacggtgacgttgtaaataatatgtatgagcccaaccgggacctgctggccagccacagcgcggaggacgaggccgaggacagtgccatgtcgcccatccccgtggggccaccgtcccccttccccaccagcgaggacttcacccccaaggagggctcgccgtacgaggcccctgtctacattcctgaagacattccgatcccagcagacttcgagctccgagagtcctccatcccaggggctggcctgggggtctgggccaagaggaagatggaagccggggagaggctgggcccctgcgtggtggtgccccgggcggcggcaaaggagacagacttcggatgggagcaaatactgacggacgtggaagtgtcgccccaggaaggctgcatcacaaagatctccgaagacctgggcagtgagaagttctgcgtggatgcaaatcaggcgggggctggcagctggctcaagtacatccgtgtggcgtgctcctgcgatgaccagaacctcaccatgtgtcagatcagtgagcagatttactataaagtcattaaggacattgagccaggtgaggagctgctggtgcacgtgaaggaaggcgtctaccccctgggcacagtgccgcccggcctggacgaggagcccacgttccgctgtgacgagtgtgacgaactcttccagtccaagctggacctgcggcgccataagaagtacacgtgtggctcagtgggggctgcgctctacgagggcctggctgaggagctcaagcccgagggccttggcggtggcagcggccaagcccacgagtgcaaggactgcgagcggatgttccccaacaagtacagcctggagcagcacatggtcatccacacggaggagcgcgagtacaaatgcgaccagtgtcccaaggccttcaactggaagtccaacctcatccgccaccagatgtcccacgacagcggcaaacgcttcgaatgtgaaaactgcgtgaaggtgttcacggaccccagcaaccttcagcggcacatccgctcgcagcacgtgggcgctcgggcccacgcctgccccgactgcgggaagaccttcgccacgtcctccggcctcaagcagcacaagcatatccacagcacggtgaagcctttcatatgtgaggtctgccacaagtcctacacgcagttctccaacctgtgccggcacaagcggatgcacgccgactgccgcacgcagatcaagtgcaaggactgtggccagatgttcagcactacctcctccctcaacaagcaccggcgcttctgcgagggcaagaaccattacacgccgggcggcatctttgccccgggcctgcccttgacccccagccccatgatggacaaggcaaaaccctcccccagcctcaatcacgccagcctgggcttcaacgagtactttccctccaggccgcacccggggagcctgcccttctccacggcgcctcccacgttccccgcactcacccccggcttcccgggcatcttccctccatccttgtacccccggccgcctctgctacctcccacatcgctgctcaagagccccctgaaccacacccaggacgccaagctccccagtcccctggggaacccagccctgcccctggtctccgccgtcagcaacagcagccagggcacgacggcagctgcggggcccgaggagaagttcgagagccgcctggaggactcctgtgtggagaagctgaagaccaggagcagcgacatgtcggacggcagtgactttgaggacgtcaacaccaccacggggaccgacctggacacgaccacggggacgggctcggacctggacagcgacgtggacagcgaccctgacaaggacaagggcaagggcaagtccgccgagggccagcccaagtttgggggcggcttggcgcccccgggggccccgaacagcgtggccgaggtgcctgtcttctattcccagcactcattcttcccgccacccgacgagcagctgctgactgcaacgggcgccgccggggactccatcaaggccatcgcatccattgccgagaagtactttggccccggcttcatggggatgcaggagaagaagctgggctcgctcccctaccactcggcgttccccttccagttcctgcccaacttcccccactccctttaccccttcacggaccgagccctcgcccacaacttgctggtcaaggccgagccaaagtcaccccgggacgccctcaaggtgggcggccccagtgccgagtgcccctttgatctcaccaccaagcccaaagacgtgaagcccatcctgcccatgcccaagggcccctcggcccccgcatccggcgaggagcagccgctggacctgagcatcggcagccgggcccgtgccagccaaaacggcggcgggcgggagccccgcaagaaccacgtctatggggaacgcaagctgggcgccggcgaggggctgccccaggtgtgcccggcgcggatgccccagcagcccccgctccactacgccaagccctcgcccttcttcatggaccccatctacagcagggtagaaaagcggaaggtcacagaccccgtgggagccctgaaggagaagtacctgcggccgtccccgctgctcttccacccccagatgtcagccatagagaccatgacagagaagctggagagctttgcagccatgaaggcggactcgggcagctccctgcagcccctcccccaccaccccttcaacttccggtccccacccccaacgctctccgaccccatcctcaggaagggcaaggagcgatacacgtgcaggtactgtgggaagatcttccccagatcagccaatctcaccagacacctgaggacgcacactggggagcagccgtacaggtgtaagtactgcgaccgctccttcagcatctcttcgaacctccagcggcacgtccggaacatccacaacaaggagaagcctttcaagtgccacctgtgcaaccgctgcttcgggcagcagaccaacctggaccggcacctcaagaagcacgagcacgagaacgcaccagtgagccagcaccccggggtcctcacgaaccacctggggaccagcgcgtcctctcccacctcagagtcggacaaccacgcacttttagacgagaaagaagactcttatttctcggaaatcagaaactttattgccaatagtgagatgaaccaagcatcaacgcgaacagagaaacgggcggacatgcagatcgtggacggcagtgcccagtgtccaggcctagccagtgagaagcaggaggacgtggaggaggaggacgacgatgacctggaggaggacgatgaggacagcctggccgggaagtcgcaggatgacaccgtgtcccccgcacccgagccccaggccgcctacgaggatgaggaggatgaggagccagccgcctccctggccgtgggctttgaccacacccgaaggtgtgctgaggaccacgaaggcggtctgttagctttggagccgatgccgacttttgggaaggggctggacctccgcagagcagctgaggaagcatttgaagttaaagatgtgcttaattccaccttagattctgaggctttaaaacatacactgtgcaggcaggctaagaaccaggcatatgcaatgatgctgtccctttccgaagacactcctctccacaccccctcccagggttctctggacgcttggttgaaggtcactggagccacgtcggagtctggagcatttcaccccatcaaccacctctgacgggctgggcagccgggggccggtggccagagcgagggcaccagccacgaaggacggaggcgggcggggccccggagaaccctgtccctgcgtgtggccactcctcagcatcctccccacccaccatggttcattccgacttttccaatggaaactcagatcccaaaagtccctaaagcagtcgtagagtctcaccatctccaaggattggtcttgagaacactgttcagtgacggccatgcaggtggccgtccaaagacagccaacggagctgcctcgcagaatcagccagtgggcaggtggacgctctgctgagacagaagctggtggccactgccgggtgcccgcgtggggtcgcggaagggaatggatagactggtgtgctcaaaagagagagatcactcaaatgatttttataatgaaatgacaagaataacccttttggtaaccgtattgactgcagagtctatttaagcatgtggttttaaaaatagacagtattttttaaaaatcaaaaaatgacttgcaaattgttttttaaaagtaattttgcattgctttgaaatttgagctcatttgcaaacccgagtctgcctgggaacccgcactgtgcctgggtgtattctttatactgtagataatggagaaattttctatctctgtccctatttgtataagccaaggtgatgctgggtgccccgaggcagaacaagaggcgcggggccacacccgtgaaccatgcagacggccgaagaagtcttaggcagggcgccctgggctgcaggcctgcccgaggctgggatgggaagtgtgcctgccctcgtgtgacatggaattggtgtcaggaccgccacgtggccttcagaggaatccacaggtccccacccaagatccctcaattatatggggaagtcgagggcctgtggcttggatccgccatgcagagatgtggccgggcacccatcttccttccctcctctgtccctgcctcggccaccccacgcgggaacccagcgccgtcctctgaaggcagggccttggccacgtcctgggtctcccacctcccacctgaccccagcggctccggtgtcctccacgtggctgccctggggagcaatcccagcggatcgctccgggccaccaagccgcacctgtgcctgagactccggatggacgacacagtcgtcacgtcgctcttcctgcgggttcttggcgagacacagcttgagaacagaagggcgtcgggggaacctgccgcaaggagcagagacagcacagccccccgggcccagccgcctccctctcttgggacgcaacttcttccccactcggatgggctttaaattattcccataggggccaatttcaaataataatttttttccctgatggaatttaccttaatctgtatataacttgtaattttttctaattcatttcttttcttattttatttcctccttaacagtatttttggcattagacattcttattgtgaagaaataatgttaatataagtatctggtgaaggaccaaaaccgtgtgataaggttgtgtgtcgtgtgggagtggggcgattttttatgtgccaaatacccccgtcccccccatgaatcctgctgtccctgctgccgtttaccagacaatcatatgtttttgttaaatttgcgtttcagttacatttgcatttaagacaagtgttctatttatttcttgtattgtttggaagaaaaaatgatgatagagtcccaaaaagaagagaaaaaaaatgcccaagttgccctttaaaaaaaaagagcgtaaatacaaacaggagtggtgcaagccgccttggtgtgggtttgtgtcacgtgtggacatctcctcaggctttgtgtcacgcgtggacatctcctcaggctgtccccagcggtgacgggaggtgtcctggctgctccaggacaaaagacaatcgtctctgtgggtgccgggtggtccaggcttgcactgaagacgtgccacggggaggctcctgcaggaggctcaacccgacggatcacagtgaaagggattcctcccacgccagatctgcacaacgaggcaagacaggacccacctgtgcgtgcgctggggccatggggtggccccgccggggcagcgggggagctgcctgcagaagagccagctggcgtgtcgggaaggatccaggatctgcaaacacaactgctcaggccttctcacgcgtttccacaacatcccctgggtcagacccaccaggtaccccgtaggaatttccagtttcccttgatctagatgggattcttataaaaattcaacctcagacataaacaccccatttctgtaaacccaaattatatggtttcttctgcgaaagagtaaggtgtgtgcttttttttttttgcaatatgaccccgtctctctgaagtgggacattcggacggatggagccctcagcgtgtcttttcagcaggagcagaaccgatgagagccgcccttaccgttggtctccggatcccccagtcccatcccgccgttttcggctgtcttcctaaccgtcctgtcttctcttggcgctctttccttccacctttcccaagagtcctggttgcacgttttaagtcatatattttcgtccccctgaaaatgatggcaagcccagtttctcctgagcattcagacccccaggccccagcacttggcgttttcaggaggccctgttcttagagcccctgacaaaggcagcacttatttcctgggctggtgcgccccaaaacacggccccgacacttagtgtggccccaggccccagcgagcctcgccctcccagttttgctctgcccagcagtgttggtgcccagagatgacaagggccagggagcctggcccgggtgtgagaattcagagattctggcctccagctgtcaccacaccgtaacggggccatgtaactgtgcagcatggacagggatgcgacggggcagctggctgtgtccatggccaggtggccagggtcagggctgcaagccaggggtccagggcccttccgttcagcccaaatgctgccccaatgctaactccttggattgtcaacccccatcccccaaatggaaattccgaaggaggcctcctcgcacctgccctccgctgctcctcagaccccagcccccagcgagccgacgtccccacccgttcctgctctcatccccaggttgggcacgtggggttcctcctctgtgggcctggcagacccttcatgagtgggacccaagatatcactgacttcaacccagaggatcgagcccctgcaccctgcctggggccctggggtgtggagcagtggctggggtgggcgtggtgtggcctgagagactgcccagctggagaggccttcctttacaaggccacgcgtgcagctgtcccatccagaccccgactggccaagacctccacgtccccagagtccagccctggaaattccaagggccctggcgtcctctgccttccccgcttccccatgagcgtctgcaaaacacttgcctgaatacatatcacgtattttagactcgaagcctcaaagcactggattgtggtcccctgccccctctgtcccgtccccctgcccaagtgactgaaacctactgagctatattcactgtgctgtcctagggggagggagagcagagctcgcccctgcactgcagccttgtgggggagggcaaggctctcctcccagccagggacgccaggacatagctgctcctggtcagtggaggtcagccgggtatcaaaagccatgaaactgtgtctctgtagcaatgagtgatactgtgacaaaaccatccttgcattcttcctagaagagttcctctgctccttccattccatttttgtgtttgttttgttcttttctgtcactgatccgtattaccacttttggaaaaaaataaataaataaataaataaaaggcagcttgagtttccaaacgtgtgattcacttgtgaacaaaagtcattctaacaattgccttcagcgtcacgtgcattgccactgcgctttcggcacgagggatgctgagccctggtgtcagagtcgtaatttaaagcgtgtgtgtatatggactttgtcccttaaggtcgatataaagaatcctcgcagaatcacagacctgtgccgcccgccaccttctgccattgttacattacagatttggtttagttttgttttgttttgttttttcttttagaactgtatagtattgaaaaagaaatcaaatgtaaatgtctggttttcatataatgtttaaaaagaccattgagaaggaggctggcgctcgccccatgtcccccttgattgtaaattgcttctgttctgtttataagtaaactgtgcatgactcctgcttagcggtcattatcgtgtctgttggtgaaatttttattaaaaggaaaattctgtagatgcacttattgaatatgtgattaggatctacgtctgagactaggagtcctgaactgctgacgcgaaagaggcgcagttcccaattaatacggaaatcgctgtgggagaagaatgaaataagacgtgaagtgtaggaaatcatgaaaagaacaattttgcaaattgcattctgatgcttgtgatgaacacaaatgtacttgtgtagagacatttccttaagagaaagcctaggagaagccgatttggaggttaatgctgtagaataggactgtataccaaatgtaatctttccaatgctccaatgaatttatacatgagattgatatgcaataaatctgtgtgcttttctaaaaaaaaaaaaaaaaa

SEQ ID NO: 6 - DNA, 8674 bp, Homo sapiens
ctgacaatgctggggagatgaagatagtgtgtggctgcttctggactcaaggaggaggagagagattccgcgagccgacaccatgcgatccaaggcgagggcgaggaagctagccaaaagtgacggtgacgttgtaaataatatgtatgagcccaaccgggacctgctggccagccacagcgcggaggacgaggccgaggacagtgccatgtcgcccatccccgtggggccaccgtcccccttccccaccagcgaggacttcacccccaaggagggctcgccgtacgaggcccctgtctacattcctgaagacattccgatcccagcagacttcgagctccgagagtcctccatcccaggggctggcctgggggtctgggccaagaggaagatggaagccggggagaggctgggcccctgcgtggtggtgccccgggcggcggcaaaggagacagacttcggatgggagcaaatactgacggacgtggaagtgtcgccccaggaaggctgcatcacaaagatctccgaagacctgggcagtgagaagttctgcgtggatgcaaatcaggcgggggctggcagctggctcaagtacatccgtgtggcgtgctcctgcgatgaccagaacctcaccatgtgtcagatcagtgagcagatttactataaagtcattaaggacattgagccaggtgaggagctgctggtgcacgtgaaggaaggcgtctaccccctgggcacagtgccgcccggcctggacgaggagcccacgttccgctgtgacgagtgtgacgaactcttccagtccaagctggacctgcggcgccataagaagtacacgtgtggctcagtgggggctgcgctctacgagggcctggctgaggagctcaagcccgagggccttggcggtggcagcggccaagcccacgagtgcaaggactgcgagcggatgttccccaacaagtacagcctggagcagcacatggtcatccacacggaggagcgcgagtacaaatgcgaccagtgtcccaaggccttcaactggaagtccaacctcatccgccaccagatgtcccacgacagcggcaaacgcttcgaatgtgaaaactgcgtgaaggtgttcacggaccccagcaaccttcagcggcacatccgctcgcagcacgtgggcgctcgggcccacgcctgccccgactgcgggaagaccttcgccacgtcctccggcctcaagcagcacaagcatatccacagcacggtgaagcctttcatatgtgaggtctgccacaagtcctacacgcagttctccaacctgtgccggcacaagcggatgcacgccgactgccgcacgcagatcaagtgcaaggactgtggccagatgttcagcactacctcctccctcaacaagcaccggcgcttctgcgagggcaagaaccattacacgccgggcggcatctttgccccgggcctgcccttgacccccagccccatgatggacaaggcaaaaccctcccccagcctcaatcacgccagcctgggcttcaacgagtactttccctccaggccgcacccggggagcctgcccttctccacggcgcctcccacgttccccgcactcacccccggcttcccgggcatcttccctccatccttgtacccccggccgcctctgctacctcccacatcgctgctcaagagccccctgaaccacacccaggacgccaagctccccagtcccctggggaacccagccctgcccctggtctccgccgtcagcaacagcagccagggcacgacggcagctgcggggcccgaggagaagttcgagagccgcctggaggactcctgtgtggagaagctgaagaccaggagcagcgacatgtcggacggcagtgactttgaggacgtcaacaccaccacggggaccgacctggacacgaccacggggacgggctcggacctggacagcgacgtggacagcgaccctgacaaggacaagggcaagggcaagtccgccgagggccagcccaagtttgggggcggcttggcgcccccgggggccccgaacagcgtggccgaggtgcctgtcttctattcccagcactcattcttcccgccacccgacgagcagctgctgactgcaacgggcgccgccggggactccatcaaggccatcgcatccattgccgagaagtactttggccccggcttcatggggatgcaggagaagaagctgggctcgctcccctaccactcggcgttccccttccagttcctgcccaacttcccccactccctttaccccttcacggaccgagccctcgcccacaacttgctggtcaaggccgagccaaagtcaccccgggacgccctcaaggtgggcggccccagtgccgagtgcccctttgatctcaccaccaagcccaaagacgtgaagcccatcctgcccatgcccaagggcccctcggcccccgcatccggcgaggagcagccgctggacctgagcatcggcagccgggcccgtgccagccaaaacggcggcgggcgggagccccgcaagaaccacgtctatggggaacgcaagctgggcgccggcgaggggctgccccaggtgtgcccggcgcggatgccccagcagcccccgctccactacgccaagccctcgcccttcttcatggaccccatctacagcagggtagaaaagcggaaggtcacagaccccgtgggagccctgaaggagaagtacctgcggccgtccccgctgctcttccacccccagatgtcagccatagagaccatgacagagaagctggagagctttgcagccatgaaggcggactcgggcagctccctgcagcccctcccccaccaccccttcaacttccggtccccacccccaacgctctccgaccccatcctcaggaagggcaaggagcgatacacgtgcaggtactgtgggaagatcttccccagatcagccaatctcaccagacacctgaggacgcacactggggagcagccgtacaggtgtaagtactgcgaccgctccttcagcatctcttcgaacctccagcggcacgtccggaacatccacaacaaggagaagcctttcaagtgccacctgtgcaaccgctgcttcgggcagcagaccaacctggaccggcacctcaagaagcacgagcacgagaacgcaccagtgagccagcaccccggggtcctcacgaaccacctggggaccagcgcgtcctctcccacctcagagtcggacaaccacgcacttttagacgagaaagaagactcttatttctcggaaatcagaaactttattgccaatagtgagatgaaccaagcatcaacgcgaacagagaaacgggcggacatgcagatcgtggacggcagtgcccagtgtccaggcctagccagtgagaagcaggaggacgtggaggaggaggacgacgatgacctggaggaggacgatgaggacagcctggccgggaagtcgcaggatgacaccgtgtcccccgcacccgagccccaggccgcctacgaggatgaggaggatgaggagccagccgcctccctggccgtgggctttgaccacacccgaaggtgtgctgaggaccacgaaggcggtctgttagctttggagccgatgccgacttttgggaaggggctggacctccgcagagcagctgaggaagcatttgaagttaaagatgtgcttaattccaccttagattctgaggctttaaaacatacactgtgcaggcaggctaagaaccagggttctctggacgcttggttgaaggtcactggagccacgtcggagtctggagcatttcaccccatcaaccacctctgacgggctgggcagccgggggccggtggccagagcgagggcaccagccacgaaggacggaggcgggcggggccccggagaaccctgtccctgcgtgtggccactcctcagcatcctccccacccaccatggttcattccgacttttccaatggaaactcagatcccaaaagtccctaaagcagtcgtagagtctcaccatctccaaggattggtcttgagaacactgttcagtgacggccatgcaggtggccgtccaaagacagccaacggagctgcctcgcagaatcagccagtgggcaggtggacgctctgctgagacagaagctggtggccactgccgggtgcccgcgtggggtcgcggaagggaatggatagactggtgtgctcaaaagagagagatcactcaaatgatttttataatgaaatgacaagaataacccttttggtaaccgtattgactgcagagtctatttaagcatgtggttttaaaaatagacagtattttttaaaaatcaaaaaatgacttgcaaattgttttttaaaagtaattttgcattgctttgaaatttgagctcatttgcaaacccgagtctgcctgggaacccgcactgtgcctgggtgtattctttatactgtagataatggagaaattttctatctctgtccctatttgtataagccaaggtgatgctgggtgccccgaggcagaacaagaggcgcggggccacacccgtgaaccatgcagacggccgaagaagtcttaggcagggcgccctgggctgcaggcctgcccgaggctgggatgggaagtgtgcctgccctcgtgtgacatggaattggtgtcaggaccgccacgtggccttcagaggaatccacaggtccccacccaagatccctcaattatatggggaagtcgagggcctgtggcttggatccgccatgcagagatgtggccgggcacccatcttccttccctcctctgtccctgcctcggccaccccacgcgggaacccagcgccgtcctctgaaggcagggccttggccacgtcctgggtctcccacctcccacctgaccccagcggctccggtgtcctccacgtggctgccctggggagcaatcccagcggatcgctccgggccaccaagccgcacctgtgcctgagactccggatggacgacacagtcgtcacgtcgctcttcctgcgggttcttggcgagacacagcttgagaacagaagggcgtcgggggaacctgccgcaaggagcagagacagcacagccccccgggcccagccgcctccctctcttgggacgcaacttcttccccactcggatgggctttaaattattcccataggggccaatttcaaataataatttttttccctgatggaatttaccttaatctgtatataacttgtaattttttctaattcatttcttttcttattttatttcctccttaacagtatttttggcattagacattcttattgtgaagaaataatgttaatataagtatctggtgaaggaccaaaaccgtgtgataaggttgtgtgtcgtgtgggagtggggcgattttttatgtgccaaatacccccgtcccccccatgaatcctgctgtccctgctgccgtttaccagacaatcatatgtttttgttaaatttgcgtttcagttacatttgcatttaagacaagtgttctatttatttcttgtattgtttggaagaaaaaatgatgatagagtcccaaaaagaagagaaaaaaaatgcccaagttgccctttaaaaaaaaagagcgtaaatacaaacaggagtggtgcaagccgccttggtgtgggtttgtgtcacgtgtggacatctcctcaggctttgtgtcacgcgtggacatctcctcaggctgtccccagcggtgacgggaggtgtcctggctgctccaggacaaaagacaatcgtctctgtgggtgccgggtggtccaggcttgcactgaagacgtgccacggggaggctcctgcaggaggctcaacccgacggatcacagtgaaagggattcctcccacgccagatctgcacaacgaggcaagacaggacccacctgtgcgtgcgctggggccatggggtggccccgccggggcagcgggggagctgcctgcagaagagccagctggcgtgtcgggaaggatccaggatctgcaaacacaactgctcaggccttctcacgcgtttccacaacatcccctgggtcagacccaccaggtaccccgtaggaatttccagtttcccttgatctagatgggattcttataaaaattcaacctcagacataaacaccccatttctgtaaacccaaattatatggtttcttctgcgaaagagtaaggtgtgtgcttttttttttttgcaatatgaccccgtctctctgaagtgggacattcggacggatggagccctcagcgtgtcttttcagcaggagcagaaccgatgagagccgcccttaccgttggtctccggatcccccagtcccatcccgccgttttcggctgtcttcctaaccgtcctgtcttctcttggcgctctttccttccacctttcccaagagtcctggttgcacgttttaagtcatatattttcgtccccctgaaaatgatggcaagcccagtttctcctgagcattcagacccccaggccccagcacttggcgttttcaggaggccctgttcttagagcccctgacaaaggcagcacttatttcctgggctggtgcgccccaaaacacggccccgacacttagtgtggccccaggccccagcgagcctcgccctcccagttttgctctgcccagcagtgttggtgcccagagatgacaagggccagggagcctggcccgggtgtgagaattcagagattctggcctccagctgtcaccacaccgtaacggggccatgtaactgtgcagcatggacagggatgcgacggggcagctggctgtgtccatggccaggtggccagggtcagggctgcaagccaggggtccagggcccttccgttcagcccaaatgctgccccaatgctaactccttggattgtcaacccccatcccccaaatggaaattccgaaggaggcctcctcgcacctgccctccgctgctcctcagaccccagcccccagcgagccgacgtccccacccgttcctgctctcatccccaggttgggcacgtggggttcctcctctgtgggcctggcagacccttcatgagtgggacccaagatatcactgacttcaacccagaggatcgagcccctgcaccctgcctggggccctggggtgtggagcagtggctggggtgggcgtggtgtggcctgagagactgcccagctggagaggccttcctttacaaggccacgcgtgcagctgtcccatccagaccccgactggccaagacctccacgtccccagagtccagccctggaaattccaagggccctggcgtcctctgccttccccgcttccccatgagcgtctgcaaaacacttgcctgaatacatatcacgtattttagactcgaagcctcaaagcactggattgtggtcccctgccccctctgtcccgtccccctgcccaagtgactgaaacctactgagctatattcactgtgctgtcctagggggagggagagcagagctcgcccctgcactgcagccttgtgggggagggcaaggctctcctcccagccagggacgccaggacatagctgctcctggtcagtggaggtcagccgggtatcaaaagccatgaaactgtgtctctgtagcaatgagtgatactgtgacaaaaccatccttgcattcttcctagaagagttcctctgctccttccattccatttttgtgtttgttttgttcttttctgtcactgatccgtattaccacttttggaaaaaaataaataaataaataaataaaaggcagcttgagtttccaaacgtgtgattcacttgtgaacaaaagtcattctaacaattgccttcagcgtcacgtgcattgccactgcgctttcggcacgagggatgctgagccctggtgtcagagtcgtaatttaaagcgtgtgtgtatatggactttgtcccttaaggtcgatataaagaatcctcgcagaatcacagacctgtgccgcccgccaccttctgccattgttacattacagatttggtttagttttgttttgttttgttttttcttttagaactgtatagtattgaaaaagaaatcaaatgtaaatgtctggttttcatataatgtttaaaaagaccattgagaaggaggctggcgctcgccccatgtcccccttgattgtaaattgcttctgttctgtttataagtaaactgtgcatgactcctgcttagcggtcattatcgtgtctgttggtgaaatttttattaaaaggaaaattctgtagatgcacttattgaatatgtgattaggatctacgtctgagactaggagtcctgaactgctgacgcgaaagaggcgcagttcccaattaatacggaaatcgctgtgggagaagaatgaaataagacgtgaagtgtaggaaatcatgaaaagaacaattttgcaaattgcattctgatgcttgtgatgaacacaaatgtacttgtgtagagacatttccttaagagaaagcctaggagaagccgatttggaggttaatgctgtagaataggactgtataccaaatgtaatctttccaatgctccaatgaatttatacatgagattgatatgcaataaatctgtgtgcttttctaaaaaaaaaa

SEQ ID NO: 7 - protein, 1276 aa, Homo sapiens
MRSKARARKLAKSDGDVVNNMYEPNRDLLASHSAEDEAEDSAMSPIPVGPPSPFPTSEDFTPKEGSPYEAPVYIPEDIPIPADFELRESSIPGAGLGVWAKRKMEAGERLGPCVVVPRAAAKETDFGWEQILTDVEVSPQEGCITKISEDLGSEKFCVDANQAGAGSWLKYIRVACSCDDQNLTMCQISEQIYYKVIKDIEPGEELLVHVKEGVYPLGTVPPGLDEEPTFRCDECDELFQSKLDLRRHKKYTCGSVGAALYEGLAEELKPEGLGGGSGQAHECKDCERMFPNKYSLEQHMVIHTEEREYKCDQCPKAFNWKSNLIRHQMSHDSGKRFECENCVKVFTDPSNLQRHIRSQHVGARAHACPDCGKTFATSSGLKQHKHIHSTVKPFICEVCHKSYTQFSNLCRHKRMHADCRTQIKCKDCGQMFSTTSSLNKHRRFCEGKNHYTPGGIFAPGLPLTPSPMMDKAKPSPSLNHASLGFNEYFPSRPHPGSLPFSTAPPTFPALTPGFPGIFPPSLYPRPPLLPPTSLLKSPLNHTQDAKLPSPLGNPALPLVSAVSNSSQGTTAAAGPEEKFESRLEDSCVEKLKTRSSDMSDGSDFEDVNTTTGTDLDTTTGTGSDLDSDVDSDPDKDKGKGKSAEGQPKFGGGLAPPGAPNSVAEVPVFYSQHSFFPPPDEQLLTATGAAGDSIKAIASIAEKYFGPGFMGMQEKKLGSLPYHSAFPFQFLPNFPHSLYPFTDRALAHNLLVKAEPKSPRDALKVGGPSAECPFDLTTKPKDVKPILPMPKGPSAPASGEEQPLDLSIGSRARASQNGGGREPRKNHVYGERKLGAGEGLPQVCPARMPQQPPLHYAKPSPFFMDPIYSRVEKRKVTDPVGALKEKYLRPSPLLFHPQMSAIETMTEKLESFAAMKADSGSSLQPLPHHPFNFRSPPPTLSDPILRKGKERYTCRYCGKIFPRSANLTRHLRTHTGEQPYRCKYCDRSFSISSNLQRHVRNIHNKEKPFKCHLCNRCFGQQTNLDRHLKKHEHENAPVSQHPGVLTNHLGTSASSPTSESDNHALLDEKEDSYFSEIRNFIANSEMNQASTRTEKRADMQIVDGSAQCPGLASEKQEDVEEEDDDDLEEDDEDSLAGKSQDDTVSPAPEPQAAYEDEEDEEPAASLAVGFDHTRRCAEDHEGGLLALEPMPTFGKGLDLRRAAEEAFEVKDVLNSTLDSEALKHTLCRQAKNQAYAMMLSLSEDTPLHTPSQGSLDAWLKVTGATSESGAFHPINHL

SEQ ID NO: 8 - protein, 1257 aa, Homo sapiens
MRSKARARKLAKSDGDVVNNMYEPNRDLLASHSAEDEAEDSAMSPIPVGPPSPFPTSEDFTPKEGSPYEAPVYIPEDIPIPADFELRESSIPGAGLGVWAKRKMEAGERLGPCVVVPRAAAKETDFGWEQILTDVEVSPQEGCITKISEDLGSEKFCVDANQAGAGSWLKYIRVACSCDDQNLTMCQISEQIYYKVIKDIEPGEELLVHVKEGVYPLGTVPPGLDEEPTFRCDECDELFQSKLDLRRHKKYTCGSVGAALYEGLAEELKPEGLGGGSGQAHECKDCERMFPNKYSLEQHMVIHTEEREYKCDQCPKAFNWKSNLIRHQMSHDSGKRFECENCVKVFTDPSNLQRHIRSQHVGARAHACPDCGKTFATSSGLKQHKHIHSTVKPFICEVCHKSYTQFSNLCRHKRMHADCRTQIKCKDCGQMFSTTSSLNKHRRFCEGKNHYTPGGIFAPGLPLTPSPMMDKAKPSPSLNHASLGFNEYFPSRPHPGSLPFSTAPPTFPALTPGFPGIFPPSLYPRPPLLPPTSLLKSPLNHTQDAKLPSPLGNPALPLVSAVSNSSQGTTAAAGPEEKFESRLEDSCVEKLKTRSSDMSDGSDFEDVNTTTGTDLDTTTGTGSDLDSDVDSDPDKDKGKGKSAEGQPKFGGGLAPPGAPNSVAEVPVFYSQHSFFPPPDEQLLTATGAAGDSIKAIASIAEKYFGPGFMGMQEKKLGSLPYHSAFPFQFLPNFPHSLYPFTDRALAHNLLVKAEPKSPRDALKVGGPSAECPFDLTTKPKDVKPILPMPKGPSAPASGEEQPLDLSIGSRARASQNGGGREPRKNHVYGERKLGAGEGLPQVCPARMPQQPPLHYAKPSPFFMDPIYSRVEKRKVTDPVGALKEKYLRPSPLLFHPQMSAIETMTEKLESFAAMKADSGSSLQPLPHHPFNFRSPPPTLSDPILRKGKERYTCRYCGKIFPRSANLTRHLRTHTGEQPYRCKYCDRSFSISSNLQRHVRNIHNKEKPFKCHLCNRCFGQQTNLDRHLKKHEHENAPVSQHPGVLTNHLGTSASSPTSESDNHALLDEKEDSYFSEIRNFIANSEMNQASTRTEKRADMQIVDGSAQCPGLASEKQEDVEEEDDDDLEEDDEDSLAGKSQDDTVSPAPEPQAAYEDEEDEEPAASLAVGFDHTRRCAEDHEGGLLALEPMPTFGKGLDLRRAAEEAFEVKDVLNSTLDSEALKHTLCRQAKNQGSLDAWLKVTGATSESGAFHPINHL

SEQ ID NO: 9 - protein, 28 aa, Homo sapiens
MGETLGDSPIDPESDSFTDTLSANISQE

SEQ ID NO: 10 - protein, 505 aa, Mus musculus
MGETLGDSPVDPEHGAFADALPMSTSQEITMVDTEMPFWPTNFGISSVDLSVMEDHSHSFDIKPFTTVDFSSISAPHYEDIPFTRADPMVADYKYDLKLQEYQSAIKVEPASPPYYSEKTQLYNRPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKNIPGFINLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKVLQKMTDLRQIVTEHVQLLHVIKKTETDMSLHPLLQEIYKDLY

SEQ ID NO: 11 - protein, 505 aa, Rattus norvegicus [sequence omitted]

SEQ ID NO: 12 - protein, 296 aa, Mus musculus
MHRLLAWDAACLPPPPAAFRPMEVANFYYEPDCLAYGAKAARAAPRAPAAEPAIGEHERAIDFSPYLEPLAPAADFAAPAPAHHDFLSDLFADDYGAKPSKKPADYGYVSLGRAGAKAAPPACFPPPPPAALKAEPGFEPADCKRADDAPAMAAGFPFALRAYLGYQATPSGSSGSLSTSSSSSPPGTPSPADAKAAPAACFAGPPAAPAKAKAKKTVDKLSDEYKMRRERNNIAVRKSRDKAKMRNLETQHKVLELTAENERLQKKVEQLSRELSTLRNLFKQLPEPLLASAGHC

SEQ ID NO: 13 - protein, 297 aa, Rattus norvegicus
MHRLLAWDAACLPPPPAAFRPMEVANFYYEPDCLAYGAKAARAAPRAPAAEPAIGEHERAIDFSPYLEPLAPAAADFAAPAPAHHDFLSDLFADDYGAKPSKKPSDYGYVSLGRAGAKAAPPACFPPPPPAALKAEPGFEPADCKRADDAPAMAAGFPFALRAYLGYQATPSGSSGSLSTSSSSSPPGTPSPADAKAAPAACFAGPPAAPAKAKAKKAVDKLSDEYKMRRERNNIAVRKSRDKAKMRNLETQHKVLELTAENERLQKKVEQLSRELSTLRNLFKQLPEPLLASAGHC

SEQ ID NO: 14 - DNA, 13 bp, Artificial Sequence
  source: /note=“Description of Artificial Sequence Synthetic oligonucleotide”
aggtcanaggtca

SEQ ID NO: 15 - DNA, 20 bp, Artificial Sequence
  source: /note=“Description of Artificial Sequence Synthetic oligonucleotide”
caaaactaggtcaaaggtca

SEQ ID NO: 16 - DNA, 13 bp, Artificial Sequence
  source: /note=“Description of Artificial Sequence Synthetic oligonucleotide”
aggncaaaggtca

SEQ ID NO: 17 - DNA, 16 bp, Artificial Sequence
  source: /note=“Description of Artificial Sequence Synthetic oligonucleotide”
antgggncaaaggtca

SEQ ID NO: 18 - DNA, 9 bp, Artificial Sequence
  source: /note=“Description of Artificial Sequence Synthetic oligonucleotide”
tknngnaak

SEQ ID NO: 19 - DNA, 23 bp, Artificial Sequence
  source: /note=“Description of Artificial Sequence Synthetic oligonucleotide”
tcgcctagcatttcatcacacgt

SEQ ID NO: 20 - DNA, 20 bp, Artificial Sequence
  source: /note=“Description of Artificial Sequence Synthetic primer”
tgcaggtgatcaagaagacg

SEQ ID NO: 21 - DNA, 22 bp, Artificial Sequence
  source: /note=“Description of Artificial Sequence Synthetic primer”
gcaggagatctacaaggacttg

SEQ ID NO: 22 - DNA, 20 bp, Artificial Sequence
  source: /note=“Description of Artificial Sequence Synthetic primer”
ccacgcctgtccttagaaag

SEQ ID NO: 23 - DNA, 22 bp, Artificial Sequence
  source: /note=“Description of Artificial Sequence Synthetic primer”
gatgaagtcctgtcttggaagg

SEQ ID NO: 24 - DNA, 20 bp, Artificial Sequence
  source: /note=“Description of Artificial Sequence Synthetic primer”
tcatgaaaggcgtcacttcc

SEQ ID NO: 25 - DNA, 20 bp, Artificial Sequence
  source: /note=“Description of Artificial Sequence Synthetic primer”
ctcagtgtgctctccaagtg

SEQ ID NO: 26 - DNA, 24 bp, Artificial Sequence
  source: /note=“Description of Artificial Sequence Synthetic primer”
ctaagtaaaagggtggagaggttc

SEQ ID NO: 27 - DNA, 21 bp, Artificial Sequence
  source: /note=“Description of Artificial Sequence Synthetic primer”
tgacactggcaaaacaatgca

SEQ ID NO: 28 - DNA, 19 bp, Artificial Sequence
  source: /note=“Description of Artificial Sequence Synthetic primer”
cttcgcggggtagtgttgg

SEQ ID NO: 29 - DNA, 20 bp, Artificial Sequence
  source: /note=“Description of Artificial Sequence Synthetic primer”
atgtagttctgccccacagc

SEQ ID NO: 30 - DNA, 18 bp, Artificial Sequence
  source: /note=“Description of Artificial Sequence Synthetic primer”
tctacgacacggtccagg

SEQ ID NO: 31 - DNA, 25 bp, Artificial Sequence
  source: /note=“Description of Artificial Sequence Synthetic primer”
gccaaaccaacaactttatctcttc

SEQ ID NO: 32 - DNA, 21 bp, Artificial Sequence
  source: /note=“Description of Artificial Sequence Synthetic primer”
cccggttccacattgtaagag

SEQ ID NO: 33 - DNA, 21 bp, Artificial Sequence
  source: /note=“Description of Artificial Sequence Synthetic primer”
aacgcggagaagtggaacaag

SEQ ID NO: 34 - DNA, 21 bp, Artificial Sequence
  source: /note=“Description of Artificial Sequence Synthetic primer”
tttgtgggcctgaagaaaact

SEQ ID NO: 35 - DNA, 20 bp, Artificial Sequence
  source: /note=“Description of Artificial Sequence Synthetic primer”
gagcatttcaccccatcaac

SEQ ID NO: 36 - DNA, 20 bp, Artificial Sequence
  source: /note=“Description of Artificial Sequence Synthetic primer”
cacgcagaacttctcactgc

SEQ ID NO: 37 - DNA, 24 bp, Artificial Sequence
  source: /note=“Description of Artificial Sequence Synthetic primer”
caaattttgtaatccagaggttga

SEQ ID NO: 38 - DNA, 21 bp, Artificial Sequence
  source: /note=“Description of Artificial Sequence Synthetic primer”
ccctcagaatagtgcaactgg

SEQ ID NO: 39 - DNA, 21 bp, Artificial Sequence
  source: /note=“Description of Artificial Sequence Synthetic primer”
ccctccaccttcatgtagaac

SEQ ID NO: 40 - DNA, 22 bp, Artificial Sequence
  source: /note=“Description of Artificial Sequence Synthetic primer”
cagcacttagagatggagttgg

SEQ ID NO: 41 - DNA, 22 bp, Artificial Sequence
  source: /note=“Description of Artificial Sequence Synthetic primer”
gcttgctaaatcagggaaaaca

SEQ ID NO: 42 - DNA, 18 bp, Artificial Sequence
  source: /note=“Description of Artificial Sequence Synthetic primer”
cacccaggcggaagtctc

SEQ ID NO: 43 - DNA, 21 bp, Artificial Sequence
  source: /note=“Description of Artificial Sequence Synthetic primer”
ggtactccatctggctcattg

SEQ ID NO: 44 - DNA, 21 bp, Artificial Sequence
  source: /note=“Description of Artificial Sequence Synthetic primer”
ggtccttttcaccagcaagct

SEQ ID NO: 45 - DNA, 19 bp, Artificial Sequence
  source: /note=“Description of Artificial Sequence Synthetic primer”
ggccagacttcgacgacaa

SEQ ID NO: 46 - DNA, 20 bp, Artificial Sequence
  source: /note=“Description of Artificial Sequence Synthetic primer”
aaggacatgaggcccttttt

SEQ ID NO: 47 - DNA, 21 bp, Artificial Sequence
  source: /note=“Description of Artificial Sequence Synthetic primer”
gtctgactttcacgacctctg

SEQ ID NO: 48 - DNA, 25 bp, Artificial Sequence
  source: /note=“Description of Artificial Sequence Synthetic primer”
cacacttaaggtgcgttcaatagtc

SEQ ID NO: 49 - DNA, 22 bp, Artificial Sequence
  source: /note=“Description of Artificial Sequence Synthetic primer”
gtatgcagtcacagcgatgaat

SEQ ID NO: 50 - DNA, 19 bp, Artificial Sequence
  source: /note=“Description of Artificial Sequence Synthetic primer”
ctgtccgagtccaaatcgc

SEQ ID NO: 51 - DNA, 21 bp, Artificial Sequence
  source: /note=“Description of Artificial Sequence Synthetic primer”
agggctgtcctgaataagcag

SEQ ID NO: 52 - DNA, 20 bp, Artificial Sequence
  source: /note=“Description of Artificial Sequence Synthetic primer”
ggagtctggagcatttcacc

SEQ ID NO: 53 - DNA, 20 bp, Artificial Sequence
  source: /note=“Description of Artificial Sequence Synthetic primer”
atgggagcaaatactgacgg

(19) United States
(12) Patent Application Publication
(10) Pub. No.: US 2015/0004144 A1
(43) Pub. Date: Jan. 1, 2015

(54) DIFFERENTIATION INTO BROWN ADIPOCYTES

(71) Applicant: THE GENERAL HOSPITAL CORPORATION, Boston, MA (US)

(72) Inventors: Chad Cowan, Boston, MA (US); Robert Schinzel, Cambridge, MA (US); Tim Ahfeldt, Cambridge, MA (US); YounKyoung Lee, Cambridge, MA (US)

(73) Assignee: THE GENERAL HOSPITAL CORPORATION, Boston, MA (US)

(21) Appl. No.: 14/360,097

(22) PCT Filed: Nov. 28, 2012
(86) PCT No.: PCT/US2012/066785

§ 371 Date: May 22, 2014

(30) Foreign Application Priority Data
Dec. 2, 2011 (US) 61566278

(51) Int. Cl. G01N 33/50

(52) U.S. Cl. CPC G01N 33/5044; G01N 2500/04; G01N 2500/10
USPC 424/93.7; 435/377; 435/366; 435/34; 506/9; 435/7.21

(57) ABSTRACT

The technology described herein is directed to methods and compositions relating to the differentiation and activity of brown adipocytes, and the therapeutic uses thereof.

### CROSS-REFERENCE TO RELATED APPLICATIONS

This application claims benefit under 35 U.S.C. § 119(e) of U.S. Provisional Application No. 61/566,278 filed Dec. 2, 2011, the contents of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Nov. 19, 2012, is named 03025807,txt and is 81,990 bytes in size.

### TECHNICAL FIELD

The technology described herein is directed to methods and compositions relating to the differentiation and activity of brown adipocytes.

### BACKGROUND

Obesity is the second leading cause of preventable death in the U.S. It is a disease in which the natural energy reserve, stored in the adipose tissue of humans and other mammals, is increased to a point where it is associated with adverse health effects and mortality. The increased triglycerides, decreased HDL levels and abnormal LDL composition found in obese individuals are strongly indicated in the development of atherosclerosis and cardiovascular disease. Furthermore, obesity is associated with type 2 diabetes, metabolic disorders, and premature mortality. Despite recognition of the risks associated with obesity, methods for preventing or treating obesity and associated metabolic disorders are inadequate. Obesity continues to pose a significant public health problem.

Obesity is a complex, multi-factorial disease involving environmental, genetic, and behavioral components leading to an overall imbalance in energy intake to expenditure. Current estimates suggest that as many as 60 million Americans are obese (1 in every 3), and 9 million are severely obese. Alarmingly, the prevalence of obesity has almost tripled in adults and children over the past 50 years. Each year, obesity causes at least 300,000 deaths in the U.S., and healthcare costs associated with obesity are approximately $100 billion per year. Statistics such as these have caused many to view obesity as a national pandemic. To restore the balance, either a decrease in energy intake or an increase in energy expenditure is necessary. While dietary restriction andor physical activity have shown to be beneficial, a large proportion of obese individuals, given their environmental, genetic and behavioral background find it difficult to impossible to make those adjustments.

The known function of brown adipose tissue is thermoregulation. It achieves this goal by uncoupling the respiratory chain of the mitochondria membrane, thus dispersing heat instead of creating ATP. While this function and the general characteristics are well established in rodents, in human beings brown adipose tissue was long thought to be limited to infancy, with negligible influence on the energy balance. Recent studies show that this view needs to be revised and that brown adipose tissue can be found in large parts of the adult population in significant amounts, and intriguingly was found to be negatively correlated to the body mass index (BMI) of a person. Furthermore, gene targeting studies in mice indicate that a knock-out of a whole set of genes important in white fat metabolism can lead to a phenotype in mice characterized by an increased amount and increased activity of brown adipose tissue, which seem to compensate for the loss in white fat tissue. Intriguingly, these mice on a high fat diet (HFD) were resistant to obesity, while physical activity and food intake remain constant. Furthermore, while the control group on HFD became resistant to insulin, a first indicator of type 2 diabetes, the genetically altered mice remained responsive to insulin stimuli.

Both lines of reasoning, the negative correlation to BMI in humans and the resistance to obesity in mice, indicate that an increased energy expenditure in brown adipose tissue might be a possible mechanism to counteract the overall imbalance in energy intake to expenditure often leading to obesity. This has made brown adipose tissue an interesting target for potential drugs development, for possible transplantation therapy and for general basic research. Yet human brown adipose tissue is notoriously difficult to obtain.

A number of groups have developed human cell-based models for the study of adipogenesis using either mesenchymal stem cells (MSCs) from bone marrow or other tissues, ^{20, 21} or adipose-derived stromal vascular cells (ADSVCs) ^{22,23}. Although these cellular systems have proven useful ²⁴, they have limitations including limited proliferative potential, decreased differentiation with continued passaging ²⁵ and variable differentiation potential. To overcome these obstacles, several groups have sought to use human pluripotent stem cells (hPSCs) to generate human adipocytes; reports have been limited to white adipocytes ^{26,27,28,29}. Moreover, the efficient generation of large numbers of hPSC-derived adipocytes, with detailed phenotypic characterization that documents fidelity to primary cells, has remained elusive. In order to provide brown adipocytes for use in vitro or for implantation into subjects in need of brown adipocytes, it is necessary to develop reliable and scalable protocols for the differentiation of stem and progenitor cells into brown adipocytes.

### SUMMARY

Described herein are methods of promoting the differentiation of non-neuronal cells to brown adipocytes, based upon the inventors' discovery that differentiation to brown adipocytes can be caused by increased levels and/or activities of C/EBPβ and PPARγ2, without a requirement for increasing the level or activity of PRDM16. In some embodiments, the non-neuronal cells are stem or precursor cells.

In one aspect, the invention is directed to methods for promoting the differentiation of cells into brown adipocytes comprising (a) contacting a population of cells with at least one agent that increases the level of activity of PPARγ2 and C/EBPβ and (b) culturing the cells under conditions favorable for differentiation into brown adipocytes. The method does not comprise contacting the cells with an agent which increases the level of PRDM16. In one embodiment, the method can include providing a population of cells, which will be treated to promote the differentiation of cells into brown adipocytes.

The agent that increases the level or activity of PPARγ2 and C/EBPβ can comprise (a) a polynucleotide comprising a gene sequence that encodes a PPARγ2 and/or a C/EBPβ polypeptide, (b) a PPARγ2 polypeptide and/or C/EBPβ polypeptide or (c) a small molecule that increases the level or activity of PPARγ2 or C/EBPβ. A small molecule that increases the level or activity of PPARγ2 or C/EBPβ can be a thiazolidinedione or a glitazar.

The cells which are to be differentiated to a brown adipocyte phenotype according to the methods described herein can be non-neuronal somatic cells, differentiated non-neuronal cells, fibroblasts, adipose-derived cells, adipose-derived stromal vascular cells, or stem or progenitor cells. Stem or progenitor cells useful in the methods described herein can include induced pluripotent stem cells, adipose-derived stem cells, adipose-derived mesenchymal stem cells, adipose progenitor cells, embryonic stem cells, and mesenchymal stem cells. In some embodiments, the cells are human cells.

In some embodiments, the brown adipocytes are differentiated in vitro. In some embodiments, the brown adipocytes are differentiated ex vivo.

In some embodiments, the rate of differentiation to brown adipocytes is at least 80%.

In some embodiments, the step of providing a population of cells can include inducing a population of pluripotent stem cells to differentiate to a mesenchymal stem cell phenotype.

In another aspect, the invention is directed to a method for promoting the differentiation of pluripotent stem cells into brown adipocytes comprising, (a) differentiating pluripotent stem cells into mesenchymal stem cells, (b) contacting the mesenchymal stem cells with at least one agent that increases the level or activity of PPARγ2 and C/EBPβ and (c) culturing the cells under conditions favorable for the differentiation into brown adipocytes. The method does not comprise contacting the cells with an agent that increases the level or activity of PRDM16.

In a further aspect, the invention is directed to a method for screening for agents that increase the development of brown adipocytes comprising, (a) contacting cells with at least one agent that increases the level or activity of PPARγ2 and C/EBPβ, (b) contacting the cells with an additional candidate agent and (c) culturing the cells under conditions favorable for differentiation into brown adipocytes. A candidate agent is identified as an agent that increases the development of brown adipocytes if the rate of proliferation or rate of differentiation of brown adipocytes is higher in the presence of the candidate agent.

In another aspect, the invention is directed to a method for screening for agents that increase the activity of brown adipocytes comprising, (a) contacting cells with at least one agent that increases the level or activity of PPARγ2 and C/EBPβ, (b) culturing the cells under conditions favorable for differentiation into brown adipocytes and (c) contacting the brown adipocytes with a candidate agent. A candidate agent is identified as an agent that increases the activity of brown adipocytes if a measure of brown adipocyte activity is higher in the presence of the candidate agent.

In some embodiments, the measure of brown adipocyte activity is the generation of heat. In some embodiments, the measure of brown adipocyte activity is the rate of growth or proliferation of the adipocytes. In some embodiments, the measure of brown adipocyte activity can be the expression of brown adipocyte marker genes, measurement of mitochondrial number and activity and/or glycerol release.

In a further aspect, the invention is directed to a method of providing brown adipocytes to a subject in need thereof comprising (a) differentiating brown adipocytes from cells ex vivo as described herein and (b) transplanting the brown adipocytes so differentiated into the subject. In some embodiments, the cells are autologous.

In another aspect, the invention is directed to a kit for promoting the differentiation of cells into brown adipocytes comprising (a) at least one agent that increases the level or activity of PPARγ2 and C/EBPβ and (b) optionally, a population of cells. The kit does not comprise an agent which increases the level of PRDM16.

In one aspect, the invention is directed to brown adipocytes obtained in accordance with the methods described herein. In one aspect, the invention is directed to the use of brown adipocytes, obtained in accordance with the methods described herein, in therapy.

### BRIEF DESCRIPTION OF THE DRAWINGS

FIGS. 1A-1C depict the scheme of the experiments described in the Examples Section and characterization of the mesenchymal progenitor cells (MPCs). FIG. 1A depicts the experimental scheme for the differentiation of adipose-derived stromal-vascular cells (ADSVCs) and human pluripotent stem cells (hPSCs) into white and brown adipocytes. ADSVCs were isolated from human primary adipose tissue and then either not infected (“unprogrammed”) or transduced with lentivirus carrying an inducible PPARG2 cDNA transgene (lenti-PPARG2) and a second lentivirus constitutively expressing the reverse tetracycline trans-activator domain (lenti-rtTA M2) (“programmed”). hPSCs were differentiated as embryoid bodies (EBs) and then replated and passaged to generate mesenchymal progenitor cells (MPCs). MPCs were either unprogrammed or programmed with lenti-rtTA in combination with lenti-PPARG2 by itself, or with lenti-PPARG2 and lenti-CEBPB (PPARG2-CEBPB) or lenti-PPARG2, lenti-CEBPB and lenti-PRDM16 (PPARG2-CEBPB-PRDM16) respectively. Both unprogrammed and programmed ADSVCs and MPCs were cultured in media containing adipogenic factors (insulin, dexamethasone, and rosiglitazone) and doxycycline. For white fat differentiation doxycycline was withdrawn after 16 days and the cells were maintained in adipogenic media for at least 5 additional days prior to analysis. Four hPSC lines were used—two human embryonic stem cell (hESC) lines, HUES 8 and 9, and two induced pluripotent stem cell (iPSC) clones generated by reprogramming BJ fibroblasts with modified ribonucleic acids, BJ RiPSC #1.1. For brown fat differentiation doxycycline was withdrawn after 14 days and the cells were maintained in adipogenic media for an additional 7 days prior to analysis. FIG. 1B depicts the characterization of MPCs. The top panel depicts the results of the flow cytometry of the human pluripotent stem cell lines BJ RiPS #1.1 and HUES 9 as well as MPCs derived from these lines. Cells were stained for the surface antigens Stro1,CD105, CD73, CD44, CD29 and CD4. Numbers represent the percentage of positive cells. Where appropriate, positive stainings were distinguished as high or low expression groups (high expression=shaded boxes on the “BJ RIPS #1.1 MPC p6” and HUES 9 MPC p7” lines; low expression=shaded boxes on the “BJ RIPS #1.1 p15” and “HUES 9 p30” lines). The bottom panel depicts flow cytometry for the surface antigens Stro1, CD105, CD73, CD44, and CD29 presented as histograms. BJ RiPS #1.1 p15 (top traces in each box), BJ RiPS #1.1 MPC p6 (2^{nd} trace from the top in each box), HUES 9 p30 (2^{nd} trace from the bottom in each box) and HUES 9 MPC p7 (bottom trace in each box) The x-axis indicates the relative fluorescent intensity from 10 to 100.000 on a logarithmic scale. The y-axis represents the percentage of cells. FIG. 1C depicts doxycycline-inducible expression of PPARG2 and EGFP. Left: Quantitative RT-PCR assays for viral PPARG2 cDNA expression normalized to HPRT: BJ RiPS MPCs transduced with lenti-rtTA only (control); BJ RiPS MPCs transduced with lenti-rtTA and lenti-PPARG2 cultured in the absence (−DOX) or presence (+DOX) of doxycycline. Right: BJ RiPS MPCs transduced with lenti-rtTA and doxycycline-inducible EGFP virus and cultured either in the absence (−DOX; top panels) or presence (+DOX; bottom panels) of doxycycline.

FIGS. 2A-2D depict efficiency calculations and morphological characterization of differentiation. FIG. 2A depicts the efficiency of white adipocyte differentiation. Table: Efficiency of white adipocyte formation from cells in adipogenic media alone (untransduced) or PPARG2-programmed, differentiated cells (PPARG2) as determined by a ratio of HOECHST-positive and CEBPA-positive nuclei. Image panels: Representative images of HOECHST-stained (top left panel) and CEBPA-stained (lower left panel) nuclei from HUES 9 MPCs programmed with PPARG2 (100×magnification). The right panels illustrate the threshold assignment of positive nuclei and counting performed by the Image J analysis software. FIG. 2B depicts the results of programming human pluripotent stem cells into white adipocytes. HUES 8-derived MPCs were differentiated with adipogenic media alone (top panels; untransduced) or in combination with exogenous PPARG2 expression (upper panels; +PPARG2). Shown from left to right: brightfield images illustrating the morphology of immature (top panel) and mature (lower panel) white adipocytes; fluorescent images of corresponding immunostains with antibodies against the adipocyte marker protein FABP4 and the neutral lipid dye BODIPY; all cells were co-stained with HOECHST to identify nuclei (100×magnification). Staining appears as a grey color. FIG. 2C demonstrates that hPSC-derived white adipocytes express endogenous CEBPA and PPARG2. BJ RiPSCs derived MPCs were differentiated and programmed with exogenous PPARG2 expression for 16 days and, 5 days after withdrawal of doxycycline, stained with antibodies against CEBPA (lower left panel) or PPARG2 (lower right panel). All cells were also stained with the neutral lipid dye BODIPY (both lower panels, 100×magnification). Staining appears as a grey color. The upper panels show corresponding brightfield images. FIG. 2D demonstrates that hPSC-derived brown adipocytes express UCP1 and can be efficiently labeled with Mitotracker. BJ RiPSCs derived MPCs were differentiated with adipogenic media alone (top panels; untransduced) or programmed with either exogenous PPARG2+CEBPB (middle panel) or exogenous PPARG2+CEBPB+PRDM16 (lower panel) expression for 14 days and, 7 days after withdrawal of doxycycline, labeled with Mitotracker or were stained with antibodies against UCP1. Staining appears as a grey color. The left panel show corresponding brightfield images (all images 200x magnification).

FIGS. 3A-3B depict the molecular characterization of differentiated cells. FIG. 3A demonstrates that hPSC-derived white adipocytes express mature marker genes. Quantitative RT-PCR assays were performed for adipocyte marker genes PPARG2, CEBPA, FABP4, ADIPOQ, HSL, and LPL. Expression values represent three biological replicates and are shown as relative to HPRT expression in each sample. White bars represent cells that were not exposed to adipogenic media (undifferentiated); grey bars represent cells that were exposed to adipogenic media but not transduced with lenti-PPARG2 (−PPARG2);) black bars represent cells that were exposed to adipogenic media and transduced with lenti-PPARG2 (+PPARG2). P values represent two-tailed Student t-tests between −PPARG2 and +PPARG2 expression values for each cell line. *P<0.05; **P<0.01. P values shown under each gene name represent ANOVA analyses among all expression values (including data in FIGS. 5A-5E) for −PPARG2 and +PPARG2 cell lines. FIG. 3B depicts the comparison of hPSC-derived white adipocytes and brown adipocytes. Quantitative RT-PCR assays were performed for a range of white or brown adipocyte marker genes PPARG2, PGC1a, FABP4, ADIPOQ, HSL, LPL, CYTOCHROME C1, ELOL3 and UCP1. Expression values represent three biological replicates and are shown relative to HPRT expression and relative to with the lenti-PPARG2 condition set as 1. White bars represent cells that were differentiated with adipogenic media alone (untransduced); black bars represent cells that were exposed to adipogenic media and transduced with lenti-PPARG2 (+PPARG2); grey bars represent cells that were exposed to adipogenic media and transduced with lenti-PPARG2 and lenti-CEBPB (+PPARG2-CEBPB, light grey bars) or with lenti-PPARG2, lenti-CEBPB and lenti-PRDM16 (+PPARG2-CEBPB-PRDM16, dark grey bars) respectively. All experiments were performed with BJ RiPSCs derived MPCs. P values represent two-tailed Student t-tests between the PPARG2 and (PPARG2-CEBPB) or (PPARG2-CEBPB-PRDM16) setups respectively. Values for each cell line. *P<0.05; **P<0.01.

FIGS. 4A-4E depicts functional characterization hPSC-derived white adipocytes. FIG. 4A demonstrates that hPSC-derived adipocytes perform lipolysis. Glycerol was measured in the supernatant of ADSVCs and HUES 9-derived MPCs that were either not exposed to adipogenic media (undifferentiated) or exposed to adipogenic media without (−PPARG2) or with exogenous PPARG2 expression (+PPARG2) followed by either treatment with (+iso) or without (−iso) Isoproterenol. The quantity of glycerol released (in μg) was normalized to the total amount of protein (in mg) for each sample. **P<0.01. FIG. 4B demonstrates that hPSC-derived adipocytes secrete adiponectin. Enzyme-linked immunosorbent assay (ELISA) for adiponectin in the supernatant of cells exposed to adipogenic media and either not transduced with lenti-PPARG2 (−PPARG2) or transduced with lenti-PPARG2 (+PPARG2). Experiments performed as biological triplicates, with the exception of BJ RiPS MPCs. *P<0.05; **P<0.01. FIG. 4C depicts lipid profiling of ADSVC- and hPSC-derived adipocytes. The cellular lipid content of PPARG2-programmed ADSVCs, HUES 9-derived MPCs, and BJ RiPS-derived MPCs was analyzed using a tandem mass spectroscopy lipidomics platform and compared to the lipid content of primary adipose tissue. Shown are the relative abundances of several long-chain triacylglyceride species in each cell type. The x-axis denotes the total number of carbon atoms in the fatty-acid chains:unsaturated bonds. The y-axis represents the relative abundance of each lipid analyte. FIG. 4D depicts the attenuation of insulin induced Ser 473 Phosphorylation on AKT by FFAs. BJ RiPS MPC derived adipocytes were treated using either insulin alone, BSA-bound FFAs or with both. Phosphorylation of AKT was determined in the whole cell lysate by immunoblotting with the phospho-specific AKT (S473) antibody. FIG. 4E depicts glucose uptake in BJ RiPS MPC derived adipocytes as assessed by the transport of [3H]-2-deoxy-D-glucose upon insulin stimulation. MPC were exposed to adipogenic media without (−PPARG2) or with exogenous PPARG2 expression (+PPARG2) followed by either treatment with (+insulin) or without (−insulin) insulin. The quantity of [3H]-2-deoxy-D-glucose transported into the cells was normalized to CytoB and the results are shown as cpm *P<0.05, **P<0.01.

FIGS. 5A-5C depicts the functional characterization of hPSC-derived brown adipocytes. FIG. 5A depicts a glycerol release assay with hPSC-derived brown and white adipocytes. Glycerol was measured in the supernatant of HUES9-derived MPCs that were differentiated with adipogenic media alone (untransduced, white bars), with exogenous PPARG2 expression (+PPARG2, black), with expression of a combination of lenti-PPARG2 and lenti-CEBPB (+PPARG-CEBPB, light grey bars) or with the combination of lenti-PPARG2, lenti-CEBPB and lenti-PRDM16 (+PPARG-CEBPB-PRDM16, dark grey bars) are represented. After differentiation the cells were measured at basal level and after exposure to Forskolin (+FSK). The quantity of released glycerol (in μg) was normalized to the total amount of protein (in mg) for each sample. (Student's t-test**P<0.01). FIGS. 5B-5C depict comparison of the oxygen consumption rate (OCR) and extra-cellular acidification rate (ECAR) of hPSC-derived brown and white adipocytes. The OCR and ECAR were determined using no cells and cells differentiated with adipogenic media alone (untransduced) as controls. The OCR and ECAR of cells in which PPARG2 was exogenous expressed (+PPARG2), cells that were transduced with lenti-PPARG2 and lenti-CEBPB (+PPARG2-CEBPB), and lenti-PPARG2, lenti-CEBPB and lenti-PRDM16 (+PPARG2-CEBPB-PRDM16) transduced cells are also depicted. The OCR and ECAR were measured over time in approximately 5 minute intervals. The first two measurements were conducted to establish a baseline rate, followed by three measurements after the addition of oligomycin, an ATPase inhibitor (I). By uncoupling the proton gradient with carbonyl cyanide m-chlorophenyl hydrazone (CCCP) the maximum OCR and ECAR were determined over the next 3 time intervals (II). Finally at two timepoints measurements were conducted after inhibition of the mitochondrial respiratory chain with Antimycin (III). All experiments were conducted with BJ RiPSCs derived MPCs. P values represent two-tailed Student t-tests between untransduced and transduced cells. Values for each cell line. *P<0.01; **P<0.001.

FIGS. 6A-6B depict the transplantation of hPSC-derived white and brown adipocytes. FIG. 6A depicts HUES 9 derived MPCs which were transduced with lenti-PPARG2 and after 2 weeks of differentiation harvested and injected subcutaneously into a RAG2;IL2γC double knockout mice. 4-6 weeks after the injection prominent cell growth was visible at the injection site. This fat pad was harvested, sectioned and stained. Top panel: Brightfield morphology of the transplant sections (left). White square indicates zoom area. Zoomed brightfield image of the transplant section (middle). Immunohistochemistry overlay of stainings for nuclear marker Dapi, staining with a human specific nuclei marker MAB1281and staining with antibody against CEBPA (positive staining appears as a grey color). Bottom panel from left to right: Staining with nuclear marker Dapi (left), staining with a human specific nuclei marker MAB 1281 (middle) and staining with antibody against CEBPA (right). FIG. 6B depicts HUES 9 derived MPCs which were transduced with a combination of lenti-PPARG2, lenti-CEBPB and lenti-PRDM16 and transplanted and harvested as described above. Specimens were sectioned and adjacent slides were stained with UCP1 and MAB1261 a human specific nuclei marker (all images 200×magnification).

FIGS. 7A-7D depicts MPC derivation. FIG. 7A depicts the experimental scheme including the timeline and steps necessary to derive mesenchymal progenitor cells (MPCs) from human) pluripotent stem cells (hPSCs). hPSCs were differentiated as embryoid bodies (EBs) in suspension culture. Formed EBs were replated on dishes, and outgrowing differentiated cells were passaged several times and were analyzed for expression of mesenchymal surface markers and subsequently used in differentiation experiments. FIG. 7B depicts brightfield images showing different stages during the derivation of MPCs. From left to right: embryoid bodies 7 days after placement of pluripotent cells into suspension culture in ultra-low-attachment dishes; replated embryoid bodies at day 12 (MPC passage 0) just before cells were passaged; MPC passage 3, day 25 after EB formation, showing a homogenous population of cells with the appearance of fibroblasts. FIG. 7C depicts qRT-PCR characterization of MPCs over time. Cells were analyzed as hPSCs, 4 day old EBs, and after various passages (passage 0, 1, 3, 7) for the mesendoderm marker GSC (top), the mesoderm marker TBX (middle) and the pluripotency marker NANOG. (n=3, relative expression to HPRT, largest expression set to 1). FIG. 7D depicts a list of Oligonucleotides used to perform quantitative RT-PCR reactions in this study. FIG. 7D discloses the “forward” sequences as SEQ ID NOS 20-36, respectively, in order of appearance and the “reverse” sequences as SEQ ID NOS 37-53, respectively, in order of appearance.

FIGS. 8A-8D depicts flow analysis of MPC surface antigens. FIG. 8A depicts flow sytometry run 1, for the surface antigens CD105, CD73, CD44, CD 19 and CD4 presented as histograms. HUES 1 p39 (top trace), Human Monocytes (second trace from top), HUES 2 MPC p5 (third trace from top), HUES 8 MPC p4 (fourth trace from top), BJ RiPS #1.1 MPC p11 (fifth trace from top), ADSVC p7 (bottom trace). The x-axis indicates the relative fluorescent intensity of the indicated antibody from 10 to 400.000 on a logarithmic scale. The y-axis represents the percentage of cells. FIG. 8B depicts flow sytometry for the surface antigens Strol, CD105, CD73, CD44, CD29, CD 4 and unstained controls presented as histograms. BJ RiPS #1.1p15 (top trace), BJ RiPS #1.1 MPC p6 (second trace from top), HUES 9 p30 (second trace from bottom) and HUES 9 MPC p7 (bottom trace) The x-axis indicates the relative fluorescent intensity from 10 to 100.000 on a logarithmic scale. The y-axis represents the percentage of cells. FIG. 8C depicts a table showing the results of the two flow cytometry experiments. Numbers represent the percentage of positive cells. If appropriate, positive stainings were distinguished as high or low expression groups (high expression=shaded boxes in the “BJ RIPS #1.1 MPC p6” and “HUES 9 MPC p7” lines; low expression=shaded boxes in the “BJ RIPS #1.1 p15” and “HUES 9 p30” lines. FIG. 8D depicts Flow Analysis of MPC surface antigens. The gating tree was set as follows. Left column: FSC/SSC represents the distribution of cells in the light scatter based on size and intracellular composition, (respectively) to right column: live gate (PE, PE-Cy5, FITC, which represents the fraction of the positive stained cells (Strol, CD29, CD105, CD73, CD44 and CD4).

FIG. 9 depicts an array tree cluster which demonstrates that MPCs have the molecular signature of primary mesenchymal stem cell lines. GEO entries on the Affymetrix Human Genome U133 Plus 2.0 platform were selected randomly from a pool of entries that contained the key words “MSCs” and “hESCs” along with several studies focused on various tissue and cell types. All array data was processed and normalized using the RMA feature in the Bioconductor “affy” package in R. Probesets were mapped to and median collapsed onto HUGO gene symbol identifiers and median centered by array.

FIGS. 10A-10F depict MPC differentiation into osteoblasts and chondrocytes; differentiation potential of different ADSVC lines. FIG. 10A depicts MPCs which were differentiated into osteoblasts. To confirm the osteoblast differentiation, the top panel shows from left to right: an alizarin red staining at 100 fold, an alizarin red staining at 200 fold magnification and immunocytochemistry for alkaline phosphatase at 200 fold magnification. FIG. 10B demonstrates the confirmation of chondrocyte differentiation by staining sectioned MSC microspheres with hematoxylin and eosin (left), tolouidine blue was used to stain glycosaminoglycans (middle) and immunohistochemistry against chondrocyte specific Collagen II (right and bottom). FIGS. 10C-10F depict two distinct ADSVC lines which were differentiated for 21 days (16 days with doxycycline followed by 5 days without doxycycline). Both lines were either untransduced (unprogrammed) or transduced (programmed) with Lenti-PPARG and Lenti-rtTA. Experiments were conducted in biological triplicates. Shown are representative images of Oil-Red-O stained adipocytes. From top to bottom: FIG. 10C) programmed ADSVC [1] cells; FIG. 10D) unprogrammed ADSVC [1] control cells; FIG. 10E) programmed ADSVC [2] cells; FIG. 10F) unprogrammed ADSVC [2] control cells.

FIGS. 11A-11F depict the determination of Lentiviral titer; long-term culture of MPC derived adipocytes after doxycycline withdrawal; controlled expression of PRDM16 and CEBPB in MPCs; UCP1 screen. FIG. 11A depicts the determination of lentiviral titer. In one well of a 12-well dish, approximately 50,000 BJ RiPS MPCs were transduced with a fixed volume of Lenti-rtTA viral supernatant and declining volumes of Lenti-EGFP viral supernatant. Cells were induced with doxycyline for 48 hours, and GFP fluorescence pictures were acquired using fixed-exposure settings (fluorescence appears as grey color). The viral copy number for the Lenti-EGFP virus in the supernatants of virus preparations was determined using the Lenti-X qRT-PCR method. In FIG. 11B, using 500 μl of Lenti-rtTA and 500 μl of Lenti-EGFP viral supernatants, 50,000 BJ RiPS MPCs plated in wells of a 12-well dish were transduced and exposed to doxycyline for 48 hours. Percentages of GFP-positive cells were determined by counting cells from three independent experiments. FIG. 11C depicts in vitro programming of hPSCs into adipocytes. PPARG2-programmed BJ RiPS MPCs were differentiated with adipogenic media in the presence of doxycycline for 16 days, followed by an additional 20 days of differentiation in the absence of doxycycline. Top panel left: brightfield image showing the morphology of the differentiated mature adipocytes. Top panel right: staining with HOECHST dye. Bottom panel left: Immunostaining for CEBPA. Bottom panel right: Neutral lipid using BODIPY dye. FIG. 11D depicts doxycycline-inducible expression of PPARG2. Quantitative RT-PCR for viral PPARG2 cDNA expression normalized to HPRT, except for primary fat for which endogenous PPARG expression normalized to HPRT is shown. From left to right: BJ RiPS MPCs transduced with Lenti-rtTA only (control); BJ RiPS MPCs transduced with Lenti-rtTA and Lenti-PPARG2 cultured in the absence (−DOX) or presence (+DOX) of doxycycline for 48 hours; BJ RiPS MPCs transduced with Lenti-rtTA and Lenti-PPARG2 and differentiated for 16 days with doxycycline followed by 5 days without doxycycline; primary fat. FIG. 11E depicts MPCs which were transduced with Lenti-rtTA and Lenti-PRDM16 (top panel), or Lenti-rtTA and Lenti-CEBPB (bottom panel) respectively. The cells were either left untreated (left panel) or exposed to 700 ng/ml of doxycycline for 48 h (right panel). Immunostaining was performed using the antibodies against PRDM16 or CEBPB. (100×magnification). FIG. 11F depicts the results of combinations of Lenti-PPARG2, lenti-CEBPB and PRDM16 which were screened for the potential to induce brown fat differentiation. The cells were differentiated for 21 days (14 days with doxycycline followed by 7 days without doxycycline). Shown are the result of subsequent quantitative RT-PCR toward UCP1 as a brown adipocyte marker, ADIPONECTIN and CIDEC, both adipocyte markers. P values in the bar graphs represent two-tailed Student t-tests between the various programmed conditions and unprogrammed expression values. (n=3, relative expression to HPRT, normalized to 1, *P<0.05; **P<0.01).

FIGS. 12A-12B depict lipidomic profiling of ADSVC- and hPSC-derived adipocytes. The cellular lipid content of ADSVCs, HUES 9 MPCs, and BJ RiPS MPCs programmed into adipocytes with PPARG2 was analyzed using a tandem mass spectroscopy lipidomics platform and compared to primary adipose tissue. FIG. 12A depicts diacylglycerides of a size range between 32:2 to 36:1. Programmed cells (first series); unprogrammed cells (second series); undifferentiated cells (third series); adipocytes (fourth series). FIG. 12B depicts lysophosphatidylcholine lipids, part of the membrane lipids present in all cells. The displayed size range is between 16:1 and 22:6. Programmed cells (first series); unprogrammed cells second series); undifferentiated cells (third series).

FIGS. 13A-13F depicts quantitative RT-PCR analysis for adipogenic markers; timecourse of doxycycline withdrawal; leptin release; glycerol release after isopreterenol exposure; transplantation BAT. FIG. 13A depicts quantitative RT-PCR analysis of the expression of adipogenic marker genes: PPARG, CEBPA, FABP4, ADIPOQ, HSL, and LPL. The data represent three biological replicates and are shown as relative expression to the housekeeping gene HPRT. Shown in in the section between the y-axis and the first vertical line are unprogrammed, undifferentiated control cells. Shown in the section between the first and second vertical line are unprogrammed, differentiated cell lines. Shown in the section to the right of the second vertical line are programmed, differentiated cell lines. Shown in the last bar and, in some graphs, on a separate scale are gene expression levels in primary fat obtained from the pannus of a patient who underwent elective surgery. P values in the bar graphs represent two-tailed Student t-tests between programmed and unprogrammed expression values for each cell line. *P<0.05; **P<0.01. P values shown under each gene name represent ANOVA analyses among all expression values for the programmed and unprogrammed cell lines. FIG. 13B depicts hPSC-derived MPCs which were transduced with PPARG2 and adipogenic differentiation was initiated through administration of doxycycline in adipogenic medium. Doxycycline was withdrawn at indicated time points (X-axis). All cells were differentiated until day 21. qRT-PCR was performed for adipocyte marker genes HSL, ADIPOQ, FABP4. Expression values represent two biological replicates and are shown as relative to HPRT expression in each sample. Relative expression was set to 1. FIG. 13C depicts leptin release assay: Enzyme-linked immunosorbent assay (ELISA) for peptin in the supernatant of hPSC-derived PPARG2 programmed adipocytes exposed to adipogenic media. FIG. 13D depicts glycerol release after Isopreterenol exposure: hPSC-derived brown adipocytes respond to isopreterenol by releasing glycerol from the cells. Glycerol was measured in BJ RiPS-derived MPCs that were differentiated with adipogenic media alone (untransduced, white bars), with expression of a combination of (PPARG2-CEBPB) (light grey bars) or (PPARG2-CEBPB-PRDM16) (dark grey bars). The cells were measured at basal level (−iso) and after exposure to Isopreterenol (+iso). The quantity of released glycerol (in μg) was normalized to the total amount of protein (in mg) for each sample (Student's t-Test **P<0.01). FIG. 13E depicts immunostaining of primary mouse brown adipose tissue and human adipose tissue: i) Mouse interscapular BAT were used as MAB1281 negative and UCP1 positive control. (pictures taken using confocal microscopy, with 400 fold magnification+confocal digital zoom). ii) Brightfield picture (left), UCP1 immunostain (middle) and overlay of UCP1 and HOECHST stain of primary mouse BAT (magnification 200x). iii) Brightfield morphology of human primary fat (magnification 200x). FIG. 13F depicts quantitative RT-PCR analysis for the expression of PRDM16 in pluripotent cells (black), untransduced MPCs (white) or with expression of a combination of (PPARG2-CEBPB) (light grey bars) or (PPARG2-CEBPB-PRDM16) (dark grey bars). Cells were collected at day 5 (under exposure to doxycycline) and day 25 (doxycycline withdrawn from media) and expression was measured using oligos detecting only endogenous expressed PRDM16 (top) and with oligos detecting endogenous and viral expression. The data is shown as relative expression to the housekeeping gene HPRT, with the untransduced control set as 1.

FIGS. 14A-14B depict vector maps of the lentiviral constructs. FIG. 14A depicts the TRE-PPARγ2 based on FUGW vector and FIG. 14B depicts the ubiquitin rtTA M2.

FIGS. 15A-15C depict micrographs showing results from viral efficiency assays. ADMSC were infected with supernatant eGFPrtTA M2 in a 1:2 ratio. Micrographs were generated 24 hours after doxycycline induction. FIG. 15A shows an overlay of FIGS. 15B and 15C. FIG. 15B shows cells in brightfield. FIG. 15C shows GFP expression (appears as a grey color).

FIG. 16 depicts micrographs showing results from the expression of combinations of various transcription factors as indicated in the top row: Pictures taken in 200x magnification. Shown are cells originated from the BJ RiPS line #1.1 differentiated for 21 days in adipogenic differentiation medium. The cells are shown in bright field exhibiting typical features of white adipose tissue in the PPARg control and brown adipose tissue in the PPARg, CEBPb and PRDM16 combinations. Nucleoli were stained with OAPI, Mitotracker was used for mitochondrial staining and an antibody fluorescence stain against UCP as a brown fat marker was performed. Positive staining appears as a grey color.

FIG. 17 depicts results of semi-quantitative reverse transcription PCR: Normalized to HPRT, p-values indicated. cDNA originated from CPCs generated from BJ RiPS#1.1 cells transduced with various transcription factors and differentiated as indicated in FIGS. 1A-1C for 21 days in adipogenic differentiation medium. ELOVL3 and UCP1 both brown fat markers, were expressed significantly higher in the PPARg, C/EBPb and PRDM16 combinations than in the PPARg and no virus control. In the PPARg white adipocyte control CIDEC a white adipocyte specific marker was significantly higher expressed than in the other cells.

FIG. 18 depicts the result of the functional characterization of the brown adipocyte-like cells via Glycerol release with and without Forskolin induction. WAT and BAT react to Forskolin induced cAMP influx with increased metabolic activity as indicated through Glycerol release.

FIG. 19 depicts the ORO staining of brown adipose tissue differentiated from BJ fibroblasts. Images are shown at 10×magnification. The left panel depicts cells transfected with empty vector controls and the right panel shows cells transfected with C/EBPβ and PPARγ2.

### DETAILED DESCRIPTION

Described herein are methods and compositions for promoting the differentiation of stem or precursor cells to brown adipocytes. Briefly, it has been discovered that treatments that increase the expression or activity of the transcription factors C/EBPβ and PPARγ2 can drive the differentiation of stem or precursor cells to a brown adipocyte phenotype. Importantly, and contrary to prevailing wisdom in the field, it has been determined that this differentiation does not require the expression or activity of the factor PRDM16. As such, methods described herein for promoting the differentiation of stem cells, or progenitor or precursor cells to brown adipocytes generally involve treating stem, precursor or progenitor cells with agents that increase the expression or activity of C/EBPβ and PPARγ2 polypeptides, without the need for agents that increase the expression or activity of a PRDM16 polypeptide.

Materials, procedures and considerations necessary to understand and use the disclosed methods, compositions and kits are described in the following, as are experimental results and non-limiting examples that demonstrate and illustrate various embodiments of the methods and compositions described.

### Definitions

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); The ELISA guidebook (Methods in molecular biology (149) by Crowther J. R. (2000); Fundamentals of RIA and Other Ligand Assays by Jeffrey Travis, 1979, Scientific Newsletters; Immunology by Werner Luttmann, published by Elsevier, 2006. Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

The terms “decrease,” “reduce,” “reduced”, “reduction” , “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level (e.g. the absence of a given treatment) and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level.

The terms “increased”,“increase” or “enhance” or “activate” or “promote” are all used herein to generally mean an increase by a statically significant amount; for the avoidance of any doubt, the terms “increased”, “increase” or “enhance” or “activate” or “promote” means an increase of at least 10% as compared to a reference level, for example an increase of at least about 20%, or at least about 30%, or at least about 40%, or at least about 50%, or at least about 60%, or at least about 70%, or at least about 80%, or at least about 90% or, up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of “promoting differentiation”, the reference level can be the extent of differentiation prior to treatment or in the absence of treatment according to the methods described herein.

As used herein, the terms “treat,” “treatment,” “treating,” or “amelioration” when used in reference to a disease, disorder or medical condition, refer to therapeutic treatments for a condition where the subject is in need of more brown adipocytes, wherein the object is to reverse, alleviate, ameliorate, inhibit, slow down or stop the progression or severity of a symptom or condition. The term “treating” includes reducing or alleviating at least one adverse effect or symptom of a condition. In the case of obesity or being overweight, the adverse effect includes not only clinical symptoms or markers of obesity-related disease, but also aesthetic indicators, such that a non-obese, but overweight individual's desire for weight loss or lower BMI is encompassed as a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), decrease in BMI, delay or slowing of the clinical progression of a condition, and amelioration or palliation of a condition.

As used herein, the phrase “therapeutically effective amount”, “effective amount” or “effective dose” refers to an amount that provides a therapeutic or aesthetic benefit in the treatment, prevention, or management of a higher than desired BMI or an associated condition, e.g. an amount that provides a statistically significant decrease in at least one symptom, sign, or marker of a higher than desired BMI or an associated condition. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefitrisk ratio.

As used herein, the term “pharmaceutical composition” refers to the active agent in combination with a pharmaceutically acceptable carrier commonly used in the pharmaceutical industry.

As used herein, the term “administering,” refers to the placement of brown adipocytes as disclosed herein into a subject by a method or route which results in at least partial localization of the cells at a desired site. Pharmaceutical compositions comprising the brown adipocytes disclosed herein can be administered by any appropriate route which results in an effective treatment in the subject.

The term “isolated” as used herein in reference to cells refers to a cell that is mechanically separated from another group of cells with which they are normally associated in vivo. Examples of a group of cells are a developing cell mass, a cell culture, a cell line, and an animal. These examples are not meant to be limiting. Methods for isolating one or more cells from another group of cells are well known in the art. See, e.g., Culture of Animal Cells: a manual of basic techniques (3rd edition), 1994, R. I. Freshney (ed.), Wiley-Liss, Inc.; Cells: a laboratory manual (vol. 1), 1998, D. L. Spector, R. D. Goldman, L. A. Leinwand (eds.), Cold Spring Harbor Laboratory Press; Animal Cells: culture and media, 1994, D. C. Darling, S. J. Morgan, John Wiley and Sons, Ltd.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcriptiontranslation is considered “isolated.”

The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene.

The term “gene” means the nucleic acid sequence which is transcribed (DNA) to RNA in vitro or in vivo when operably linked to appropriate regulatory sequences. The gene can optionally include regions preceding and following the coding region, e.g. 5′ untranslated (5′ UTR) or “leader” sequences and 3′ UTR or “trailer” sequences, as well as intervening sequences (introns) between individual coding segments (exons).

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector can comprise additional elements, for example, the expression vector can have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the C/EBPβ and/or PPARγ2 gene in place of non-essential viral genes. The vector and/or particle can be utilized for the purpose of transferring any nucleic acids into cells either in vitro or in vivo. Numerous forms of viral vectors are known in the art.

The term “replication incompetent” when used in reference to a viral vector means the viral vector cannot further replicate and package its genomes. For example, when the cells of a subject are infected with replication incompetent recombinant adeno-associated virus (rAAV) virions, the heterologous (also known as transgene) gene is expressed in the patient's cells, but, the rAAV is replication defective e.g., lacks accessory genes that encode essential proteins for packaging the virus) and viral particles cannot be formed in the patient's cells.

The term “transduction” as used herein refers to the use of viral particles or viruses to introduce exogenous nucleic acids into a cell.

The term “transfection” as used herein to methods, such as chemical methods, to introduce exogenous nucleic acids, such as the nucleic acid sequences encoding an agent which increases the activity and/or level of PPARγ2 or C/EBPβ as described herein, into a cell. As used herein, the term transfection does not encompass viral-based methods of introducing exogenous nucleic acids into a cell. Methods of transfection include physical treatments (electroporation, nanoparticles, magnetofection), and chemical-based transfection methods. Chemical-based transfection methods include, but are not limited to those that use cyclodextrin, polymers, liposomes, nanoparticles, cationic lipids or mixtures thereof (e.g., DOPA, Lipofectamine and UptiFectin), and cationic polymers, such as DEAE-dextran or polyethylenimine.

The term “agent” refers generally to any entity which is normally not present or not present at the levels being administered to a cell, tissue or subject. An agent can be selected from a group comprising: polynucleotides; polypeptides and small molecules. A polynucleotide can be RNA or DNA, and can be single or double stranded, and can be selected from a group comprising: nucleic acids and nucleic acid analogues that encode a polypeptide. A polypeptide can be, but is not limited to, a naturally-occurring polypeptide, a mutated polypeptide or a fragment thereof that retains the function of interest.

Other than in the operating examples, or where otherwise indicated, all numbers expressing quantities of ingredients or reaction conditions used herein should be understood as modified in all instances by the term “about.” The term “about” when used in connection with percentages can mean±1%.

The term “statistically significant” or “significantly” refers to statistical significance and generally means a two standard deviation (2SD) difference, above or below a reference value. Additional definitions are provided in the text of individual sections below.

### Brown Adipose Tissue/Cells

The methods described herein permit the generation of brown adipocytes from stem, progenitor, or other cells. The characteristics of adipose cells in general, and brown adipocytes in particular, are described below. In one aspect, described herein are brown adipocytes obtained according to the methods described herein.

The term “adipose tissue” refers to loose connective tissue which stores fat and is composed of multiple cell that includes adipocytes and microvascular cells. Adipose tissue also comprises stem and progenitor cells and endothelial precursor cells. Two varieties of adipose tissue are found in mammals; white adipose tissue and brown adipose tissue.

As the name would imply, white adipose tissue comprises white adipocytes, which are adipocytes comprising a single large fat droplet, with a flattened nucleus located on the periphery of the cell. White adipose tissue functions to help maintain body temperature (via insulation) and to store energy in the Form of lipids. White adipose cells can be distinguished from precursor cells by the presence of a C/EBPα and PPARγ2-positive nucleus and high cytoplasmic levels of FABP4 as determined, e.g. by antibody staining as described in the Examples herein. Marker genes of white adipocytes are well known and include, by way of non-limiting example, lipoprotein lipase (LPL; NCBI Gene ID No. 4023), hormone-sensitive lipase (HSL; NCBI Gene ID No. 3991), adiponectin (ADIPOQ NCBI Gene ID No. 9370), FABP4 (NCBI Gene ID No. 2167), CEBPA (NCBI Gene ID No. 1050), and PPARG2 (NCBI Gene ID No. 5468; NCBI Reference Sequence NM_{—}015869), which can be assayed by quantitative RT-PCR as described in the Examples herein. The majority of the research into adipose tissue characterization, differentiation, disease, and metabolism has utilized white adipose tissue. When those in the field refer merely to research on “adipose tissue” or “adipose cells”, it is commonly understood that the experiments utilized white adipose tissue and/or cells. That is, reference to “adipose tissue” or “adipose cells,” without specific reference to “white adipose tissue/cells” or “brown adipose tissue/cells” is most often a reference to white adipose tissue or cells. That is, when brown adipose tissue/cells are discussed in the art, there is generally specific reference to “brown” adipose tissue or cells.

In contrast, brown adipose cells utilize the chemical energy in lipids and glucose to produce heat via non-shivering thermogenesis¹². As used herein “brown adipose cell” refers to an adipose cell comprising multiple lipid droplets throughout the cell, a rounded nucleus and a large number of mitochondria, which give the cells their distinctive brown color. Marker genes of brown adipocytes are well known and include, by way of non-limiting example, lipoprotein lipase (LPL), UCP1 (NCBI Gene ID No. 7350), ELOVL3 (NCBI Gene ID No. 83401), PGC1A (NCBI Gene ID No. 10891), CYC1 (NCBI Gene ID No. 1537), CEBPA, and PPARG2, which can be assayed by quantitative RT-PCR as described in the Examples herein. Brown adipocytes can be distinguished from white adipocytes by having high relative expression of, by way of non-limiting example, UCP1, ELOVL3, PGC1A, and CYC1 and low relative expression of, by way of non-limiting example, ADIPOO, HSL, and FABP4, while both cell types will display high levels of PPARγ2 and LPL expression.

The recent discovery that adult humans have functional brown adipose depots in inverse correlation to body mass index^{13,14,15,16}has fueled considerable interest in the therapeutic potential of brown adipocytes. Prior to the discoveries described herein, the transcription factors CCAAT/enhancer-binding protein beta (CEBPB) and PR domain containing 16 (PRDM16) were thought to function as key regulators of brown fat development and function^{17,18,19}.

### Cells

In certain embodiments, methods described herein comprise promoting the differentiation of brown adipocytes from a population of cells. In one embodiment the cells may be of non-embryonic origin, i.e. the cells are not isolated from an embryo. A cell type suitable for use in the methods of differentiation described herein can comprise any non-neuronal cell type although, in practice it can first be advantageous to re-program non-neuronal cells to a stem, progenitor or precursor cell phenotype. In some embodiments, a population of cells which is to be differentiated according to the methods described herein is comprised of a population of cell types in which at least 10% are non-neuronal cells, i.e. at least 10% of the cells present are not neuronal, 20% of the cells are not neuronal, 50% of the cells are not neuronal, 80% of the cells are not neuronal, 90% of the cells are not neuronal, or 95% or more of the cells are not neuronal. By way of non-limiting example, cells suitable for being differentiated into brown adipocytes according to the methods described herein can include fibroblasts, adipose-derived cells, adipose-derived stromal vascular cells, stem cells and/or progenitor cells. Methods of isolating a cell or population of cells suitable for differentiation to brown adipocytes are well known and will be readily apparent to those of ordinary skill in the art.

In some embodiments, cells suitable for being differentiated into brown adipocytes according to the methods described herein are part of the mesodermal cell lineage. As used herein, “the mesoderm” refers to the middle layer of the three germ layers which arise during development. The mesoderm gives rise to all connective tissues (except in the head and neck regions), all body musculature, blood, cardiovascular and lymphatic systems, most of the urogenital system and the lining of pericardial, pleural and peritoneal cavities. Included within the scope of the mesodermal lineage are precursor cells and terminally differentiated cells.

In some embodiments, cells suitable for being differentiated into brown adipocytes according to the methods described herein are cells of the mesenchymal cell lineage. As used herein, “mesenchymal” refers to cells and/or tissue that arise from the mesoderm and from which bone, cartilage, connective, circulatory and lymphatic tissues arise. Included within the scope of the mesenchymal lineage are precursor cells and terminally differentiated cells.

In some embodiments, cells suitable for being differentiated into brown adipocytes according to the methods described herein are cells of the stromal lineage. As used herein, “stromal” refers to a cell or tissue which provides a matrix or support for the cells performing a function of the organ involved, i.e. the parenchymal portion of an organ. Stromal tissue is typically connective tissue. Included within the definition of “stromal” are terminally differentiated cells as well as precursor cells that have the potential to differentiate into stromal cells and tissues.

In some embodiments, cells suitable for being differentiated into brown adipocytes according to the methods described herein are connective tissue cells. As used herein, “connective tissue” refers to those animal tissues that support organs, fill spaces between them, or perform mechanical functions such as connecting muscles to bone (tendons and ligaments) or providing low friction weighing surface as in articular cartilage. Connective tissues are characterized by their relatively avascular matrices and low cell densities. The most abundant connective tissues are the reticular stroma, muscle, adipose tissue, cartilage and bone. Further examples of connective tissue include, but are not limited to, mesenchyme, mucous connective, areolar (loose), elastic, or blood. Included within the definition of “connective tissue” are terminally differentiated cells as well as precursor cells that have the potential to differentiate into connective tissue cells and tissues.

As used herein, “fibroblast” refers to a flat elongated connective tissue cell with cytoplasmic processes at each end. A fibroblast can have a flat, oval, vesicular nucleus. Fibroblasts can be stellate (star-shaped) or spindle-shaped. Fibroblasts form the fibrous tissues in the body, including tendons, aponeuroses, supporting and binding tissues of all sorts. Fibroblasts are one example of a fully differentiated cell that can be differentiated to a brown adipocyte phenotype using the methods as described herein. Fibroblasts can be obtained from tissue samples, by way of non-limiting example, as described in U.S. Pat. No.7,816,133 or differentiated from stem or progenitor cells, by way of non-limiting example, as described in U.S. Pat. Publication No. 2005/0054100, which are incorporated by reference herein in their entirety.

As used herein, an “adipose-derived cell” is any cell type which is isolated from or is descended from a cell isolated from adipose tissue.

As used herein, an “adipose-derived stromal vascular cell” or “ADVSC” refers to all non-adipocyte cells obtained from the stromal vascular fraction (SVF) of adipose tissue (described herein below). ADVSCs can comprise somatic cells, differentiated cells, stem cells, and/or progenitor cells. ADVSCs can be isolated, by way of non-limiting example, by obtaining primary human adipose tissue from surgical waste of patients who have undergone elective surgery. Adipose tissue can be digested with Liberase™ (Roche) collagenase blend for one hour with gentle shaking at 37° C. Digested tissue can be forced through a 250 micron filter, and the filtrate collected and centrifuged. The resulting stromal vascular cell pellet can be washed twice with PBS and plated onto gelatin-coated plates (0.1%) in ADSVC growth media [DMEM, 10% FBS, 1% penicillinstreptomycin, and 2.5 ng/ml bFGF (Aldevron)]. ADSVCs can be passaged using trypsin upon reaching confluency.

As used herein, a “non neuronal cell” or a cell which is “not neuronal” refers to a cell lacking the characteristics of a neuron or neuronal cell, i.e. it is not a morphologic or functional unit of the brain, brainstem, spinal cord or peripheral nerves. A neuron is generally characterized as having a body in which the nucleus resides, dendrites, and an axon for transport of the nerve impulse.

The cells can be from any species suitable for use as a subject in the methods described herein. The cells can be animal cells, such as a mammal, (e.g., primate, rodent, or domestic animal). Examples of such mammals are a human, non-human primate, mouse, rat, dog, cat, horse, or cow. The cells can be of male or female origin. The cells can be primary cells obtained form an adult or an immature animal (e.g., embryo, fetus, infant or child).

In some embodiments, brown adipocytes differentiated according to the methods described herein are provided to a subject in need thereof. In some embodiments, the cells provided to a subject are autologous. In some embodiments, the cells provided to a subject are allogenic. In some embodiments, the cells provided to a subject are xenogenic.

As used herein, the term “population” when used to refer to cells used in the methods described herein refers to one or more cells, e.g. 1 cell, 100 cells, 1000 cells, 1×10⁵ cells, 1×10⁷ cells, 1×10⁹ cells or more. The population can be clonal in nature or it can have arisen from multiple individual parental cells. A population of cells can comprise substantially one type of cell (at least 90% of one cell type, i.e. 90%, 95%, 98%, or 99% or more of one cell type) or comprise 2 or more types of cells, e.g. pluripotent stem cells and mesenchymal stem cells.

### Stem and Progenitor Cells

In certain embodiments, the methods described herein comprise promoting the differentiation of brown adipocytes from a population of stem or progenitor cells. A population of stem or progenitor cells can comprise cells which are not stem or progenitor cells, for example, connective tissue cells or mature adipocytes can be present. A population of stem or progenitor cells comprises at least 50% stem or progenitor cells. In one embodiment the stem or progenitor cells are obtained without destruction of an embryo, as detailed below.

As used herein, the term “stem cell” refers to a cell in an undifferentiated or partially differentiated state that has the property of self-renewal and has the developmental potential to naturally differentiate into a more differentiated cell type, without a specific implied meaning regarding developmental potential (i.e., totipotent, pluripotent, multipotent, etc.). By self-renewal is meant that a stem cell is capable of proliferation and giving rise to more such stem cells, while maintaining its developmental potential. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating.

The term “somatic stem cell” is used herein to refer to any stem cell derived from non-embryonic tissue, including fetal, juvenile, and adult tissue. Natural somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, mesenchymal stem cells and hematopoietic stem cells.

In some embodiments, the stem or progenitor cells are pluripotent stem cells. In some embodiments, the stem or progenitor cells are totipotent stem cells.

In some embodiments, the stem or progenitor cells are embryonic stem cells. As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are totipotent cells derived from the early embryo or blastocyst. Embryonic stem cells can be obtained directly from suitable tissue, including, but not limited to human tissue, or from established embryonic cell lines. In one embodiment, embryonic stem cells are obtained as described by Thomson et al. (U.S. Pat. Nos. 5,843,780 and 6,200,806; Science 282:1145, 1998; Curr. Top. Dev. Biol. 38:133 ff, 1998; Proc. Natl. Acad. Sci. U.S.A. 92:7844, 1995 which are incorporated by reference herein in their entirety).

In some embodiments, the stem or progenitor cells are adult mesenchymal stem cells. As used herein “mesenchymal stem cells” (MSCs) refers to multipotent stem cells that can be differentiated into a variety of cell types including osteoblast, chondrocytes (cartilage cells), adipocyte (fat cells), myocytes, and β-pancreatic islet cells etc. Methods of isolating and identifying mesenchymal stem cells are known in the art and can include isolating mesenchymal stem cells from adipose tissue (see U.S. Pat. No. 5,486,359 U.S. Patent Publication 2009/0148419; 2011/0171726; which are incorporated by reference herein in their entirety). Accordingly, in some embodiments, the stem or progenitor cells are adipose-derived mesenchymal stem cells.

Phenotypically, MSCs express a number of markers, none of which, unfortunately, are specific to MSCs. It is generally agreed that adult human MSCs do not express the hematopoietic markers CD45, CD34, CD14, or CD11. They also do not express the costimulatory molecules CD80, CD86, or CD40 or the adhesion molecules CD31 (platelet/endothelial cell adhesion molecule [PECAM]-1), CD18 (leukocyte function-associated antigen-1 [LFA-1]), or CD56 (neuronal cell adhesion molecule-1), but they can express CD105 (SH2), CD73 (SH3/4), CD44, CD90 (Thy-1), CD71, and Stro-1 as well as the adhesion molecules CD106 (vascular cell adhesion molecule [VCAM]-1), CD166 (activated leukocyte cell adhesion molecule [ALCAM]), intercellular adhesion molecule (ICAM)-1, and CD29. In some embodiments, the presence of the markers Stro1, CD29, CD105, CD73 and CD44 and the absence of the markers CD19 and CD4 is used to identify cells as having an MSC phenotype. In contrast, hPSCs can be differentiated as lacking expression of CD73.

There are several reports that describe the isolation of both human and rodent MSCs using antibody selection based on the phenotype of MSCs. Some have used a method of negative selection to enrich for MSCs, whereby cells from the hematopoietic lineage are removed; others have used antibodies to positively select for MSCs.

MSCs from other species do not express all the same molecules as those on human cells; for example, although human and rat MSCs have been shown to be CD34−, some papers report variable expression of CD34 on murine MSCs/ It is generally accepted that all MSCs are devoid of the hematopoietic marker CD45 and the endothelial cell marker CD31. However, it is important to note that differences in cell surface expression of many markers can be influenced by factors secreted by accessory cells in the initial passages, and the in vitro expression of some markers by MSCs does not always correlate with their expression patterns in vivo.

There is also variable expression of many of the markers mentioned due to variation in tissue source, the method of isolation and culture, and species differences. For example, human adipose tissue is a source of multipotent stem cells called processed lipoaspirate (PLA) cells which, like bone marrow MSCs, can differentiate down several mesenchymal lineages in vitro. However, there are some differences in the expressions of particular markers: CD49d is expressed on PLA cells but not MSCs, and CD106 is expressed on MSCs but not PLA cells. CD106 on MSCs in bone marrow has been functionally associated with hematopoiesis, so the lack of CD106 expression on PLA cells is consistent with localization of these cells to a nonhematopoietic tissue.

MSCs can be differentiated from pluripotent stem cells as described in detail in the Examples herein. Briefly, hESCs and hiPSCs are cultured feeder free on Geltrex™ reduced growth factor basement membrane matrix (Invitrogen) in the chemically defined medium mTESR1 (Stem Cell Technologies). To induce differentiation of hESCs and hiPSCs into embryoid bodies (EBs), hPSCs are disaggregated with dispase into small clumps containing 5-10 cells and transferred to low-adhesion plastic 6-well dishes (Costar Ultra Low Attachment; Corning Life Sciences) in growth medium containing DMEM, 15% FBS, and 1% Glutamax™ L-glutamine replacement supplement. After 7 days in suspension culture, EBs are collected and replated on gelatin-coated 6-well dishes in medium containing DMEM, 10% FBS, and 1% Glutamax™. After cells reach confluency (in approximately 5 days) they are trypsinized (0.25% trypsin) and replated on cell culture dishes containing growth medium containing DMEM, 15% FBS, 1% Glutamax™, and 2.5 ng/ml bFGF (Aldevron).

In some embodiments, the stem or progenitor cells are adipose-derived stem cells (ADSCs). Markers for ADSC's include CD105 and CD73 and MSC's do not express the hematopoietic makers CD34, CD45, and CD14. ADSCs are also referred to in the art as, variously, preadipocytes, stromal cells, processed lipoaspirate cells, multipotent adipose-derived stem cells, and adipose-derived adult stem cells. As used herein, “adipose-derived stem cells” refers to multipotent stem cells isolated from adipose tissue which have osteogenic, adipogenic, myogenic, chrondrogenic, and neurogenic differentiation potential. ADSCs are a subpopulation of mesenchymal stem cells but can be differentiated from the general population of mesenchymal stem cells (MSCs) by the expression of CD49d and the absence of CD106 expression. ADSC's can be derived from umbilical cord tissue, Wharton's Jelly, pulp of deciduous baby teeth, amniotic fluid, adipose tissue or bone marrow. In some embodiments, ADSCs can be derived from adipose tissue, which can be harvested by direct excision or more commonly from lipoaspirate, the discarded tissue following liposuction surgery. The tissue can be washed and red blood cells removed. Digestion with collagenase can be performed and the tissue is centrifuged to obtain a cell pellet, known as the stromal vascular fraction (SVF). The SVF can contain, in addition to ADSCs, mesenchymal stem cells (MSCs) and endothelial cells. ADSCs can be purified from the SVF by, for example, prolonged culture of SVF, relying on the ability of ADSCs to outcompete other cell populations under the culture conditions over time. The number of stem cells present can be increased by subjecting the SVF to a 24-hour adhesion period before washing away nonadherent cells; the fraction of stem cells can be further increased by a forceful washing step at 1 hour into the 24-hour adhesion period. Alternatively, cell sorting (e.g. FACS) based on cell surface markers expressed by ADSC can permit purification of ADSCs from the SVF (see Miranville et al. Vascular Medicine 2004 110:349-355; Locke et al. Stem Cells 2011 29:404-411; Zuk et al. Molecular Biology of the Cell 2002 13:4279-4295; which are incorporated by reference herein in their entirety).

In some embodiments, the stem or progenitor cells are induced pluripotent stem cells (iPSCs). Stem cells can be naturally occurring cells isolated from an organism or maintained in culture or they can be induced stem cells. As used herein, “induced stem cells” refers to pluripotent stem cells which are created from differentiated cells by increasing the level or activity of certain factors known to promote dedifferentiation. For example, iPSCs can be obtained by overexpression of transcription factors such as Oct4, Sox2, c-Myc and Klf4 according to the methods described in Takahashi et al. (Cell, 126: 663-676, 2006). Other methods for producing iPSCs are described, for example, in Takahashi et al. Cell, 131: 861-872 2007 and Nakagawa et al. Nat. Biotechnol. 26: 101-106, 2008; which are incorporated by reference herein in their entirety. By way of non-limiting example, fibroblasts can be dedifferentiated to form iPSCs. Fully reprogrammed iPSCs can be identified by, for example, expression of the pluripotency markers ALPL, DNMT3B, DPPA4, FGF4, FOXD3, GDF3, LEFTY1(LEFTB), LEFTY2 (EBAF), NODAL, PODXL, TGDF1, UTF1, ZFP42 and Xist and the lack of expression of the spontaneous differentiation marker HAND1 and the somatic cell marker COLA1.

As used herein, “progenitor cells” refers to cells in an undifferentiated or partially differentiated state and that have the developmental potential to differentiate into at least one more differentiated phenotype, without a specific implied meaning regarding developmental potential (i.e., totipotent, pluripotent, multipotent, etc.) and that does not have the property of self-renewal. Accordingly, the term “progenitor cell” refers to any subset of cells that have the developmental potential, under particular circumstances, to differentiate to a more specialized or differentiated phenotype.

In some embodiments, the stem or progenitor cells are adipose progenitor cells. As used herein “adipose progenitor cells” refers to cells of adipose origin in an undifferentiated or partially differentiated state and that have the developmental potential to differentiate into brown and/or white adipose cells, without a specific implied meaning regarding developmental potential (i.e., totipotent, pluripotent, multipotent, etc.) and that does not have the property of self-renewal.

### Differentiation Factors

According to the methods described herein, the differentiation of brown adipocytes can be promoted by contacting a population of stem or progenitor cells with at least one agent that increases the level or activity of PPARγ2 and C/EBPβ.

As used herein, “increases the level or activity” means an increase of at least 10% in the level or activity of PPARγ2 and C/EBPβ, i.e. an increase of 10%, or 20%, or 40%, or 60%, or 80%, or 100%, or 200%, or 500% or more as compared to the level or activity in the absence of the agent. The level PPARγ2 and C/EBPβ can be measured as the level of mRNA encoding the polypeptide or the level of the polypeptide itself. The activity of PPARγ2 and C/EBPβ can be the level of activity of PPARγ2 and C/EBPβ as measured by any of the parameters and assays described herein in the sections entitled “PPARγ2 polypeptides” and “C/EBPβ polypeptides” below.

As used herein, “an agent which increases the level or activity” of a certain molecule can act directly or indirectly to increase the level or activity of the target molecule. As used herein, “an agonist which increases the level or activity” of a certain molecule acts directly and specifically to increase the level or activity of the target molecule and is, accordingly a subset of agents which increase the level or activity of target molecule. As used herein “an agent which increases the level or activity” of a certain molecule is understood to include agonists of that molecule. By way of non-limiting an example, a nucleic acid encoding a polypeptide “X” can be an agonist and an agent which increases the level or activity of X because it will cause an increase in, at least, the level of X present in the cell. It will also do this in a direct and specific manner. A nucleic acid encoding a polypeptide “Z” which is a positive transcriptional regulator of X and a third polypeptide Y, can be an agent which increases the level or activity of X because, by increasing the level of Z, it can increase the level of X. However, because it will not do so directly (e.g. it increases the level of Z, which then acts to increase the level of X) nor specifically (e.g. it also increases the level of Y), it is not an agonist of X.

In some embodiments, a population of stem or progenitor cells is contacted with an agent that increases the level or activity of PPARγ2 and C/EBPβ at a concentration of 0.005 μM to 50 μM. In some embodiments, a population of stem or progenitor cells is contacted with an agent that increases the level or activity of PPARγ2 and C/EBPβ at a concentration of 0.005 μM to 50 μM. In some embodiments, a population of stem or progenitor cells is contacted with an agent that increases the level or activity of PPARγ2 and C/EBPβ at a concentration of 0.1 μM to 1 μM.

### PPARγ2 Polyypeptides

PPARγ2 is a peroxisome proliferator-activated, nuclear hormone receptor-type transcription factor. Agents that increase the activity or level of a PPARγ2 polypeptide are used in the methods described herein, in conjunction with agents that increase the activity or level of a C/EBPβ polypeptide, to promote the differentiation of mammalian cells to a brown fat phenotype. In certain embodiments, PPARγ2 polypeptides themselves can be administered, e.g., either directly, or more often, via expression from a nucleic acid construct, in order to increase the level and activity of PPARγ2 in the cell targeted for differentiation. In other embodiments, for example, chemical agents and/or small molecules that increase either or both of the level or activity of a PPARγ2 polypeptide can be used. PPARγ2 is also referred to variously as “PPARG2” or “PPARgamma2.”

At a basic level, a PPARγ2 polypeptide is a polypeptide derived from or similar to PPARγ2 that retains the ability to direct or promote the differentiation of a stem or progenitor cell to a brown adipocyte phenotype in conjunction with an agent that increases the activity or level of a C/EBPβ polypeptide. Discussed below are the structural determinants identified for PPARγ2 polypeptides, as well as functions, including determinants of target gene binding and transactivation by PPARγ2 polypeptides useful according to the methods described herein.

PPARγ's include PPARγ1 and PPARγ2, which differ only in the inclusion of a 30 amino acid N-terminal extension on the PPARγ2 polypeptide. PPARγ1 does not direct the differentiation of stem, precursor, or other cells to brown adipocytes. Thus, the 30 amino acid N-terminal extension (SEQ ID NO: 09) of PPARγ2 relative to PPARγ1 is important for the function of PPARγ2 polypeptides useful in the methods described herein. PPARγ2 is the nutritionally-regulated form of PPARy (see, e.g., Vidal-Puig et al., J Clin. Invest. 97: 2553-2561 (1996)), and is expressed primarily in adipocytes, whereas PPARγ1 is expressed in a number of other cell types. Exemplary reference sequences are the human PPARγ2 mRNA (SEQ ID NO: 01; NCBI Reference Sequence: NM_{—}015869) and protein sequences (SEQ ID NO: 02; NCBI Reference Sequence: NP_{—}056953). One of ordinary skill in the art will recognize that sequence differences will exist due to allelic variation, and will also recognize that other animals, particularly other mammals, have corresponding PPARs, which have been identified or can be readily identified using sequence alignment and confirmation of activity.

Naturally-occurring PPARγ2 polypeptides generally comprise modular structural domains including (a) a N-terminal region with an Activation Function-1 (AF1) ligand independent transactivation domain, (b) a DNA binding domain with twin zinc finger DNA binding motifs, (c) hinge region, (d) a ligand-binding domain and (e) an Activation Function-2 (AF2) ligand-dependent transactivation domain at the extreme C terminus (See Zoete et al. Biochim Biophys Acta, 2007 1771:915-25, and Kilroy et al., Obesity 17: 665-673 (2009), each of which is incorporated by reference herein in its entirety. The N-terminal (AF-1) region corresponds to amino acids 30-140 of SEQ ID NO: 02; the DNA binding domain corresponds to amino acids 140-205 of SEQ ID NO: 02; the hinge domain corresponds to amino acids 205-281 of SEQ ID NO: 02; the ligand-binding domain corresponds to amino acids 281-495 of SEQ ID NO: 02; and the ligand-binding/activation function (AF2) ligand-dependent transactivation domain corresponds to amino acids 495-505 of SEQ ID NO: 02. PPARγ2 polypeptides useful according to the methods described herein include naturally-occurring mammalian PPARγ2 polypeptides including, but not limited to human PPARγ2 having the amino acid sequence at GenBank accession No. NP_{—}056953 (SEQ ID NO: 02), murine PPARγ2 having the amino acid sequence at GenBank accession No. NP_{—}035276 (SEQ ID NO: 10), as well as rat PPARγ2 having the amino acid sequence at GenBank accession No. NP_{—}037256 (SEQ ID NO: 11).

At a minimum, a “PPARγ2 polypeptide” as the term is used herein, can bind the regulatory region of a PPARγ2-responsive target gene (i.e., a target gene transactivated by a naturally-occurring PPARγ2 polypeptide) and transactivate (that is, up-regulate) the expression of that gene. Thus, “PPARγ2 activity” refers to the sequence-specific transactivation of a gene that is transactivated by a naturally-occurring, wild-type PPARγ2 polypeptide. It is preferred, but not required, as discussed below, that such transactivation is ligand-dependent.

PPARγ2 polypeptides useful in methods as described herein comprise a DNA binding domain that binds to one or more DNA sequences bound by wild-type, naturally-occurring mammalian PPARγ2 polypeptides. A non-limiting example of a consensus sequence for PPAR DNA-binding activity is AGGTCANAGGTCA (SEQ ID NO: 14). Non-limiting examples of consensus sequences for PPARγ DNA-binding are CAAAACTAGGTCAAAGGTCA (SEQ ID NO: 15); AGGNCAAAGGTCA (SEQ ID NO: 16); AGGTCA; and ANTGGGNCAAAGGTCA (SEQ ID NO: 17) (see Schmidt et al. BMC Genomics 2011 12:152; Hamza et al. PLoS One 2009 4:e4907; and Siersbaek et al. FEBS Letters 2010 584:3242-3249. It is preferred that the PPARγ2 polypeptide includes a twin zinc finger DNA binding domain that corresponds, for example, to the DNA binding domain of the PPARγ2 described by Zoete et al. At a minimum, a PPARγ2 polypeptide as described herein binds to the PPARγ2-sensitive promoter of the fatty acid binding protein 4 (FABP4) gene (NCBI Gene ID No 2167).

Zinc-finger DNA binding domains are well known in the art, and the structural determinants within them that provide for specificity for a given DNA sequence are apparent to those of skill in the art.

PPARγ2 polypeptides useful in methods as described herein comprise a transactivation domain (or domains) that, at a minimum, transactivates expression from a PPARγ2 sensitive reporter gene construct, such as the PPARγ2 -sensitive reporter gene construct pPPREx3TK-luciferase described by Floyd et al., Obesity Res. 12: 921-928 (2004), which is incorporated herein in its entirety by reference. PPARγ2 transactivates the expression of a number of target genes, including, for example, FABP4, LEP (NCBI Gene ID No 3952) and ADIPOQ (NCBI Gene ID No 9370). Thus, a PPARγ2 polypeptide useful in the methods described herein would also be expected to transactivate expression of one or more of these target genes or a reporter driven by transcription control elements from such gene(s). It is preferred that the transactivation domain be a naturally-occurring PPARγ2 transactivation domain or a conservative substitution variant thereof that retains the ability to transactivate PPARγ2 target gene expression. However, as noted below, it is contemplated that the transactivation domain can be a heterologous transactivation domain, including, potentially, a constitutively active transactivation domain that, when paired with a PPARγ2 DNA binding domain, permits ligand-independent transactivation of PPARγ2 responsive gene expression. A number of strong transactivation domains that would function in this capacity are known to those of skill in the art.

Naturally-occurring PPARγ2 polypeptides bind a variety of ligands that activate gene expression via the PPARγ2 ligand-activated transactivation domain AF-2. PPARγ2 polypeptides useful in the methods described herein include those that bind ligands bound by naturally-occurring PPARγ2 polypeptides. In addition, however, it is also contemplated that for the purposes of promoting the brown adipocyte differentiation program in stem, precursor or other cell types, in certain embodiments, the PPARγ2 polypeptide used can be a ligand-independent variant, i.e., a constitutively active PPARγ2 variant. Examples of PPARγ2 ligands include the thiazolidinedione and glitazar compounds described in the section “Agents that increase the level or activity of PPARγ2” herein. Such a variant is described, for example, by Li & Lazar (Mol. Endocrinol. 16: 1040-1048 (2002), which is incorporated herein by reference), who replaced the PPARγ2 transactivation domain with the constitutively active VP16 viral transactivation domain and demonstrated ligand-independent, specific activation of PPARγ2 -responsive genes. Thus, in the embodiment, the PPARγ2 polypeptide used in methods as described herein need not bind ligand(s) bound by naturally occurring PPARγ2 polypeptides, but due to the presence of a DNA binding domain that specifically binds PPARγ2 target genes, can specifically transactivate PPARγ2-responsive genes. Such a polypeptide will transactivate, for example, luciferase expression from the pPPREx3TK-luciferase reporter construct noted above, and/or expression from another PPARγ2-sensitive reporter as appropriate.

In addition to the pPPREx3TK-luciferase reporter construct discussed above herein, the activity of PPARγ2 can be determined, for example, by measuring the transcription of genes which are transactivated by a naturally occurring PPARγ2. Methods of measuring gene transcription are well known to those of skill in the art and include, by way of non-limiting example, quantitative RT-PCR or the use of reporter genes. Methods of designing primers for a gene of interest are known to those of ordinary skill in the art.

Also included among PPARγ2 polypeptides are conservative substitution variants of a mammalian PPARγ2 polypeptide that maintain PPPARγ2 activity as that term is described herein. PPARγ2 variants can be obtained by mutations of native PPARγ2 nucleotide sequences, for example. The domain structures as known in the art and as described herein provides guidance to one of ordinary skill in the art for the regions of PPARγ2 polypeptides that will tolerate modification yet likely retain PPARγ2 activity as the term is described herein. A “PPARγ2 variant,” as referred to herein, is a polypeptide substantially homologous to a native PPARγ2, but which has an amino acid sequence different from that of native PPARγ2 because of one or a limited number of deletions, insertions or substitutions. One of ordinary skill in the art will recognize that modifications can be introduced in a PPARγ2 sequence without destroying PPARγ2 activity. Such modified PPARγ2's can also be used in the methods described herein, e.g., if the modifications do not alter the DNA binding specificity or substantially adversely alter the transactivation activity(ies) of the protein relative to wild-type PPARγ2. In one embodiment, a variant will retain substantially normal ligand binding.

The variant amino acid or DNA sequence preferably is at least 90%, at least 91%, at least 92%, at least 93%, at least 94%, at least 95%, at least 96%, at least 97%, at least 98%, at least 99%, or more, identical to a native PPARγ2 sequence. The degree of homology (percent identity) between a native and a mutant sequence can be determined, for example, by comparing the two sequences using freely available computer programs commonly employed for this purpose on the world wide web.

Variants can comprise conservatively substituted sequences, meaning that one or more amino acid residues of a native PPARγ2 polypeptide are replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. PPARγ2 polypeptides comprising conservative amino acid substitutions can be tested in any one of the assays described herein to confirm that a desired activity of a PPARγ2 polypeptide is retained. By “retained” is meant that the activity is at least 50% of that of the wild-type polypeptide, preferably at least 60%, at least 70%, at least 80%, at least 90%, at least 100%, at least 150%, at least 200%, at least 300% or more, relative to wild-type.

Amino acids can be grouped according to similarities in the properties of their side chains in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H).

Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class.

Examples of conservative substitutions for use in the PPARγ2 variants described herein are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into IIe or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into IIe or into Leu.

Any cysteine residue not involved in maintaining the proper conformation of the PPARγ2 also can be substituted, generally with serine, to improve the oxidative stability of the molecule and prevent aberrant crosslinking. Conversely, cysteine bond(s) can be added to the PPARγ2 to improve its stability or facilitate oligomerization.

Alterations of the native amino acid sequence can be accomplished by any of a number of known recombinant DNA techniques are widely used in the art.

Agents that increase the level or activity of PPARγ2

An agent that can increase the level or activity of PPARγ2 can be, by way of non-limiting example, a nucleic acid, a polypeptide, or a small molecule. An agent that can increase the level or activity of PPARγ2 can be a PPARγ2 polypeptide as described herein above, or a nucleic acid encoding such a polypeptide.

The activity of PPARγ2 can be determined as described herein above.

The level of a PPARγ2 mRNA or protein can be determined by, for example, immunoassays (e.g., enzyme linked immunoabsorbant assay (ELISA), radioimmunoassay (RIA), immunoradiometric assay (IRMA)), Western blotting, PCR, or immunohistochemistry (including AQUA®). The level of PPARγ2 can be determined by, for example, quantitative RT-PCR as described in the Examples herein, or immunohistochemistry. Anti-PPARγ2 antibodies for immunohistochemistry are available commercially (e.g. Cat. #ab45278; Abcam; Cambridge, Mass.).

An agent that increases the level or activity of PPARγ2 can include an agent that inhibits proteasome degradation of PPARγ2 (see Kilroy et al. Obesity 2009 17:665-673, which is incorporated by reference herein in its entirety). Non-liming examples of agents that inhibit proteasome degradation include MG132 (Cat #C2211; Sigma-Aldrich; St. Louis, Mo.) and epoxomicin (Cat #E3652; Sigma-Aldrich; St. Louis, Mo.).

An agent that increases the activity of PPARγ2 can include ligands of a naturally-occurring PPARγ2 polypeptide. Examples of ligands useful in the methods described herein include, but are not limited to the thiazolidinedione and glitazar compounds described below herein. A ligand can be used to increase the activity of PPARγ2 in a cell which expresses a level of PPARγ2 polypeptide which is detectable using the methods described above herein, or a cell can be contacted with both a ligand of PPARγ2 and an agent which increases the level of PPARγ2.

A small molecule that can increase the level or activity of PPARγ2 can be, by way of non-limiting example, a thiazolidinedione or a glitazar and derivatives or salts thereof. As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

As used herein, a “thiazolidinedione” refers to a molecule comprising the moiety illustrated as Formula I.

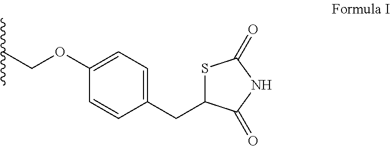 [SMILES: *COc1ccc(CC2SC(=O)NC2=O)cc1]

Thiazolidinediones are also referred to as glitazones by those of ordinary skill in the art. Thiazolidinediones can be specific activators of PPARγ, although certain individual thiazolidinediones (e.g. pioglitazone) can weakly activate other PPARs (See Yki-Jarvinen NEJM 2004 351:1106-1118 and Wilson et al. J Med Chem 1996 39:665-8, which are included by reference herein in their entirety). Thiazolidinediones contemplated for use in the methods described herein include, but are not limited to, rosiglitazone ((RS)-5-[4-(2-[methyl(pyridine-2-yl)amino]ethoxy)benzyl]thiazolidine-2,4-dione; Avandia; Formula II; Cayman Chemical; Ann Arbor, Mich.; Catalog No: 71740), pioglitazone ((RS)-5-(4-[2-(5-ethylpryidin-2-yl)ethyloxy]benzyl)thiazolidine-2,4-dione; Actos; Formula III; Cayman Chemical; Ann Arbor, Mich.; Catalog No: 71745); and troglitazone ((RS)-5-4-[(6-hydroxy-2,5,7,8-tetramethylchroman-2-yl) methoxy]benzyl)thiazolidine-2,4-dione; Rezulin; Formula IV; Cayman Chemical; Ann Arbor, Mich.; Catalog No: 71750); netoglitazone (5-[[6-[(2-fluorophenyl)methoxy]naphthalen-2-yl]methyl]-1,3-thiazolidine-2,4-dione; MCC-555; Formula V); rivoglitazone ((5-[[4-[2-(methyl-2-pyridinylamino)ethoxy]phenyl]methyl]-2,4-thiazolidinedione; Formula VI); ciglitazone (5-(4-[(1-methylcyclohexyl)methoxy]benzyl)-1,3-thiazolidine-2,4-dione; Formula VII; Cayman Chemical; Ann Arbor, Mich.; Catalog No: 71730); and balaglitzaone (5-[[4-[(3,4-dihydro-3-methyl-4-oxo-2-quinazolinyl) methoxy]phenyl]methyl]-2,4-Thiazolidinedione; Formula VII). Methods for making thiazolidienodiones and derivatives thereof are well known in the art. See, for example, U.S. Pat. Nos. 7,511,148; 5,441,971; 5,599,826; 6,787,551; 5,401,761; RE39384; 5,223,522; and 7,528,133, which are incorporated by reference herein in their entirety.

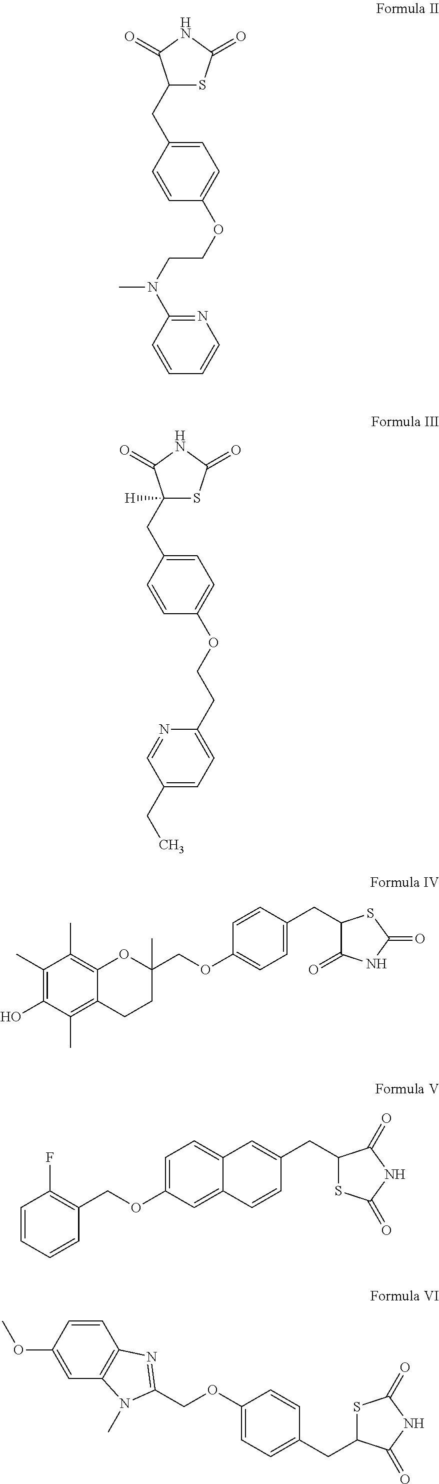 [SMILES: CN(CCOc1ccc(CC2SC(=O)NC2=O)cc1)c1ccccn1 ; CCc1ccc(CCOc2ccc(C[C@@H]3SC(=O)NC3=O)cc2)nc1 ; Cc1c(C)c2c(c(C)c1O)CCC(C)(COc1ccc(CC3SC(=O)NC3=O)cc1)O2 ; O=C1NC(=O)C(Cc2ccc3cc(OCc4ccccc4F)ccc3c2)S1 ; COc1ccc2nc(COc3ccc(CC4SC(=O)NC4=O)cc3)n(C)c2c1]

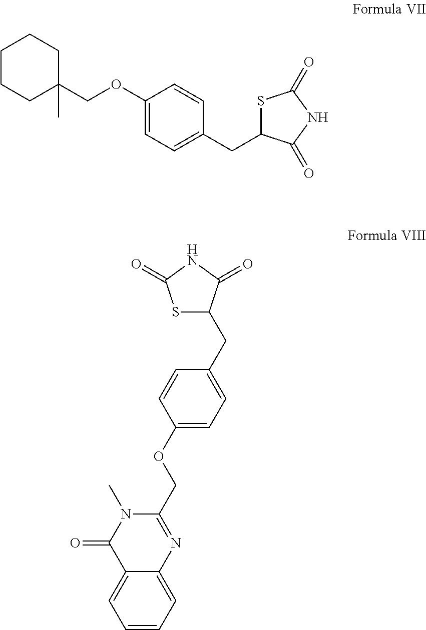 [SMILES: CC1(COc2ccc(CC3SC(=O)NC3=O)cc2)CCCCC1 ; Cn1c(COc2ccc(CC3SC(=O)NC3=O)cc2)nc2ccccc2c1=O]

In some embodiments, a population of cells to be differentiated to a brown adipocyte phenotype (e.g. stem, precursor or progenitor cells or other non-neuronal cells one wishes to differentiate) is contacted with a thiazolidinedione at a concentration of 0.005 μM to 50 μM. In some embodiments, a population of cells is contacted with a thiazolidinedione at a concentration of 0.05 μM to 5 μM. In some embodiments, a population of cells is contacted with a thiazolidinedione at a concentration of 0.1 μM to 1 μM.

As used herein, a “glitazar” refers to a molecule comprising a moiety as illustrated as Formula IX and/or Formula X, wherein R₁ is selected from the group consisting of C, N, and CN and R₂ is aromatic or hetero-aromatic.

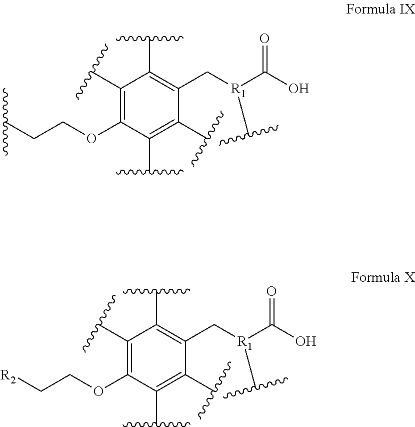

Glitazars are capable of increasing the activity of more than one PPAR isoform and/or genes. Examples of glitazars contemplated for use in the methods described herein include, but are not limited to, muraglitazar (N-[(4-methoxyphenoxy)carbonyl]-N-(4-[2-(5-methyl-2-phenyl-1,3-oxazol-4-yl)ethoxy]benzyl)glycine; Pargluva; Formula XI), tesaglitazar ((2S)-2-Ethoxy-3-[4-[2-(4-methylsulfonyloxyphenyl)ethoxy]phenyl]propanoic acid; Galida; Formula XII), aleglitazar ((2S)-2-methoxy-3-[4-(2-(5-methyl-2-phenyl-4-oxazolyl)ethoxy)-7-benzothiophenyl]propanoic acid; Formula XIII); farglitazar ((2S)-2-(2-benzoylanilino)-3-[4-[2-(5-methyl-2-phenyl-1, 3-oxazol-4-yl)ethoxy]phenyl]propanoic acid; Formula XIV); and ragaglitazar ((2S)-2-ethoxy-3-(4-(2-(10H-Phenoxazin-10-yl)ethoxy)phenyl)propanoic acid; Formula XV). Methods for making glitazars and derivatives thereof are well known in the art. See, for example, Patent Publication W09958510, WO 99/19313, WO 00/50414, WO 00/63191, WO 00/63192, WO 00/63193 and U.S. Pat. Nos. 6,414,002; B. Ljung et. al., /.Lipid Res., 2002, 43, 1855-1863; K. Yajima et. al., Am. J. Physiol. Endocrinol. Metab, 2003, 284: E966-E971; Murakami et al. Diabetes 47, 1841-1847 (1998); Devasfhale et. al., J Med Chem. Mar. 24, 2005;48(6):2248-50; Henke et al. Journal of Medicinal Chemistry 1998 41:5020-36; and Lohray et al. J Med Chem 2001 44:2675-8, which are incorporated by reference herein in their entirety.

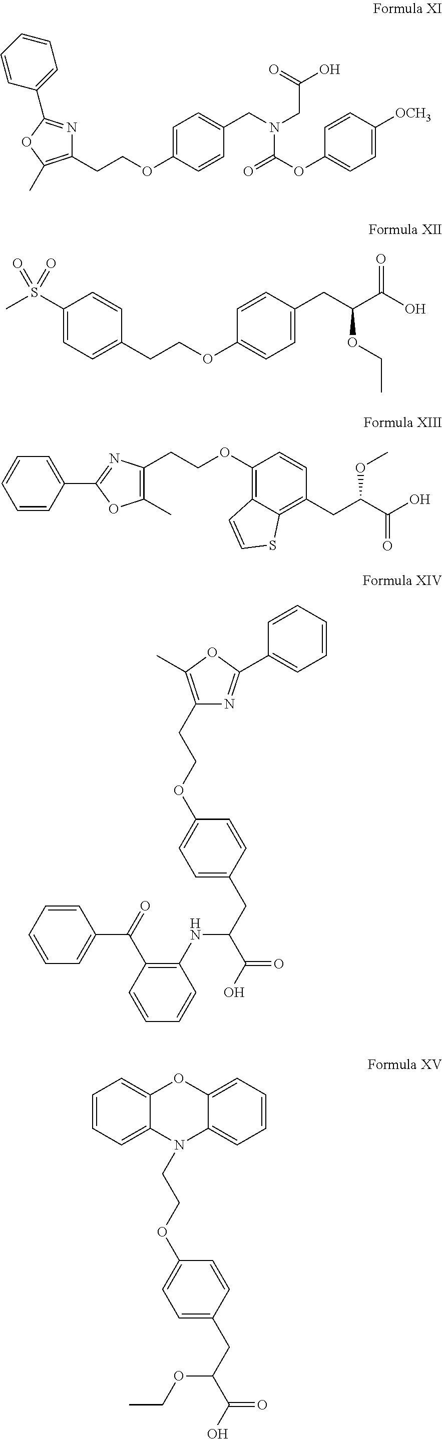 [SMILES: COc1ccc(OC(=O)N(CC(=O)O)Cc2ccc(OCCc3nc(-c4ccccc4)oc3C)cc2)cc1 ; CCO[C@@H](Cc1ccc(OCCc2ccc(S(C)(=O)=O)cc2)cc1)C(=O)O ; CO[C@@H](Cc1ccc(OCCc2nc(-c3ccccc3)oc2C)c2ccsc12)C(=O)O ; Cc1oc(-c2ccccc2)nc1CCOc1ccc(CC(Nc2ccccc2C(=O)c2ccccc2)C(=O)O)cc1 ; CCOC(Cc1ccc(OCCN2c3ccccc3Oc3ccccc32)cc1)C(=O)O]

Other examples of small molecules which can increase the level or activity of PPARγ2 include, but are not limited to, 15-deoxy-Δ^{12, 14} -prostaglandin J₂ (Formula XVI) (Cayman Chemical; Ann Arbor, Mich.; Catalog No: 18570); AR-H039242 (Aztrazeneca), GW-409544 (Glaxo-Wellcome), nTZDpa (5-Chloro.-1-[(4-chlorophenyl)methyl]-3-(phenylthio)-1H-indole-2-carboxylic acid; Cat #2150 Tocris Bioscience; Mo.); BVT-142, CLX0940, GW-1536, GW-1929 (Cat. # 1664 Tocris Bioscience; Ellisville, Mo.), GW-2433, KRP-297 (Kyorin Merck; 5-[(2,4-Dioxo thiazolidinyl)methyl]methoxy-N-([4(trifluoromethyl)phenyl] methyljbenzamide), L-796449, LR-90, MK-0767 (Merck/Kyorin/Banyu), the angiotensin receptor blocker telismartan (2-{[4-methyl-6-(1-methyl-1H-1,3-benzodiazol-2-yl)-2-propyl-1H-1,3-benzodiazol-1-yl] methyl}phenyl)benzoic acid) and SB 219994 and those disclosed in W099/16758, W099/19313, WO99/20614, WO99/38850, WO00/23415, WO00/23417, WO00/23445, WO00/50414, WO01/00579, WO01/79150, WO02/062799, WO03/004458, WO03/016265, WO03/018010, WO03/033481, WO03/033450, WO03/033453, WO03/043985, WO031053976, U.S. application Ser. No. 09/664,598, filed Sep. 18,2000, Murakami et al. Diabetes 47, 1841-1847 (1998).

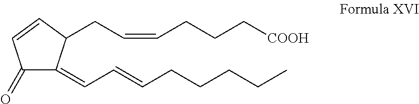 [SMILES: CCCCC/C=C/C=C1/C(=O)C=CC1C/C=C\CCCC(=O)O]

In some embodiments, an agent that can increase the level or activity of PPARγ2 can be a nucleic acid encoding a PPARγ2 polypeptide as described herein above. In some embodiments, the nucleic acid encoding a PPARγ2 polypeptide can be the nucleic acid of SEQ ID NO: 01, a homologous nucleic acid with a sequence identity of, for example, at least at least 90%, 95%, 99%, or even 100%, over a region spanning 50, 100,150,200,250,300,350,400, 450, 500, or even more nucleotides. One of ordinary skill in the art will also recognize that modifications can be introduced in a PPARγ2 sequence without destroying PPARγ2 activity. Such modified PPARγ2′s can also be used in the present invention, e.g., if the modifications do not alter the DNA binding site conformation to the extent that the modified PPARγ2 lacks substantially normal DNA binding. Any PPARγ2 variant, homologue, or mutant can be used in the present invention if it retains PPARγ2 activity as discussed herein above. A homologous polypeptide can be a peptide with a sequence identity of, for example, at least 90%, 95%, 99%, or even 100%, over a region spanning 50, 100,150, 200,250,300,350,400, 450, 500, or even more amino acids as compared to a PPARγ2 polypeptide as described above herein.

The agent can comprise a vector. Many vectors useful for transferring exogenous genes into target mammalian cells are available. The vectors can be episomal, e.g., plasmids, virus derived vectors such cytomegalovirus, adenovirus, etc., or can be integrated into the target cell genome, through homologous recombination or random integration, e.g., retrovirus derived vectors such MMLV, HIV-1, ALV, etc. Many viral vectors are known in the art and can be used as carriers of a nucleic acid modulatory compound into the cell. For example, constructs containing the nucleic acid encoding a polypeptide can be integrated and packaged into non-replicating, defective viral genomes like Adenovirus, Adeno-associated virus (AAV), or Herpes simplex virus (HSV) or others, including retroviral and lentiviral vectors, for infection or transduction into cells. Alternatively, the construct can be incorporated into vectors capable of episomal replication, e.g. EPV and EBV vectors. The nucleic acid incorporated into the vector can be operatively linked to an expression control sequence such that the expression control sequence controls and regulates the transcription and translation of that polynucleotide sequence.

### C/EBPβ Polypeptides

C/EBPβ is a CCAAT/Enhancer-binding transcription factor. Agents that increase the activity or expression of a C/EBPβ polypeptide are used in the methods described herein, in conjunction with agents that increase the activity or expression of a PPARγ2 polypeptide, to promote the differentiation of mammalian cells to a brown fat phenotype. In certain embodiments, C/EBPβ polypeptides themselves can be administered, e.g., either directly, or more often, via expression from a nucleic acid construct, in order to increase the expression and activity of C/EBPβ in the cell targeted for differentiation. In other embodiments, for example, chemical agents and/or small molecules that increase either or both of the expression or activity of a C/EBPβ polypeptide can be used. C/EBPβ is also referred to variously as “C/EBPB,” “C/EBPbeta” or “CEBPB.” C/EBPβ is also known in the art as C/EBP2, LAP, TCF5, CRP2, 35 NFIL6DBP, NF-M, AGP/EBP and Apc/EPB.

At a basic level, a C/EBPβ polypeptide is a polypeptide derived from or similar to C/EBPβ that retains the ability to direct or promote the differentiation of a stem or progenitor cell to a brown adipocyte phenotype in conjunction with an agent that increases the activity or expression of a PPARγ2 polypeptide. Discussed below are the structural determinants identified for C/EBPβ polypeptides, as well as functions, including determinants of target gene binding and transactivation by C/EBPβ polypeptides useful according to the methods described herein.

There are at least three naturally-occurring isoforms of C/EBPβ; a 38 kDA form known as LAP* (SEQ ID NO: 04), a 35-kDa form (corresponding to amino acids 24-345 of SEQ ID NO: 04), known as LAP and a 20-kDa form known as LIP (corresponding to amino acids 200-345 of SEQ ID NO: 04), which result from alterative start sites and thus vary from each other in the N-terminus. It is notable that LIP is a dominantly interfering isoform, as opposed to the transcriptional activators LAP and LAP*, indicating that amino acids 1-200 are necessary for transactivation. Exemplary reference sequences are the human C/EBPβ nRNA (SEQ ID NO: 03; NCBI Reference Sequence: NM_{—}005194) and protein sequences (SEQ ID NO: 04; NCBI Reference Sequence: NP_{—}005185). One of ordinary skill in the art will recognize that sequence differences will exist due to allelic variation, and will also recognize that other animals, particularly other mammals, have corresponding C/EBPβs, which have been identified or can be readily identified using sequence alignment and confirmation of activity.

Naturally-occurring C/EBPβ polypeptides generally comprise modular structural domains including (a) a transactivation domain (TAD) comprising four conserved regions (CR1-CR4) (corresponding to amino acids 1-113 of SEQ ID NO:4), (b) a composite regulatory domain (RD) comprising 3 conserved regions (CR5-CR7) and which controls TAD functioning (corresponding to amino acids 114-192 of SEQ ID NO. 4) and (c) a highly conserved C-terminal basic-leucine zipper (bZIP) domain (corresponding to at least amino acids 269-334 of SEQ ID NO: 04) (Williams et al. EMBO Journal 1995 14:3170-3183; Leutz et al. Transcription 2011 2:1, 3-8; Kownez-Leutz et al. EMBO Journal 2010 29:1105-1115; which are incorporated by reference herein in their entirety). C/EBPβ polypeptides useful according to the methods described herein include naturally-occurring mammalian C/EBPβ polypeptides including, but not limited to human C/EBPβ having the amino acid sequence at GenBank accession No. NP_{—}005185 (SEQ ID NO: 04), murine C/EBPβ having the amino acid sequence at GenBank accession No. NP_{—}034013 (SEQ ID NO: 12), is well as rat C/EBPβ having the amino acid sequence at GenBank accession No. NP_{—}077039 (SEQ ID NO: 13).

At a minimum, a “C/EBPβ polypeptide” as the term is used herein, can bind the regulatory region of a C/EBPβ-responsive target gene (i.e., a target gene transactivated by a naturally-occurring C/EBPβ polypeptide) and transactivate (that is, up-regulate) the expression of that gene. Thus, “C/EBPβ activity” refers to the sequence-specific transactivation of a gene that is transactivated by a naturally-occurring, wild-type C/EBPβ polypeptide.

C/EBPβ polypeptides useful in methods as described herein include a bZIP domain that binds to one or more DNA sequences bound by wild-type, naturally-occurring mammalian C/EBPβ polypeptides. Non-limiting examples of a consensus sequence for C/EBPβ are T[TG]NNGNAA[TG] (SEQ ID NO: 18) and TCGCCTAGCATTTCATCACACGT (SEQ ID NO: 19). (See Taniguchi et al. FEBS Letters 2005 579:5785-5790 and Karaya et al. Nucleic Acids Research 2005 33:1924-1934, both of which are incorporated by reference herein in their entirety. It is preferred that the C/EBPβ polypeptide includes a basic leucine zipper DNA binding domain that corresponds, for example, to the DNA binding domain of C/EBPβ. At a minimum, a C/EBPβ polypeptide as described herein bind to the C/EBPβ-sensitive promoter of the peroxisome proliferator-activated receptor gamma, coactivator 1 alpha (PGC1A) (NCBI Gene ID No 10891) gene and/or the uncoupling protein 1 (UCP1) (NCBI Gene ID No 7350) gene. Basic leucine zipper DNA binding domains are well known in the art, and the structural determinants within them that provide for specificity for a given DNA sequence are apparent to those of skill in the art.

C/EBPβ polypeptides useful in methods as described herein include a transactivation domain (or domains) that, at a minimum, transactivates expression from a C/EBPβ sensitive reporter gene construct, such as the C/EBPβ-sensitive reporter gene construct [−81;+103]-pCATBASIC described by Thomas et al. Eur. J. Biochem. 2000 267:6798-6809, which is incorporated herein in its entirety by reference. C/EBPβ transactivates the expression of a number of target genes, including, for example, UCP1 and PGC1A. Thus, a C/EBPβ polypeptide useful in the methods described herein would also be expected to transactivate expression of one or more of these target genes or a reporter driven by transcription control elements from such gene(s). It is preferred that the transactivation domain be a naturally-occurring C/EBPβ transactivation domain or a conservative substitution variant thereof that retains the ability to transactivate C/EBPβ target gene expression. However, as noted below, it is contemplated that the transactivation domain can be a heterologous transactivation domain, including, potentially, a constitutively active transactivation domain that, when paired with a C/EBPβ DNA binding domain, permits constitutive transactivation of C/EBPβ responsive gene expression, for example, as described in Williams et al. A number of strong transactivation domains that would function in this capacity are known to those of skill in the art.

Naturally-occurring C/EBPβ polypeptides are subject to methylation and phosphorylation which influence the activity of C/EBPβ. By way of non-limiting example, a lack of methylation of the R3 residue of LAP* is known to correlate with increased expression of adipogenic genes (Kowenz-Leutz 1994 Genes & Development 8:2781-2791) while phosphorylation of the Thr235 and Ser105 residues increases the transactivation mediated by C/EBPβ (Williams et al 1995). C/EBPβ polypeptides useful in the methods described herein include those that are subject to the same phosphorylationmethylation patterns as naturally-occurring C/EBPβ polypeptides; or which are disposed to activating post-translational modifications (e.g. C/EBPβ polypeptides which are phosphorylated at Thr235 and Ser105 prior to administration, or which contain mutations abolishing methylation at R3). In addition, it is also contemplated that for the purposes of promoting the brown adipocyte differentiation program in stem, precursor or other cell types, in certain embodiments, the C/EBPβ polypeptide used can be a constitutively active C/EBPβ variant. Such a polypeptide will transactivate, for example, luciferase expression from the [−81;+103]-pCATBASIC reporter construct noted above, and/or expression from another C/EBPβ-sensitive reporter as appropriate.

In addition to the [−81;+103]-pCATBASIC reporter construct discussed above herein, the activity of C/EBPβ can be determined, for example, by measuring the transcription of genes which are transactivated by a naturally occurring C/EBPβ. Methods of measuring gene transcription are well known to those of skill in the art and include, by way of non-limiting example, quantitative RT-PCR or the use of reporter genes. Methods of designing primers for a gene of interest are known to those of ordinary skill in the art.

Also included are conservative substitution variants of a mammalian C/EBPβ polypeptide that maintain C/EBPβ activity as that term is described herein. C/EBPβ variants can be obtained by mutations of native PPARγ2 nucleotide sequences, for example. A “C/EBPβ variant,” as referred to herein, is a polypeptide substantially homologous to a native C/EBPβ, but which has an amino acid sequence different from that of native C/EBPβ because of one or a plurality of deletions, insertions or substitutions. One of ordinary skill in the art will recognize that modifications can be introduced in a C/EBPβ sequence without destroying C/EBPβ activity. Such modified C/EBPβ′s can also be used in the present invention, e.g., if the modifications do not alter the binding site conformation to the extent that the modified C/EBPβ lacks substantially normal ligand binding.

The variant amino acid or DNA sequence preferably is at least 90%, at least 91%, at least 92%, at least 93%, at least 94%, at least 95%, at least 96%, at least 97%, at least 98%, at least 99%, or more, identical to a native C/EBPβ sequence. The degree of homology (percent identity) between a native and a mutant sequence can be determined, for example, by comparing the two sequences using freely available computer programs commonly employed for this purpose on the world wide web.

Variants can comprise conservatively substituted sequences, meaning that one or more amino acid residues of a native C/EBPβ polypeptide are replaced by different residues, and that the conservatively substituted C/EBPβ polypeptide retains a desired biological activity, i.e., C/EBPβ, that is essentially equivalent to that of the native C/EBPβ polypeptide. Examples of conservative substitutions include substitution of amino acids that do not alter the secondary and/or tertiary structure of C/EBPβ. conservative substitutions and the methods of making substitutions in a polypeptide are described above herein.

Agents that increase the level or activity of CEBPβ

An agent that can increase the level or activity of C/EBPβ can be, by way of non-limiting example, a nucleic acid, a polypeptide, or a small molecule. An agent that can increase the level or activity of C/EBPβ can be a C/EBPβ polypeptide as described herein above.

The activity of C/EBPβ can be determined as described herein above.

The level of a C/EBPβ mRNA or protein can be determined by, for example, immunoassays (e.g., enzyme linked immunoabsorbant assay (ELISA), radioimmunoassay (RIA), immunoradiometric assay (IRMA)), Western blotting, PCR, or immunohistochemistry (including AQUA®). The level of C/EBPβ can be determined by, for example, quantitative RT-PCR as described in the Examples herein, or immunohistochemistry. Anti-C/EBPβ antibodies for immunohistochemistry are available commercially (e.g. Cat. #ab32358; Abcam; Cambridge, Mass.).

In some embodiments, an agent that can increase the level or activity of C/EBPβ can be a nucleic acid encoding a C/EBPβ polypeptide as described above. In some embodiments, the nucleic acid encoding a C/EBPβ polypeptide can be the nucleic acid of SEQ ID NO: 01, a homologous nucleic acid with a sequence identity of, for example, at least at least 90%, 95%, 99%, or even 100%, over a region spanning 50, 100,150, 200,250,300,350,400, 450, 500, or even more nucleotides. One of ordinary skill in the art will also recognize that modifications can be introduced in a C/EBPβ sequence without destroying C/EBPβ activity. Such modified C/EBPβ′s can also be used in the present invention, e.g., if the modifications do not alter the DNA binding site conformation to the extent that the modified C/EBPβ lacks substantially normal DNA binding. Any C/EBPβ variant, homologue, or mutant can be used in the present invention if it retains C/EBPβ activity as discussed above herein. A homologous polypeptide can be a peptide with a sequence identity of, for example, at least 90%, 95%, 99%, or even 100%, over a region spanning 50, 100, 150, 200, 250,300,350,400, 450, 500, or even more amino acids as compared to a C/EBPβ polypeptide as described above herein.

The agent can comprise a vector. Many vectors useful for transferring exogenous genes into target mammalian cells are available. The vectors can be episomal, e.g., plasmids, virus derived vectors such cytomegalovirus, adenovirus, etc., or can be integrated into the target cell genome, through homologous recombination or random integration, e.g., retrovirus derived vectors such MMLV, HIV-1, ALV, etc. Many viral vectors are known in the art and can be used as carriers of a nucleic acid modulatory compound into the cell. For example, constructs containing the nucleic acid encoding a polypeptide can be integrated and packaged into non-replicating, defective viral genomes like Adenovirus, Adeno-associated virus (AAV), or Herpes simplex virus (HSV) or others, including retroviral and lentiviral vectors, for infection or transduction into cells. Alternatively, the construct can be incorporated into vectors capable of episomal replication, e.g. EPV and EBV vectors. The nucleic acid incorporated into the vector can be operatively linked to an expression control sequence such that the expression control sequence controls and regulates the transcription and translation of that polynucleotide sequence.

### Viral Delivery of Nucleic Acid Agents

Agents comprising a nucleic acid, as described herein, can be delivered to a cell using a viral vector. Retroviruses, such as lentiviruses, provide a convenient platform for delivery of nucleic acid sequences encoding an agent of interest. A selected nucleic acid sequence can be inserted into a vector and packaged in retroviral particles using techniques known in the art. The recombinant virus can then be isolated and delivered to cells, e.g. in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Calster Academic Press (ISBN:978-1-90455-55-4); and Hu and Pathak Pharmacological Reviews 2000 52:493-512; which are incorporated by reference herein in their entirety.

### Adenoviral Delivery

In some embodiments, a nucleotide sequence encoding an agent of interest is inserted into an adenovirus-based expression vector. Unlike retroviruses, which integrate into the host genome, adenoviruses persist extrachromosomally thus minimizing the risks associated with insertional mutagenesis (Haj-Ahmad and Graham (1986) J. Virol. 57:267-74; Bett et al. (1993) J. Virol. 67:5911-21; Mittereder et al. (1994) Human Gene Therapy 5:717-29; Seth et al. (1994) J. Virol. 68:933-40; Barr et al. (1994) Gene Therapy 1:51-58; Berkner, K. L. (1988) BioTechniques 6:616-29; and Rich et al. (1993) Human Gene Therapy 4:461-76). Adenoviral vectors have several advantages in gene therapy. They infect a wide variety of cells, have a broad host-range, exhibit high efficiencies of infectivity, direct expression of heterologous sequences at high levels, and achieve long-term expression of those sequences in vivo. The virus is fully infective as a cell-free virion so injection of producer cell lines is not necessary. With regard to safety, adenovirus is not associated with severe human pathology, and the recombinant vectors derived from the virus can be rendered replication defective by deletions in the early-region 1 (“E1”) of the viral genome. Adenovirus can also be produced in large quantities with relative ease. For all these reasons vectors derived from human adenoviruses, in which at least the E1 region has been deleted and replaced by a gene of interest, have been used extensively for gene therapy experiments in the pre-clinical and clinical phase.

Adenoviral vectors for use with the compositions and methods described herein can be derived from any of the various adenoviral serotypes, including, without limitation, any of the over 40 serotype strains of adenovirus, such as serotypes 2, 5, 12, 40, and 41. The adenoviral vectors of used in the methods described herein are generally replication-deficient and contain the sequence of interest under the control of a suitable promoter. For example, U.S. Pat. No. 6,048,551, incorporated herein by reference in its entirety, describes replication-deficient adenoviral vectors that include a human gene under the control of the Rous Sarcoma Virus (RSV) promoter. Other recombinant adenoviruses of various serotypes, and comprising different promoter systems, can be created by those skilled in the art. See, e.g., U.S. Pat. No. 6,306,652, incorporated herein by reference in its entirety.

Other useful adenovirus-based vectors for delivery of nucleic acid sequence encoding a PPARγ2 and/or C/EBPβ polypeptide include, but are not limited to: “minimal” adenovirus vectors as described in U.S. Pat. No. 6,306,652, which retain at least a portion of the viral genome required for encapsidation (the encapsidation signal), as well as at least one copy of at least a functional part or a derivative of the ITR; and the “gutless” (helper-dependent) adenovirus in which the vast majority of the viral genome has been removed and which produce essentially no viral proteins, such vectors can permit gene expression to persist for over a year after a single administration (Wu et al. (2001) Anesthes. 94:1119-32; Parks (2000) Clin. Genet. 58:1-11; Tsai et al. (2000) Curr. Opin. Mol. Ther. 2:515-23).

### Adeno Associated Virus (AAV) Delivery

In some embodiments, a nucleotide sequence encoding a PPARγ2 and/or C/EBPβ polypeptide is inserted into an adeno-associated virus-based expression vector. AAV is a parvovirus which belongs to the genus Dependovirus and has several features not found in other viruses. AAV can infect a wide range of host cells, including non-dividing cells. AAV can infect cells from different species. AAV has not been associated with any human or animal disease and does not appear to alter the biological properties of the host cell upon integration. Indeed, it is estimated that 80-85% of the human population has been exposed to the virus. Finally, AAV is stable at a wide range of physical and chemical conditions, facilitating production, storage and transportation.

AAV is a helper-dependent virus; that is, it requires co-infection with a helper virus (e.g., adenovirus, herpesvirus or vaccinia) in order to form AAV virions in the wild. In the absence of co-infection with a helper virus, AAV establishes a latent state in which the viral genome inserts into a host cell chromosome, but infectious virions are not produced. Subsequent infection by a helper virus rescues the integrated genome, allowing it to replicate and package its genome into infectious AAV virions. While AAV can infect cells from different species, the helper virus must be of the same species as the host cell. Thus, for example, human AAV will replicate in canine cells co-infected with a canine adenovirus.

Adeno-associated virus (AAV) has been used with success in gene therapy. AAV has been engineered to deliver genes of interest by deleting the internal nonrepeating portion of the AAV genome (i.e., the rep and cap genes) and inserting a heterologous sequence (in this case, the sequence encoding the agent) between the ITRs. The heterologous sequence is typically functionally linked to a heterologous promoter (constitutive, cell-specific, or inducible) capable of driving expression in the patient's target cells under appropriate conditions.

Recombinant AAV virions comprising a nucleic acid sequence encoding an agent of interest can be produced using a variety of art-recognized techniques, as described in U.S. Pat. Nos. 5,139,941; 5,622,856; 5,139,941; 6,001,650; and 6,004,797, the contents of each of which are incorporated by reference herein in their entireties. Vectors and cell lines necessary for preparing helper virus-free rAAV stocks are commercially available as the AAV Helper-Free System (Catalog No. 240071) (Agilent Technologies, Santa Clara, Calif.).

### Other Viral Vectors for Delivery

Additional viral vectors useful for delivering nucleic acid molecules encoding a PPARγ2 or C/EBPβ polypeptide include those derived from the pox family of viruses, including vaccinia virus and avian poxvirus. Alternatively, avipoxviruses, such as the fowlpox and canarypox viruses, can be used to deliver the genes. The use of avipox vectors in cells of human and other mammalian species is advantageous with regard to safety because members of the avipox genus can only productively replicate in susceptible avian species. Methods for producing recombinant avipoxviruses are known in the art and employ genetic recombination, see, e.g., WO 91/12882; WO 89/03429; and WO 92/03545.

Molecular conjugate vectors, such as the adenovirus chimeric vectors, can also be used for delivery of sequence encoding a PPARγ2 or C/EBPβ polypeptide (Michael et al. (1993) J. Biol. Chem. 268:6866-69 and Wagner et al. (1992) Proc. Natl. Acad. Sci. USA 89:6099-6103). Members of the Alphavirus genus, for example the Sindbis and Semliki Forest viruses, can also be used as viral vectors for delivering a nucleic acid sequence (See, e.g., Dubensky et al. (1996) J. Virol. 70:508-19; WO 95/07995; WO 96/17072).

In some embodiments, multiple agents that increase the level or activity of either C/EBPβ or PPARγ2 are used. By way of non-limiting example, a nucleic acid encoding a PPARγ2 can be used to increase the level of PPARγ2 in a cell and, in the same cell, rosiglitazone can be used to increase the activity of both the endogenous and exogenous PPARγ2.

### Differentiation

Provided herein are methods for promoting the differentiation of stem or progenitor cells into brown adipocytes in vitro and/or ex vivo. In one aspect the method comprises (a) providing a population of stem or progenitor cells; (b) contacting the population of stem or progenitor cells with at least one agent that increases the level or activity of PPARγ2 and C/EBPβ and (c) culturing the cells under conditions favorable for differentiation into brown adipocytes. The method does not require or comprise contacting the cells with an agent that increases the level or activity of PRDM16.

In one aspect the method comprises (a) differentiating pluripotent stem cells into mesenchymal stem cells; (b) contacting the mesenchymal stem cells with at least one agent that increases the level or activity of PPARγ2 and C/EBPβ and (c) culturing the cells under conditions favorable for the differentiation into brown adipocytes. The method does not require or comprise contacting the cells with an agent that increases the level or activity of PRDM16.

As used herein, “culturing the cells under conditions favorable for differentiation” refers to culturing the cells in media and environmental conditions which will allow the differentiation of brown adipocytes. By way of non-limiting example, appropriate conditions include medium containing DMEM, 7.5% knockout serum replacement (KOSR; Invitrogen), 7.5% human plasmanate, 0.5% nonessential amino acids, 1% penicillin/streptomycin, 0.1 μM dexamethasone and 10 μg/ml insulin (Sigma) at 37° C.

Further non-limiting examples include; (1) media containing steroids, a cyclic AMP inducer, and fatty acids and (2) DMEM/F-12 with 3% FBS, 3 μM biotin, 17 μM pantothenate, 1 μM bovine insulin, 1 μM dexamethasone, 0.5 mM isobutylmethylxanthine (IBMX), and 100 U penicillin/100 μg streptomycin/0.25 μg fungizone (described further in U.S. Pat. No. 6,322,784; 7,001,746; U.S. Patent Publication Nos. 2005/0158706; and Mitchella et al. Stem cells 2006 24:376-385; Zuk et al. Tissue Eng 2001 7:211-228; and Gimble et al., Cytotherapy 2003 5:362-9, which are incorporated by reference herein in their entirety);

Once differentiated, brown adipocytes can be maintained in an adipocyte maintenance medium. Non-limiting examples include (1) DMEM/F-12 with 3% FBS, 3 μM biotin, 17 μM pantothenate, 1 μM bovine insulin, 1 μM dexamethasone, and 100 U penicillin/100 μg streptomycin/0.25 μg fungizone or (2) DMEM, 7.5% knockout serum replacement (KOSR; Invitrogen), 7.5% human plasmanate, 0.5% nonessential amino acids, 1% penicillin/streptomycin, 0.1 μM dexamethasone and 10 μg/ml insulin (Sigma) at 37° C.

As used herein, the term “contacting a cell with an agent which increases the level (or activity) of PRDM16” refers to the forced expression of PRDM16 in a cell by introduction of a nucleic acid construct (e.g. virus, plasmid, etc.) that encodes PRDM16. It is preferred that such an agent not be an agent that indirectly induces PRDM16 expression.

As used herein, “PRDM16 polypeptide” refers to a zinc finger transcription factor that promotes the differentiation of cells towards a brown fat fate and prevents differentiation towards a muscle cell fate. PRDM16 transactivates expression of genes including UCP1, CIDEA, COX8B, ELOVL3, CMT1A, NDUFA11, NDUFA13, CYC1, DIO2, LHX8, COX8A, and CYFIP2.

Exemplary reference sequences are the human PRDM16 mRNA (SEQ ID NOs: 05-06; NCBI Reference Sequences: NM_{—}022114, NM_{—}199454) and protein sequences (SEQ ID NOs: 07-08; NCBI Reference Sequences: NP_{—}071397, NP_{—}955533).

As used herein “the rate of differentiation” refers to the proportion at which stem or progenitor cells differentiate into brown adipocytes. Thus, in a population in which the rate of differentiation to brown adipocytes is 80%, 80% of the stem, progenitor or precursor cells in a population differentiate to brown adipocytes.

### Screening

Described herein are methods for screening for agents that increase the activity or development of brown adipocytes. The methods comprise contacting cells with a candidate agent and assaying for brown adipocyte differentiation.

It is contemplated that one can screen for agents, e.g. small molecules that substitute for either of PPARγ2 or C/EBPβ to drive the differentiation of brown adipocytes. In this aspect, a cell which is to be differentiated to a brown adipocyte (e.g. a stem, progenitor, or precursor cell or a fibroblast) is contacted with an agent that increases the level or activity of either PPARγ2 or C/EBPβ and a candidate agent, and cells are cultured under conditions to permit differentiation. The differentiation of cells with a brown adipocyte phenotype indicates that the agent can substitute for the omitted PPARγ2 or C/EBPβ polypeptide.

In one aspect, the invention is directed to a method of screening for agents that increase the development of brown adipocytes. The method comprises (a) contacting stem cells or progenitor cells with at least one agent that increases the level or activity of PPARγ2 and C/EBPβ (b) contacting the cells with an additional candidate agent; and (c) culturing the cells under conditions favorable for differentiation into brown adipocytes. A candidate agent is identified as an agent that increases the development of brown adipocytes if the rate of proliferation or rate of differentiation of brown adipocytes is higher in the presence of the candidate agent.

In another aspect, brown adipocytes generated according to the methods described herein can be cultured in the presence of a candidate agent to identify agents that, for example, increase or modulate the proliferation or metabolic rate of the brown adipocytes. Combining this approach with an appropriate assay for the desired activity provides a powerful method to identify, for example, an agent to promote the proliferation or metabolic activity of brown adipocytes in vivo.

In one aspect, the invention is directed to a method for screening for agents that increase the activity of brown adipocytes. The method comprises (a) contacting stem cells or progenitor cells with at least one agent that increases the level or activity of PPARγ2 and C/EBPβ (b) culturing the cells under conditions favorable for differentiation into brown adipocytes; and (c) contacting the brown adipocytes with a candidate agent. A candidate agent is identified as an agent that increases the activity of brown adipocytes if a measure of brown adipocyte activity is higher in the presence of the candidate agent.

As used herein, a “candidate agent” refers to any entity which is normally not present or not present at the levels being administered to a cell, tissue or subject. A candidate agent can be selected from a group comprising: chemicals; small organic or inorganic molecules; nucleic acid sequences; nucleic acid analogues; proteins; peptides; aptamers; peptidomimetic, peptide derivative, peptide analogs, antibodies; intrabodies; biological macromolecules, extracts made from biological materials such as bacteria, plants, fungi, or animal cells or tissues; naturally occurring or synthetic compositions or functional fragments thereof. In some embodiments, the candidate agent is any chemical, entity or moiety, including without limitation synthetic and naturally-occurring non-proteinaceous entities. In certain embodiments the candidate agent is a small molecule having a chemical moiety. For example, chemical moieties include unsubstituted or substituted alkyl, aromatic, or heterocyclyl moieties including macrolides, leptomycins and related natural products or analogues thereof. Candidate agents can be known to have a desired activity and/or property, or can be selected from a library of diverse compounds.

Candidate agents can be screened for their ability to increase the development, proliferation and/or activity of brown adipocytes in vitro. In one embodiment, candidate agents are screened using the assays for brown adipocyte development and/or activity described below herein.

Generally, compounds can be tested at any concentration that can modulate cellular function, gene expression or protein activity relative to a control over an appropriate time period. In some embodiments, compounds are tested at concentration in the range of about 0.1 nM to about 1000 mM. In one embodiment, the compound is tested in the range of about 0.1 μM to about 20 μM, about 0.1 μM to about 10 μM, or about 0.1 μM to about 5 μM.

Depending upon the particular embodiment being practiced, the candidate or test compounds can be provided free in solution, or can be attached to a carrier, or a solid support, e.g., beads. A number of suitable solid supports can be employed for immobilization of the test compounds. Examples of suitable solid supports include agarose, cellulose, dextran (commercially available as, e.g., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, test compounds can be screened individually, or in groups. Group screening is particularly useful where hit rates for effective test compounds are expected to be low such that one would not expect more than one positive result for a given group.

Methods for developing small molecule, polymeric and genome based libraries are described, for example, in Ding, et al. J Am. Chem. Soc. 124: 1594-1596 (2002) and Lynn, et al., J. Am. Chem. Soc. 123: 8155-8156 (2001). Commercially available compound libraries can be obtained from, e.g., ArQule (Woburn, Mass.), Invitrogen (Carlsbad, Calif.), Ryan Scientific (Mt. Pleasant, S.C.), and Enzo Life Sciences (Farmingdale, N.Y.). These libraries can be screened for the ability of members to increase brown adipocyte activity and/or development using e.g. methods described herein.

The candidate agents can be naturally occurring proteins or their fragments. Such candidate agents can be obtained from a natural source, e.g., a cell or tissue lysate. Libraries of polypeptide agents can also be prepared, e.g., from a cDNA library commercially available or generated with routine methods. The candidate agents can also be peptides, e.g., peptides of from about 5 to about 30 amino acids, with from about 5 to about 20 amino acids being preferred and from about 7 to about 15 being particularly preferred. The peptides can be digests of naturally occurring proteins, random peptides, or “biased” random peptides. In some methods, the candidate agents are polypeptides or proteins. Peptide libraries, e.g. combinatorial libraries of peptides or other compounds can be fully randomized, with no sequence preferences or constants at any position. Alternatively, the library can be biased, i.e., some positions within the sequence are either field constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The candidate agents can also be nucleic acids. Nucleic acid candidate agents can be naturally occurring nucleic acids, random nucleic acids, or “biased” random nucleic acids. For example, digests of prokaryotic or eukaryotic genomes can be similarly used as described above for proteins.

In some embodiments, the candidate agent that is screened and identified to increase development, proliferation and/or activity of brown adipocytes according to the methods described herein, can increase development, proliferation and/or activity of brown adipocytes by at least 5%, preferably at least 10%, 20%, 30%, 40%, 50%, 50%, 70%, 80%, 90%, 1-fold, 1.1-fold, 1.5-fold, 2-fold, 3-fold, 4-fold, 5-fold, 10-fold, 50-fold, 100-fold or more higher relative to an untreated control.

The candidate agent can function directly in the form in which it is administered. Alternatively, the candidate agent can be modified or utilized intracellularly to produce a form that modulates the desired activity, e.g. introduction of a nucleic acid sequence into a cell and its transcription resulting in the production of an inhibitor or activator of gene expression or protein activity within the cell.

### Brown Adipocyte Activity and Development

The activity of brown adipocytes can be measured using any of several methods well known to those of skill in the art. By way of non-limiting example, brown adipocyte activity can be measured by measuring the generation of heat, the rate of growth, the rate of proliferation, the number of mitochondria and their activity, glycerol release and the expression of brown adipocyte marker genes (e.g. UCP1, ELOVL3 and PPARGC1A).

The generation of heat by adipocytes can be measured, by way of non-limiting example, using a calorimeter to detect heat generation by a population of cells as described by Clark et al. Biochem J. 1986 235:337-342 which is incorporated by reference herein in its entirety. The rate of growth and proliferation of a population of cells can be monitored by methods known to those of ordinary skill in the art. By way of non-limiting example, the degree of confluence or the number of cells in a population can be determined over a span of time. The expression of brown adipocyte marker genes (e.g. UCP1, ELOVL3, and PPARGC1A) can be measured by methods well known to those of ordinary skill in the art (see e.g. U.S. Pat. Nos. 7,319,933, 6,913,880,), including by quantitative RT-PCR as described in the Examples herein. By way of non-limiting example, the expression of brown adipocyte marker genes can also be measured by transient or stable transformation of a reporter construct into cultured cells. Candidate agents can be assayed for ability to increase expression of a reporter gene (e.g., GFP gene) under the control of a transcription regulatory element (e.g., promoter and/or enhancer sequence) of a brown adipocyte marker gene. An assay vector bearing the transcription regulatory element that is operably linked to the reporter gene can be transfected into a cell for assays of promoter activity. Reporter genes typically encode polypeptides with an easily assayed enzymatic or physical activity that is naturally absent from the host cell. Vectors expressing a reporter gene under the control of a transcription regulatory element of a marker gene can be prepared using routinely practiced techniques and methods of molecular biology (see, e.g., e.g., Sambook et al., supra; Brent et al., supra).

The number of mitochondria and their activity can be determined as described in the Examples herein. Briefly, mitochondria can be stained using MitoTracker and counted when the cells are viewed under a microscope. To determine the activity of mitochondria, cells can be incubated in prewarmed unbuffered DMEM medium (DMEM containing 2 mM GlutaMax, 1 mM sodium pyruvate, 1.85 g/L NaCl, and 25 mM glucose) for 1 hour. The oxygen consumption can be measured for example, using a XF24 Extracellular Flux Analyzer (Seahorse Biosciences). Mitochondrial biogenesis can be profiled by injecting perturbation drugs, 2 μM oligomycin, 0.5 μM CCCP (carbonyl cyanide p-trifluoromethoxyphenylhydrazone), and 5 μM antimycin A in succession as described in the Examples hlerein. Oxygen consumption rate (OCR) and extracellular acidification rate (ECAR) can be determined by plotting the oxygen tension and acidification by the cells of the medium in a chamber as a function of time and normalized by protein concentration (pmol/min/mg and mpH/min/mg), respectively. Higher OCR and ECAR rates indicate greater brown adipocyte activity and/or development.

The rate of glycerol release can be determined as described in the Examples herein. Briefly, day 21 adipocytes are incubated with 1-10 μM Isoproterenol or with 10 μM Forskolin. The culture media can be collected for glycerol measurement using the Free Glycerol reagent (Sigma #F6428). Protein concentrations used to normalize glycerol content are measured using the Bradford protein assay (BioRad). Glycerol release can be expressed in μ g glycerol per mg total protein, or the lowest value is set to 1. A higher rate of glycerol release in response to forskolin and isoproterenol is indicative of greater brown adipocyte activity, proliferation and/or development.

### Administration of Cells

One aspect of the invention relates to a method of providing brown adipocytes to a subject in need thereof. One aspect of the invention relates to the use of brown adipocytes, obtained according to the methods described herein, in therapy. In one embodiment, a therapeutically effective amount of brown adipocytes is provided, e.g. by differentiation of stem, progenitor or precursor cells as described herein. In some embodiments, the brown adipocytes are autologous. In some embodiments, the brown adipocytes are allogenic. In some embodiments, the brown adipocytes are syngeneic. In some embodiments, the brown adipocytes are xenogenic.

In some embodiments, administration of the brown adipocytes can occur within a relatively short period of time following differentiation in culture (e.g. 1, 2, 5, 10, 24 or 48 hours after differentiation). In some embodiments, the brown adipocytes can be cryogenically preserved prior to administration.

### Subject in Need of Treatment

Certain aspects of the methods described herein relate to administering brown adipocytes to a subject in need thereof. A subject in need of administration can be, but is not limited, to a subject with a higher than desired body-mass index (BMI) or higher than desired amount of white adipose tissue. This can include, but is not limited to, a subject diagnosed as having and/or at risk of having or developing type II diabetes, metabolic syndrome, insulin resistance, cardiac disease, early-onset myocardial infarction, osteoarthritis, gout, heart disease, gall bladder disease, fatty liver disease, sleep apnea, gall stones, and numerous types of cancer. Also envisioned is the treatment of patients who desire treatment for aesthetic reasons (i.e. to maintain a desired weight, BMI, or appearance) even if they are at a healthy weight or BMI prior to treatment.

Subjects in need of the brown adipocytes described herein can be identified by a physician using current methods of diagnosing, for example, a higher than desired BMI or diabetes. The diagnostic methods for diabetes include measuring the fasting blood glucose level (two measurements of a level higher than 126 mg/dL indicates a diagnosis of diabetes), the hemoglobin A1 c test (a result of 6.5% or higher indicates a diagnosis of diabetes) and the oral glucose tolerance test (a result of higher than 200 mg/dL after 2 hours indicates a diagnosis of diabetes). Symptoms of diabetes which characterize this condition and aid in diagnosis include, but are not limited to, polyuria, polydipsia, blurred vision, erectile dysfunction, pain or numbness in the feet or hands and unexplained weight loss.

Risk factors which can increase the likelihood of a subject being at risk of having or developing a higher than desired BMI include a high caloric intake, sedentary lifestyle, hypothyroidism and a family history of high BMI or obesity. Risk factors which can increase the likelihood of a subject being at risk of having or developing diabetes include, a higher than desired BMI, a high caloric intake, sedentary lifestyle, and a family history of diabetes.

### Dosage and Administration

As used herein, the term “administer” or “transplant” refers to the placement of cells into a subject by a method or route which results in at least partial localization of the cells at a desired site such that a desired effect is produced.

The brown adipocytes described herein can be administered in any manner found appropriate by a clinician and can include local administration, e.g. by injection of a suspension of brown adipocytes or, for example, by implantation of a preparation of brown adipocytes deposited or grown on or within an implantable scaffold or support. Implantable scaffolds can include any of a number of degradable or resorbable polymers, or, for example, a silk scaffold, among others. Suitable routes for administration of a brown adipocyte pharmaceutical composition include but are not limited to local administration, e.g. intraperitoneal, intracavity or subcutaneous administration.

The phrases “parenteral administration” and “administered parenterally” as used herein, refer to modes of administration other than enteral and topical administration, usually by injection, and includes, without limitation, intraperitoneal, intradermal, subcutaneous injection and infusion.

Administration can involve the use of needles, catheters and syringes suitable for injection, grafting cannula or surgical implantation. For example, the route of delivery can include open delivery through a standard blunt tip cannula (e.g. 14 gauge) inserted into the soft tissue through an appropriately placed incision.

The use of a combination of delivery means and sites of delivery are contemplated to achieve the desired clinical effect.

### Dosage

In one embodiment, a therapeutically effective amount of brown adipocytes as described herein is administered to a subject. A “therapeutically effective amount” is an amount brown adipocytes sufficient to produce a measurable improvement in a symptom or marker of the condition being treated. Actual dosage levels of brown adipocytes in a therapeutic composition can be varied so as to administer an amount of the brown adipocytes that is effective to achieve the desired therapeutic response for a particular subject. The selected dosage level will depend upon a variety of factors including, but not limited to, the activity of the therapeutic composition, formulation, the route of administration, combination with other drugs or treatments, severity of the condition being treated, the physical condition of the subject, prior medical history of the subject being treated and the experience and judgment of the clinician or practitioner administering the therapy. Generally, the dose and administration scheduled should be sufficient to result in slowing, and preferably inhibiting progression of the condition and also preferably causing a decrease in one or more symptoms or markers of the condition, e.g. obesity, diabetes, higher than optimal BMI, etc and their makers and symptoms. Determination and adjustment of a therapeutically effective dose, as well as evaluation of when and how to make such adjustments, are known to those of ordinary skill in the art of medicine.

The dosage of brown adipocytes administered according to the methods described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer another dose of cells, increase or decrease dosage, discontinue treatment, resume treatment or make other alteration to the treatment regimen. Where cells administered are expected to engraft and survive for medium to long term, repeat dosages can be necessary. However, administration can be repeated as necessary and as tolerated by the subject.

The dosage should not be so large as to cause substantial adverse side effects. The dosage can also be adjusted by the individual physician in the event of any complication. Typically, however, the dosage can range from 0.5 g to 500 g of brown adipocytes for an adult human. In some embodiments, the dosage can range from 1 g to 100 g for an adult human. In some embodiments, the dosage can range from 20 g to 70 g for an adult human. Effective doses can be extrapolated from dose-response curves derived from, for example, animal model test bioassays or systems.

Therapeutic compositions comprising brown adipocytes prepared as described herein thereof are optionally tested in one or more appropriate in vitro and/or in vivo animal models of disease, such as a mouse model of obesity, to confirm efficacy, evaluate in vivo growth of the transplanted cells, and to estimate dosages, according to methods well known in the art. In particular, dosages can be initially determined by activity, stability or other suitable measures of treatment vs. non-treatment (e.g., comparison treated vs. untreated animal models), in a relevant assay. Formulations are administered at a rate determined by the LD₅₀ of the relevant formulation, and/or observation of any side-effects of brown adipocytes as described herein at various concentrations, e.g., as applied to the mass and overall health of the patient. In determining the effective amount of brown adipocytes, the physician evaluates, among other criteria, the growth and volume of the transplanted cells and progression of the condition being treated.

The dosage can vary with the dosage form employed and the route of administration utilized.

With respect to the therapeutic methods described herein, it is not intended that the administration of brown adipocytes be limited to a particular mode of administration, dosage, or frequency dosing. All modes of administration are contemplated, including intramuscular, intraperitoneal, subcutaneous, or any other route sufficient to provide a dose adequate to treat the condition being treated.

### Pharmaceutical Formulations

In some embodiments, a pharmaceutical composition comprises brown adipocytes as described herein, and optionally a pharmaceutically acceptable carrier. The compositions can further comprise at least one pharmaceutically acceptable excipient.

The pharmaceutical composition can include suitable excipients, or stabilizers, and can be, for example, solutions, suspensions, gels, or emulsions. Typically, the composition will contain from about 0.01 to 99 percent, preferably from about 5 to 95 percent of cells, together with the carrier. The cells, when combined with pharmaceutically or physiologically acceptable carriers, excipients, or stabilizer, can be administered parenterally, subcutaneously, by implantation or by injection. For most therapeutic purposes, the cells can be administered via injection as a solution or suspension in liquid form.

The term “pharmaceutically acceptable carrier” refers to a carrier for administration of the brown adipocytes. Such carriers include, but are not limited to, saline, buffered saline, dextrose, water, glycerol, and combinations thereof. Each carrier must be “acceptable” in the sense of being compatible with the other ingredients of the formulation, for example the carrier does not decrease the impact of the agent on the treatment. In other words, a carrier is pharmaceutically inert and compatible with live cells.

Suitable formulations also include aqueous and non-aqueous sterile injection solutions which can contain anti-oxidants, buffers, bacteriostats, bactericidal antibiotics and solutes which render the formulation isotonic with the bodily fluids of the intended recipient. Aqueous and non-aqueous sterile suspensions can include suspending agents and thickening agents. The formulations can be presented in unit-dose or multi-dose containers.

### Parenteral Dosage Forms

Examples of parenteral dosage forms include, but are not limited to, solutions ready for injection, suspensions ready for injection, and emulsions. Parenteral dosage forms can be prepared, e.g., using bioresorbable scaffold materials to hold brown adipocyte preparations.

### Efficacy

Efficacy of treatment can be assessed, for example by measuring a marker, indicator, symptom or incidence of, the condition being treated (e.g. BMI, diabetes, etc.) as described herein or any other measurable parameter appropriate, e.g. brown fat cell numbers or mass. It is well within the ability of one skilled in the art to monitor efficacy of treatment or prevention by measuring any one of such parameters, or any combination of parameters.

Effective treatment is evident when there is a statistically significant improvement in one or more markers, indicators, or symptoms of the condition being treated, or by a failure to worsen or to develop symptoms where they would otherwise be anticipated. As an example, a favorable change of at least about 10% in a measurable parameter of a condition, and preferably at least about 20%, about 30%, about 40%, about 50% or more can be indicative of effective treatment. Efficacy for brown adipocytes as described herein can also be judged using an experimental animal model known in the art for a condition described herein. When using an experimental animal model, efficacy of treatment is evidenced when a statistically significant change in a marker is observed, e.g. degree of obesity.

### Kits

In one aspect, the invention is also directed to kits for promoting the differentiation of cells into brown adipocytes. A kit can comprise at least one agent that increases the level or activity of PPARγ2 and C/EBPβ. A kit does not comprise an agent which increases the level of PRDM16. Optionally, a kit can comprise a cell or population of cells which are to be differentiated into brown adipocytes. Optionally, a kit can comprise containers and/or media for the culture of the cells which are to be differentiated as well as the brown adipocytes which are differentiated according to the methods described herein.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments can perform functions in a different order, or functions can be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments can also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

This invention is further illustrated by the following examples which should not be construed as limiting.

Some embodiments of the technology described herein can be defined according to any of the following numbered paragraphs:

1. A method for promoting the differentiation of cells into brown adipocytes comprising;
a) contacting a population of cells with at least one agent that increases the level or activity of PPARγ2 and C/EBPβ; and
b) culturing the cells under conditions favorable for differentiation into brown adipocytes; wherein the method does not comprise contacting the cells with an agent which increases the level of PRDM16.

2. The method of paragraph 1, wherein the agent that increases the level or activity of PPARγ2 and C/EBPβ comprises a polynucleotide comprising a gene sequence that encodes a PPARγ2 and/or a C/EBPβ polypeptide.

3. The method of paragraph 1, wherein the agent that increases the level or activity of PPARγ2 and C/EBPβ comprises a PPARγ2 polypeptide and/or C/EBPβ polypeptide.

4. The method of paragraph 1, wherein the agent that increases the level or activity of PPARγ2 and C/EBPβ comprises a small molecule that increases the level or activity of PPARγ2 or C/EBPβ.

5. The method of paragraph 3, wherein the small molecule is a selected from the group consisting of:
a thiazolidinedione or a glitazar.

6. The method of paragraph 1, wherein the cells are selected from the group consisting of:
non-neuronal somatic cells, differentiated non-neuronal cells, fibroblasts, adipose-derived cells, adipose-derived stromal vascular cells, and stem or progenitor cells.

7. The method of paragraph 6, wherein the stem cells or progenitor cells are chosen from the group consisting of:
induced pluripotent stem cells, adipose-derived stem cells, adipose-derived mesenchymal stem cells, adipose progenitor cells, embryonic stem cells, and mesenchymal stem cells.

8. The method of paragraph 1, wherein said cells are initially provided by inducing a population of pluripotent stem cells to differentiate to a mesenchymal stem cell phenotype.

9. The method of paragraph 1, wherein the cells are human cells.

10. The method of paragraph 1, wherein the brown adipocytes are differentiated in vitro.

11. The method of paragraph 1, wherein the brown adipocytes are differentiated ex vivo.

12. The method of paragraph 1, wherein the rate of differentiation to brown adipocytes is at least 80%.

13. A method for promoting the differentiation of pluripotent stem cells into brown adipocytes comprising;
differentiating pluripotent stem cells into mesenchymal stem cells;
contacting the mesenchymal stem cells with at least one agent that increases the level or activity of PPARγ2 and C/EBPβ; and
culturing the cells under conditions favorable for the differentiation into brown adipocytes; wherein the method does not comprise contacting the cells with an agent that increases the level or activity of PRDM16.

14. A method for screening for agents that increase the development of brown adipocytes comprising;
contacting cells with at least one agent that increases the level or activity of PPARγ2 and C/EBPβ;
contacting the cells with an additional candidate agent; and
culturing the cells under conditions favorable for differentiation into brown adipocytes;
   wherein a candidate agent is identified as an agent that increases the development of brown adipocytes if the rate of proliferation or rate of differentiation of brown adipocytes is higher in the presence of the candidate agent.

15. A method for screening for agents that increase the activity of brown adipocytes comprising;
contacting cells with at least one agent that increases the level or activity of PPARγ2 and C/EBPβ;
culturing the cells under conditions favorable for differentiation into brown adipocytes; and
contacting the brown adipocytes with a candidate agent;
wherein a candidate agent is identified as an agent that increases the activity of brown adipocytes if a measure of brown adipocyte activity is higher in the presence of the candidate agent.

16. The method of paragraph 15, wherein the measure of brown adipocyte activity is the generation of heat.

17. The method of paragraph 15, wherein the measure of brown adipocyte activity is the rate of growth or proliferation of the adipocytes.

18. The method of paragraph 15, wherein the measure of brown adipocyte activity is selected from the group consisting of:
expression of brown adipocyte marker genes; measurement of mitochondrial number and activity; and glycerol release.

19. A method of providing brown adipocytes to a subject in need thereof comprising;
differentiating brown adipocytes from cells ex vivo according to the method of Paragraph 1; and
transplanting the brown adipocytes so differentiated into the subject.

20. The method of paragraph 19, wherein the cells are autologous.

21. A kit for promoting the differentiation of cells into brown adipocytes comprising;
at least one agent that increases the level or activity of PPARγ2 and C/EBPβ; and optionally, a population of cells;
   wherein the kit does not comprise an agent which increases the level of PRDM16.

22. Brown adipocytes obtained by a method according to any of claims 1 to 13.

23. Use of a brown adipocyte according to claim 22 in therapy.

### EXAMPLES

The utility of human pluripotent stem cells is dependent on efficient differentiation protocols that convert these cells into relevant adult cell types. Described herein is the robust and efficient differentiation of human pluripotent stem cells into white or brown adipocytes. As described herein, inducible expression of PPARG2 or PPARG2 combined with CEBPB in pluripotent stem cell-derived mesenchymal progenitor cells programmed their development, respectively, towards a white or brown adipocyte cell fate with efficiencies of 85% to 90%. These pluripotent stem cell-derived adipocytes retained their identity independent of transgene expression, could be maintained in culture for at least several weeks, expressed mature markers, and exhibited mature functional properties such as lipid catabolism and insulin-responsiveness. When transplanted into mice the programmed cells gave rise to ectopic fat pads with the morphological and functional characteristics of white or brown adipose tissue. These results indicate that the cells can be used to faithfully model human disease, as well as to treat conditions responsive to increased numbers of brown adipocytes.

### Example 1

### Results

In one approach, pluripotent stem cells, including, but not limited to iPSCs and naturally-occurring pluripotent stem cells are the starting material for the generation of brown adipocytes. Of course, any available source of mesenchymal stem or progenitor cells can be used where desired. MSCs derived from hiPSCs are used in the methods exemplified below.

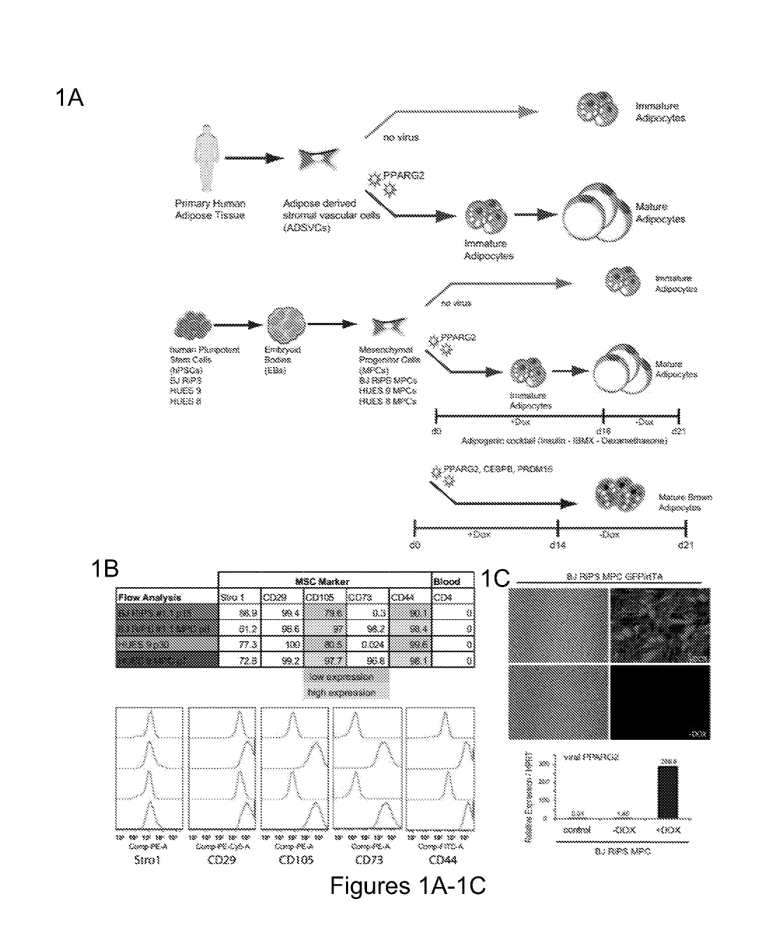

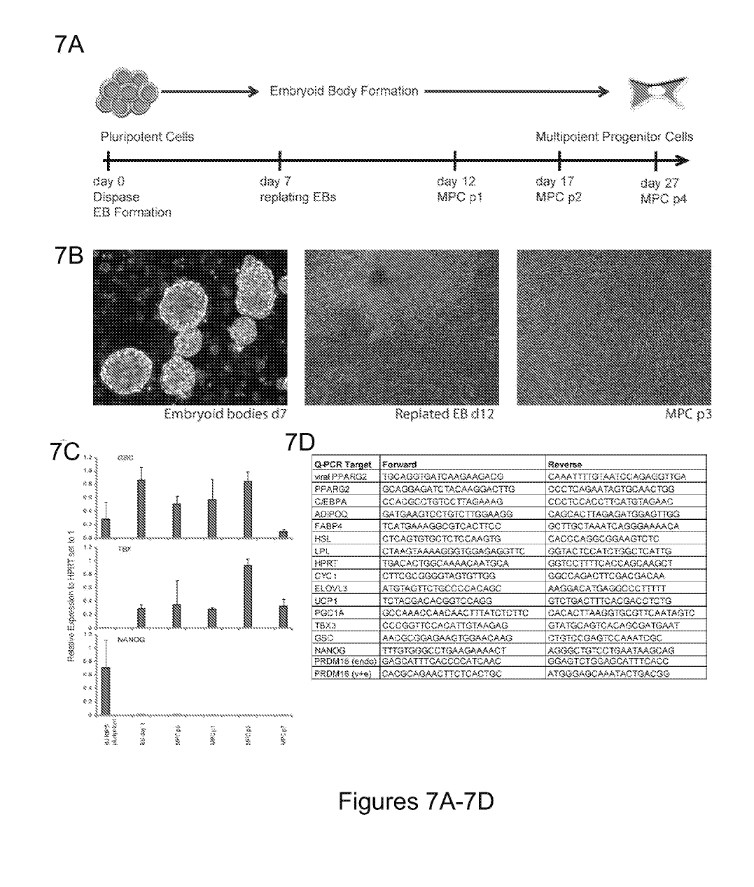

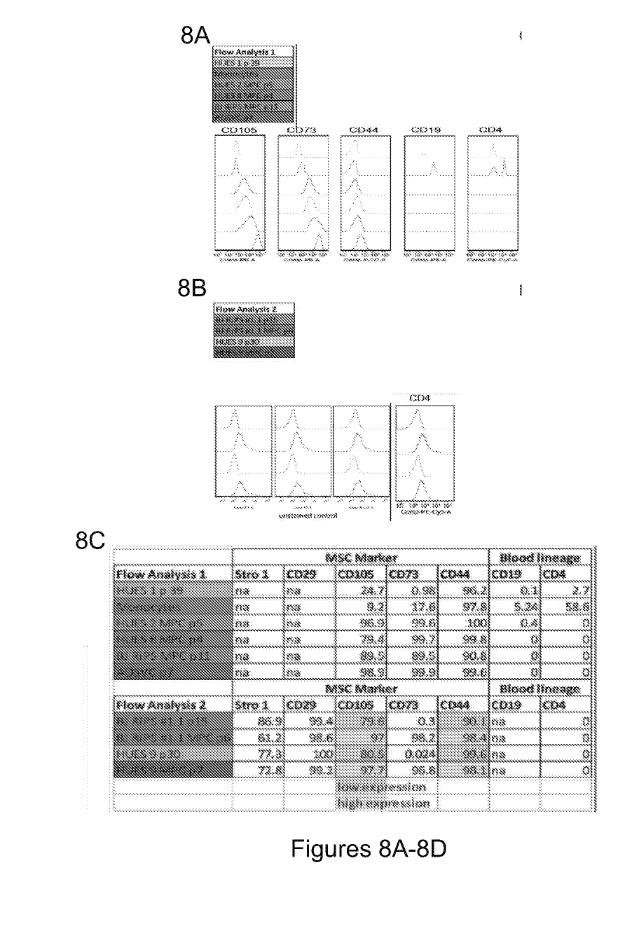

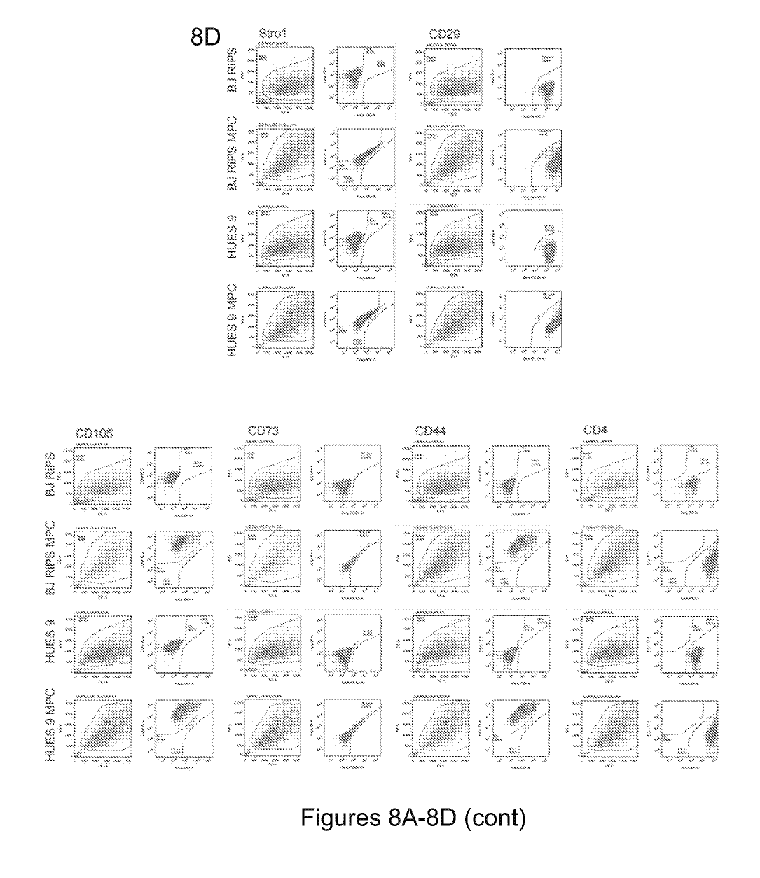

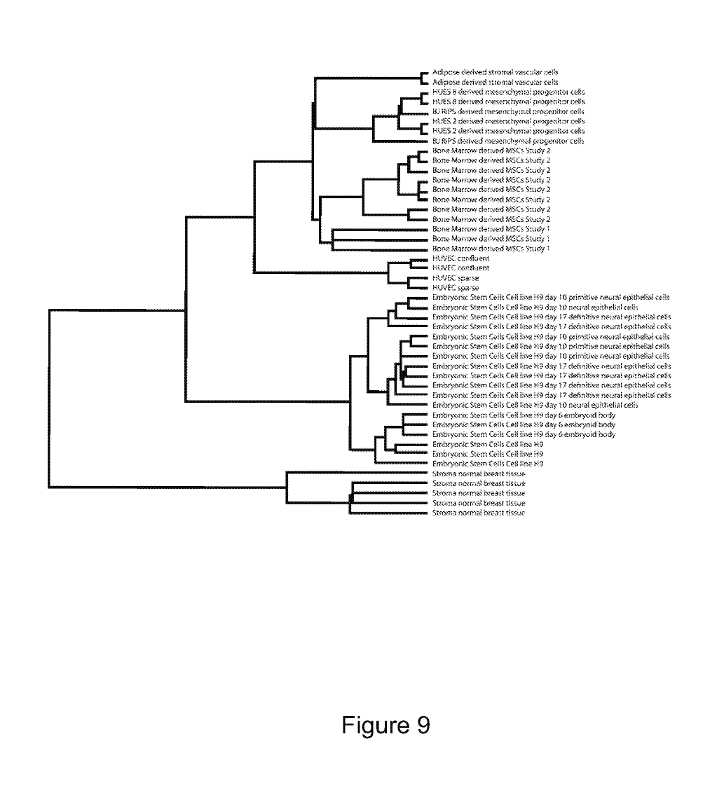

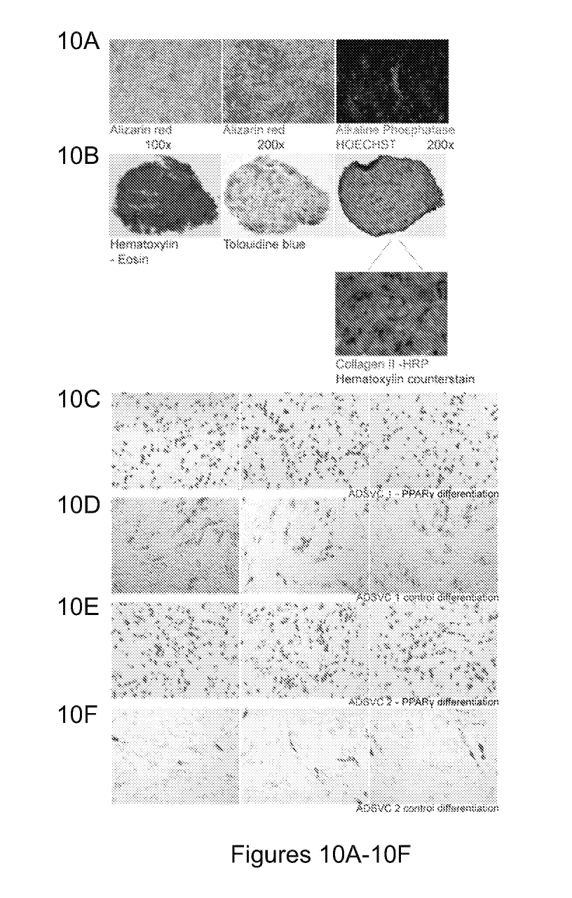

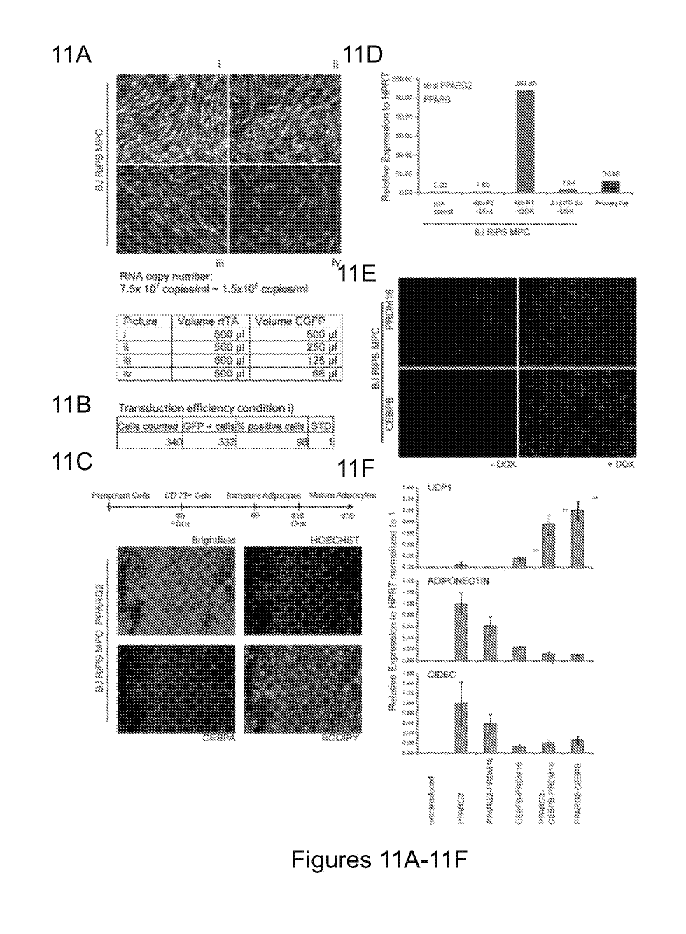

Differentiation of hPSCs to MPCs. Several protocols for generating mesenchymal stem cells (MSCs) or mesenchymal progenitor cells (MPCs) from hPSCs have been previously described^{30, 31}. Notably, these cells had the potential to form adipocytes. The derivation of (MPCs) from hPSCs was simplified as described herein (FIG. 1A; FIG. 7A). Three hESC lines and two iPSC lines³² were differentiated into embryoid bodies (EBs) that after two days in suspension culture displayed a characteristic rounded shape with defined and smooth borders. After ten days, these EBs were plated to adherent cell culture dishes, and fibroblast-like cells were observed growing from the EBs (FIG. 7B). The derived fibroblast-like cells were replicative and were capable of expansion for 10-12 passages. These cells were analyzed with flow cytometry for markers characteristic of a MPC fate—Strol, CD29, CD105, CD73, CD44—and negative control markers of the blood lineage CD19 and CD4 (FIG. 1B; FIGS. 8A-8D). More than 96% of the fibroblast-like cells expressed the CD73, CD29, CD44, and CD105; ADSVCs similarly expressed CD73, whereas hPSCs lacked the CD73 surface antigen entirely. Global transcriptional analysis of the derived cells showed high similarity to a variety of mesenchymal progenitor cell lines (FIG. 9 data not shown). It was possible to differentiate the fibroblast-like cells into osteoblasts and chondrocytes FIGS. 7C, 10A-10F) and adipocytes. In light of the characteristic markers and the trilineage differentiation results, the fibroblast-like cells are herein termed MPCs. Further it was possible to efficiently transduce these cells utilizing an inducible lentiviral system (FIGS. 1C, 11A-11C).

Differentiation of hPSCs to MPCs

It was hypothesized that efficient differentiation of hPSCs into adipocytes might be achieved by first establishing an intermediate fibroblast population wherein the effects of various adipogenic stimuli could be examined. Several protocols for generating MSCs or MPCs from hPSCs have been previously described^{31, 55}. For example, hPSCs can be first co-cultured with immortalized stromal cell lines, followed by the use of fluorescence-activated cell sorting (FACS) for cell surface antigens such as CD73 to isolate populations of MSCs/MPCs^{55, 56}. Alternatively, MSCs can be derived without a co-culture step^{33, 57}. As another alternative, MPCs can be derived by using FACS to isolate CD73+cells from a population of ES cell-derived neuronal cells. These MPCs are characterized by a uniform CD73 expression and, after additional culturing, most cells express CD73 and coexpress other surface markers characteristic of mesenchymal stem cell fate, including Stro-1, CD29, CD73, CD44, and, to a lower degree, CD105⁵⁸.

The expression levels of pluripotency genes and mesoderm development genes were analyzed at different stages during the differentiation protocol (FIG. 7C). The mesendoderm marker GOOSECOID (GSC) was detected in the pluripotent stem cells, which might be due to spontaneous differentiation of stem cells; however, GSC levels were observed to increase during differentiation. GSC is a transient developmental regulator, and consistent with that function GSC levels declined again after prolonged culture of the MPCs. The mesodermal marker T-box transcription factor 3 (TBX3) was absent in the pluripotent stage but was expressed during differentiation, and expression was maintained during all stages of the differentiation protocol. NANOG, a marker of pluripotency, was observed at very high levels in the pluripotent stage but rapidly diminished during differentiation.

Characterization of hPSC derived MPCs

To further assess the hPSC-derived MPCs, global gene expression profiles of these cells and ADSVCs were generated using a standard Affymetrix microarray (Human Genome U133 Plus 2.0 Array) and compared to a panel of expression profiles previously derived from a variety of tissue and cell types and deposited in the GEO database^{59,60,61,62} (GEO: GSE9940) (data not shown). The microarray results and GEO data were clustered using the Pearson correlation coefficient (r) and high reproducibility was found among replicates for all of our samples (r>0.97) and high similarity among the hPSC-derived MPCs (r=0.975 for cells derived from HUES 2, HUES 8, and BJ RiPS). Strikingly, the ADSVCs did not exclusively segregate with the hPSC-derived MPCs, but rather with a larger cluster that included the hPSC-derived MPCs as well as bone-derived MSCs (BMSCs) and MSCs deposited in GEO. These results suggest that the hPSC-derived MPCs are a subtype of MSCs along with BMSCs and ADSVCs. The MSC cluster (average r>0.95) was clearly independent from other clusters containing hESCs, hESCs-derived neuronal cell types, and various other cultured lines (e.g., HUVEC); all of these clustered separately from primary breast stromal tissue samples (FIG. 9). The results of our flow cytometry experiments were confirmed using the expression array data specific to mesenchymal surface markers (FIG. 7C).

The hPSC-derived MPCs were differentiated into osteoblasts and chondrocytes. To confirm the osteoblast identity the differentiated cells were stained with alizarin red and immunocytochemistry for alkaline phosphatase (FIG. 10A). Chondrocyte differentiation was confirmed by staining sectioned microspheres with hematoxylin and eosin and immunohistochemistry with an antibody against chondrocyte-specific collagen II (FIG. 10B). Tolouidine blue was used to stain glycosaminoglycans present in chondrocytes. The chondrocyte identity was also confirmed by immunohistochemistry with an antibody against chondrocyte-specific collagen II (FIG. 10B).

Efficient transduction of MPCs

To examine the efficiency of the lentiviral system described herein, MPCs were co-infected with Lenti-rtTA and a doxycyline-inducible enhanced green fluorescence protein (EGFP) lentivirus at four different viral concentrations (FIGS. 11A-11B). In the presence of doxycycline strong and robust expression of EGFP was observed, whereas in the absence of doxycycline green fluorescence was not detectable above background (FIG. 1C; fluorescence visible as light grey). The transduction efficiency of MPCs was approximately 98% (standard deviation +/−1%) as measured by the proportion of GFP+ cells upon infection with lentiviral concentrations of greater than 375 copies per cell (FIG. 11C).

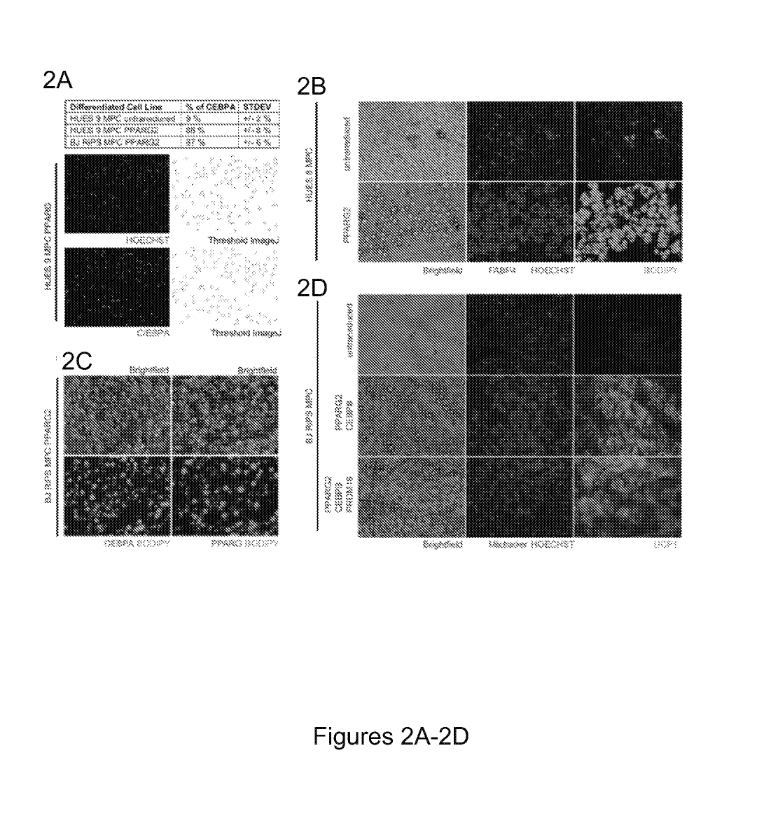

Differentiation of MPCs into white adipocytes. To determine whether the hPSC-derived MPCs could generate white adipocytes as previously reported^{30, 33}, their response to known inducers of adipogenesis—insulin, rosiglitazone, and dexamethasone was examined in comparison to human ADSVCs. After 21 days of exposure to a combination of these factors, a small percentage of MPCs and ADSVCs contained multiloccular lipid droplets resembling those often found in immature white adipocytes (FIGS. 2B, top panels and 10C-10F). ADSVC lines from distinct donors showed considerable variation in their ability to differentiate into white adipocytes, 10%-70%, as has been previously reported. The white adipocyte differentiation potential of MPC lines was also variable and was less than that of ADSVCs; only 3%-10% of MPCs differentiated into white adipocytes (FIG. 2A).

Programming MPCs into white adipocytes. In order to improve the differentiation of MPCs to white adipocytes the expression of a programming factor, namely PPARG2, a key regulator of adipogenesis^{11, 34, 35, 36} was temporally controlled. MPCs or ADSVCs were cultured in adipogenic media (“differentiated”) as well as either transduced (“programmed”) or not transduced (“unprogrammed”) with a lentiviral construct with a doxycycline-inducible promoter driving PPARG2 cDNA expression (Lenti-tet-PPARG2) and a construct constitutively expressing the reverse tetracycline transactivator (Lenti-rtTA)³⁷ allowing doxycycline-inducible expression of PPARG2 (FIG. 1C) along with doxycycline for 16 days, after which doxycycline was removed from the media and the cells cultured for an additional five days (FIG. 1A). After 21 days, the majority of PPARG2-programmed, differentiated MPCs displayed the typical morphology of mature white adipocytes, with a single large, well-defined lipid droplet, in contrast to unprogrammed, differentiated MPCs (FIGS. 2A-2C).

To determine the efficiency of white adipocyte differentiation, hPSC-derived adipocytes were immunostained with an antibody against CCAAT/enhancer-binding protein alpha (CEBPA; FIG. 2A). By counting the number of CEBPA-positive nuclei it was determined that on average 88% of PPARG2-programmed, differentiated MPCs were white adipocytes as compared to 9% of unprogrammed, differentiated MPCs (n=3). Programmed and unprogrammed MPCs were immunostained at day 21 of differentiation with antibodies against fatty-acid binding protein 4 (FABP4), PPARG, and CEBPA (FIGS. 2B, C). Strong staining of the cytoplasm with antibodies against FABP4 and nuclear staining with antibodies directed against PPARG2 and CEBPA was observed. The cells were also stained with BODIPY and displayed a morphology characteristic of mature white adipocytes, with most positively stained cells containing one large, predominant lipid droplet and a few surrounding small droplets. Thus, the data suggest that a 16-day pulse of PPARG2 expression is sufficient to permanently switch the fate of MPCs to the white adipocyte lineage.

Programmed adipocytes become independent of continuous transgene expression. While MPCs were efficiently programmed into white adipocytes by the exogenous expression of PPARG2, it was possible that these cells would initially adopt the characteristics of adipocytes but then would revert back to a non-adipocyte fate upon removal of PPARG2. To confirm that the hPSC-derived white adipocytes were not dependent on transgene expression, the cells were cultured for up to four additional weeks in the absence doxycycline. Twenty days after doxycycline withdrawal, these cells maintained the morphologic appearance of mature adipocytes, incorporated the fluorescent neutral lipid dye BODIPY [boron-dipyrromethene (4,4-difluoro-4-bora-3a,4a-diaza-s-indacene)], and stained positive for CEBPA, suggesting that the cells retained their white adipocyte identity (FIG. 11D), and (2) exhibited minimal expression of lentivirus-specific PPARG2 (FIG. 11E).

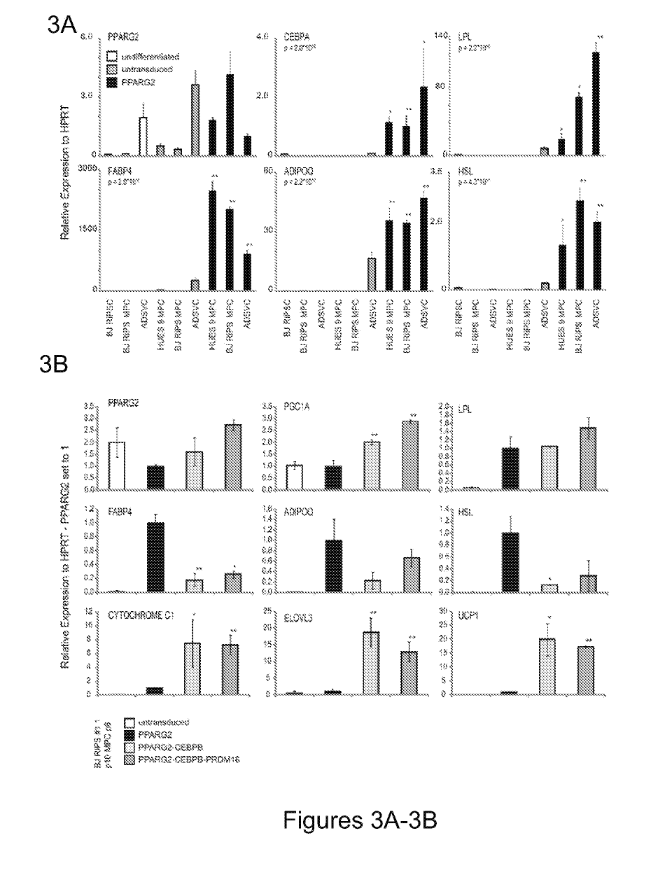

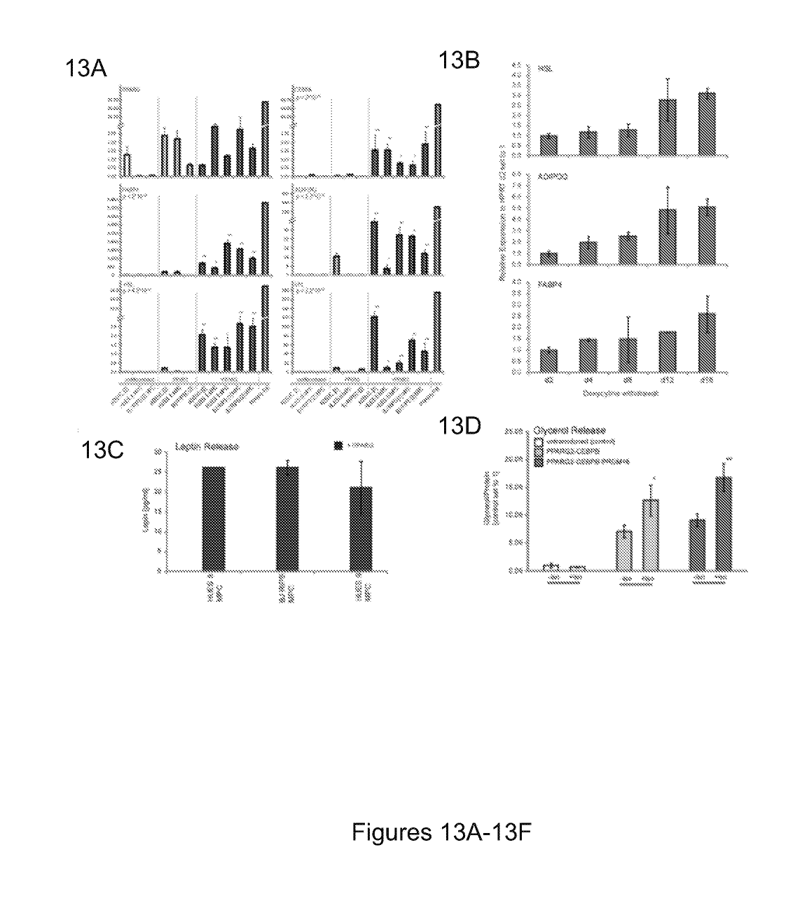

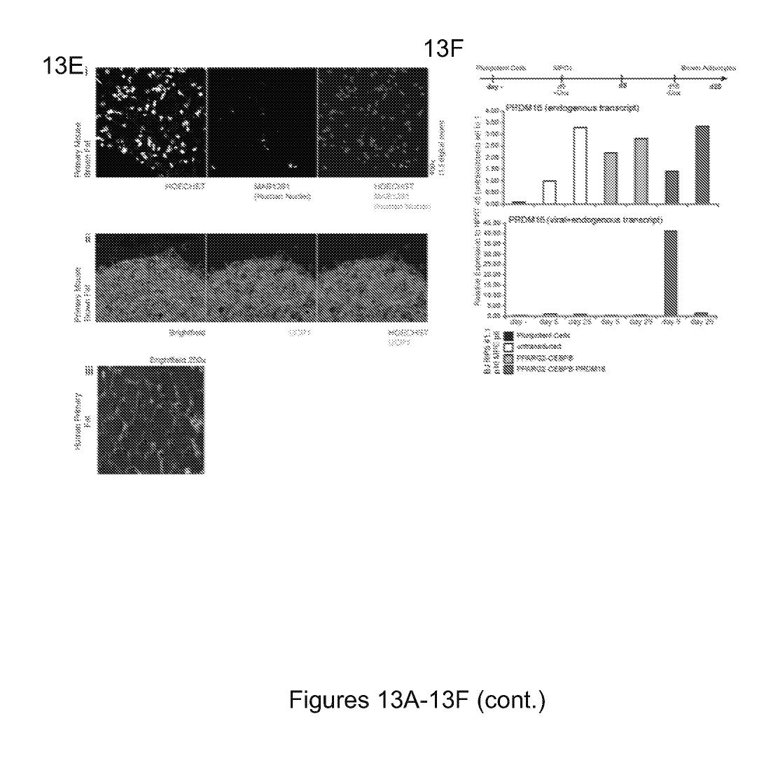

hPSC-derived adipocytes express mature markers. Quantitative reverse transcriptase-polymerase chain reaction (qRT-PCR) was perfomed for a panel of adipocyte marker genes, including lipoprotein lipase (LPL), hormone-sensitive lipase (HSL), adiponectin (ADIPOQ), FABP4, CEBPA, and PPARG2, in undifferentiated MPCs and ADSVCs and in unprogrammed or PPARG2-programmed, differentiated cells (n=3; FIGS. 3A and 13A). Adipocyte marker gene expression was absent in the undifferentiated cell lines with the exception of PPARG2, which was detected in ADSVCs as previously reported^{38, 39, 40}. Low levels of these genes were expressed in unprogrammed, differentiated MPCs and ADSVCs. In contrast, PPARG2-programmed, differentiated MPCs and ADSVCs at day 21 exhibited high levels of expression of all of the adipocyte marker genes, consistent with a mature white adipocyte fate.

The expression signature of PPARG2-programmed white adipocytes was compared with PPARG2-CEBPB-programmed and PPARG2-CEBPB-PRDM16-programmed brown adipocytes (n=3; FIG. 3B). Gene expression was similar for the endogenous PPARG2 gene and LPL. As expected, programmed white adipocytes showed higher expression of the white adipocyte markers ADIPOQ, HSL, and FABP4 compared to programmed brown adipocytes; programmed brown adipocytes showed higher expression of the brown adipocyte markers UCP1 and ELOVL3, PGC1A a major regulator of mitochondrial biogenesis as well as the mitochondrial marker CYC1 (cytochrome C1)⁴¹. Notably, these results were independent of transgene expression (FIG. 11E), confirming that the adipocyte markers observed were the result of an endogenous cellular program.

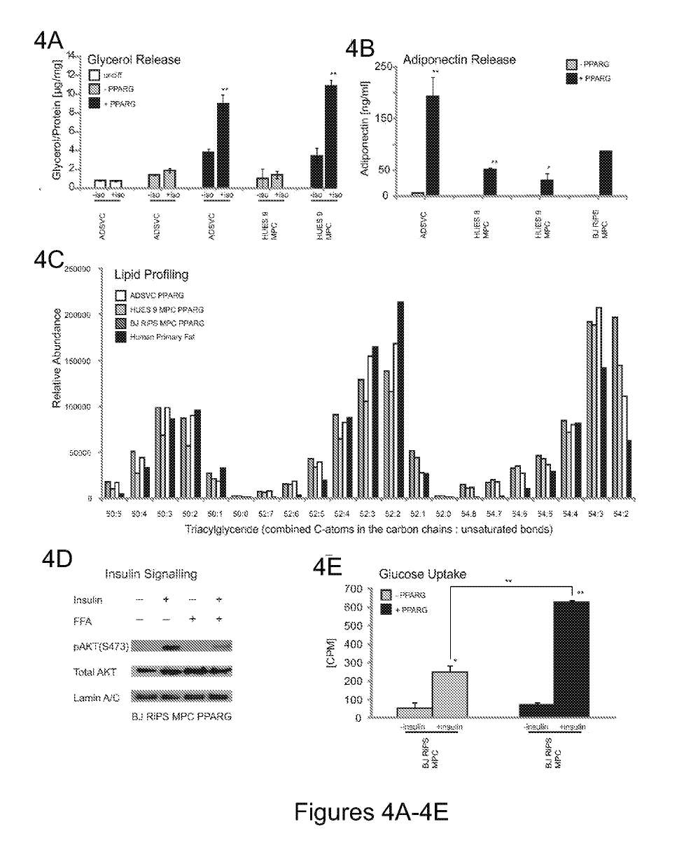

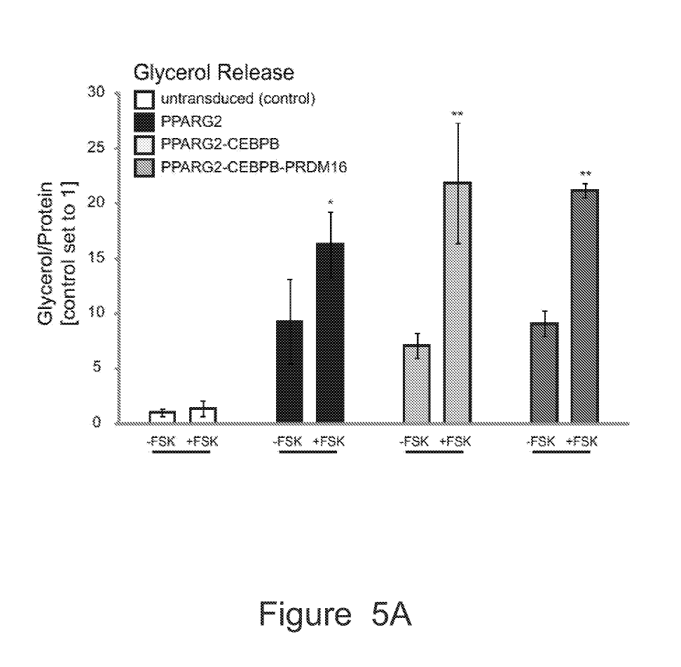

Programmed white adipocytes display a transcriptional signature similar to primary adipocytes. To evaluate how closely the hPSC-derived white adipocytes resembled primary white adipocytes on a genome-wide scale, transcript profiles were generated and analyzed from undifferentiated and PPARG2-programmed, differentiated ADSVCs and MPCs in addition to primary fat on two independent array platforms (FIGS. 4A-4B and data not shown). Global transcriptional profiling of PPARG-programmed adipocytes. ADSVCs, HUES 9 MPCs, and BJ RiPS MPCs either not exposed to adipogenic media (undifferentiated) or cultured with adipogenic media and transduced with lenti-PPARG (+PPARG), (PPARG2-CEBPB) or (PPARG2-CEBPB-PRDM16) were compared to primary adipocytes using Affymetix 1.0 ST microarrays and Agilent GE G3 microarrays. Unsupervised clustering by the Pearson correlation coefficient for all the expressed genes (˜20,000) resulted in two primary clusters: (1) PPARG2-programmed white adipocytes, derived from both MPCs and ADSVCs, and primary white fat (r=0.95); and (2) undifferentiated ADSVCs and MPCs (r=0.88). To determine the most significant changes in gene expression between the programmed/primary adipocytes and undifferentiated populations (ADSVCs, MPCs) the Significance Analysis of Microarrays (SAM) algorithm was used to call 2,136 differentially expressed genes using a 5% local false discovery rate (ADSVCs, HUES 9 MPCs, and BJ RiPS MPCs either not exposed to adipogenic media (undifferentiated) or cultured with adipogenic media and transduced with lenti-PPARG2 (+PPARG2) were compared to primary adipocytes using Affymetix 1.0 ST microarrays; data not shown)⁴².

A panel of adipocyte-specific genes was examined and expression was found to be higher in the programmed/primary white adipocytes in comparison to the undifferentiated cells (data not shown). Overall, the striking similarity of the primary white fat samples with the programmed white adipocytes (r=0.95, r=0.95) suggests that PPARG2 induction is a reliable and reproducible methodology for the derivation of homogeneous adipocytes from different hPSC types.

Transcript profiles from undifferentiated and PPARG2-programmed, differentiated ADSVCs and MPCs in addition to primary fat on two independent array platforms (Affymetrix HuGene-1_{—}0-st-v1r.4 and Agilent G3 Human GE) were generated and analyzed (data not shown).

Both platforms yielded highly concordant results in comparison of primary white adipocytes and PPARG2-programmed samples against undifferentiated samples using the Significance Analysis of Microarrays (SAM) algorithm to rank genes present on both platforms (Spearman rank correlation=0.84). Furthermore, overlap of differentially expressed genes at a 5% false discover rate (FDR) between both platforms was extremely high (p<10-300, hypergeometric test). For the following analyses and figures, the Affymetrix data is relied upon.

The most significant changes in gene expression between the programmed/primary adipocytes and undifferentiated populations (ADSVCs, MPCs) were determined. To that end the Significance Analysis of Microarrays (SAM) algorithm was used to call 2,136 differentially expressed genes using a 5% local false discovery rate (data not shown)¹¹ Cluster analysis by the Pearson correlation coefficient for this subset of genes clearly distinguished the programmed/primary white adipocytes (r=0.95 within the cluster) from the undifferentiated cells (r=0.8 within the cluster) as evidenced by the lower correlation between the two groups (r=0.49 for all samples) (data not shown).

To test if the 2,136 differentially expressed genes were consistent with adipogenesis, Gene Ontology (GO) term analysis was performed to uncover enriched functionally related gene sets from a collection of annotation databases using the DAVID Bioinformatics Resource^{64, 65} (data not shown). For genes upregulated in the adipocyte group compared to the undifferentiated cells, the most prominently enriched gene sets included annotations for PPARG2 signaling pathways as well as metabolism and genesis of fatty acids, and lipids (P<10⁻⁶, 10⁻⁹, and 10⁻¹⁰, respectively). This result further confirms that PPARG2 programmed adipocytes share the molecular signature of primary fat tissue and that these adipogenic genes are responsible for the two distinct clusters observed.

Detailed analyses of the genes which displayed the greatest differences between PPARG2-programmed adipocytes and primary fat indicated that many of these genes could be attributed to the various non-adipocyte cell types present in adipose tissue, including red blood cells (hemoglobin), leukocytes (major histocompatibility complex, class II, macrophage expressed 1, neutrophil cytosolic factors, lymphocyte cytosolic factors, immunoglobulins, and CD4), vascular endothelium (von Willebrand factor), and mesenchymal cells (lymphatic vessel endothelium hyaluronan receptor). To formalize these observations, the SAM algorithm was employed to determine significant differences in gene expression between primary fat and the PPARG2-programmed adipocytes for GO term analysis. Indeed, enriched gene sets upregulated in primary fat included genes known to play a role in immune defense (data not shown).

When adipocyte-specific genes were examined, such as adipocyte-specific adhesion molecule, resistin, cell death-inducing DFFA-like effector c, adipogenin, and angiotensinogen, it was found that there were no statistically significant differences in expression between primary fat and PPARG2-programmed adipocytes. Interestingly, some genes associated with de novo fatty acid synthesis, such as acyl-CoA synthetases, perilipin 3, and patatin-like phospholipase domain containing three proteins were higher in PPARG2-programmed adipocytes than primary fat. In addition, a few genes were higher in primary fat versus PPARG2-programmed adipocytes, including leptin, neuropeptide Y receptors, and the insulin receptor substrate¹.

Kinetics of programming adipocytes. Further, the kinetics of PPARG2-programmed differentiation into adipocytes was investigated. To that end the cells were differentiated for 21 days and doxycycline withdrawn at different time points. All conditions were analyzed at day 21 and the gene expression of adipogenic markers by qRT-PCR was examined (FIG. 13B). Even relatively short-term overexpression of PPARG2 increased the amount of adipogenesis. However, the adipogenic expression of markers generally increased over longer exposures. For the markers HSL and ADIPOQ, a plateau was reached after 14 days of doxycycline administration. FABP4 showed the highest expression after 16 days of doxycycline administration. In conclusion, a 12-16 day pulse of doxycycline appears sufficient to efficiently program hPSC-derived MPCs into white adipocytes.

Programming MPCs into brown adipocytes. The programming approach to achieve differentiation of MPCs was extended into brown adipocytes. PRDM16 has previously shown to convert murine myoblasts into brown adipocytes¹⁸, and a combination of CEBPB and PRDM16 to convert mouse cells and human fibroblasts into adipocytes with brown characteristics¹⁹. Accordingly, to generate brown adipocytes, MPCs were transduced with combinations of doxycycline-inducible lentiviral constructs (FIG. 11E) encoding the transcription factors PPARG2, CEBPB, and PRDM16 and differentiated in doxycyline-containing adipogenic medium. Generally, doxycycline was removed from the media after 14 days in culture and the cells cultured for an additional 7 days (FIG. 1A). After 21 days, striking morphological differences between PPARG2-programmed (white) adipocytes and PPARG2-CEBPB-programmed or PPARG2-CEBPB-PRDM16-programmed (brown) adipocytes were observed; programmed brown adipocytes generally had a multilocular lipid droplet morphology, and very few cells displayed the monolocular appearance noted in programmed white adipocytes (FIGS. 2B-2D). Programmed brown adipocytes were stained for UCP1 and strong staining of the cytoplasm, with almost no staining in unprogrammed cells was noted. Programmed brown adipocytes were further stained with the mitochondrial marker MitoTracker; strong staining in the cytoplasm, with a more diffuse morphology and much less staining in unprogrammed cells was observed (FIG. 2D). These findings were consistent with a brown adipocyte cell fate and distinguished the programmed brown adipocytes from white adipocytes.

Programming MPCs into brown adipocytes. Three transcription factor combinations showed a statistically significant increase in the expression of UCP1 (n=3; FIG. 11F). This included the previously reported transcription factor combination (CEBPB-PRDM16) as well as two others (PPARG2-CEBPB and PPARG2-CEBPB-PRDM16). This experiment was repeated independently three times and the combinations that included the programming factor PPARG2 showed a much higher efficiency in inducing UCP1 expression in the adipocytes. The cells were analyzed for specific markers preferentially expressed in white adipocytes, adiponectin and cell death-inducing DFFA-like effector c (CIDEC). The expression of ADIPOQ and CIDEC was similar among the three transcription factor combinations and, notably, lower than that seen in white adipocytes programmed with PPARG2 alone. Based on this observation, further work focused on two of the transcription factor combinations (PPARG2-CEBPB and PPARG2-CEBPB-PRDM16).

Forced expression of PRDM16 is not necessary for the programming of hPSC-derived MPCs into brown adipocytes. To confirm the surprising result that PRDM16 expression is relatively low in programmed brown adipocytes qRT-PCR was performed using a primer that detects only endogenous PRDM16 as well as a primer that detects viral and endogenous PRDM16. No obvious differences were found in endogenous PRDM16 expression between differentiated untransduced MPCs and PPARG2-CEBPB or PPARG2-CEBPB-PRDM16-programmed adipocytes (FIG. 13F). Endogenous PRDM16 was almost undetectable in pluripotent control cells. Untransduced MPCs, PPARG2-CEBPB or PPARG2-CEBPB-PRDM16-programmed adipocytes were analyzed at day 5 and day 25 of differentiation. In all conditions endogenous PRDM16 was expressed at day 5 of differentiation and showed an increase of expression at day 25. PRDM16 expression was comparable between untransduced and programmed cells (Top panel). Next a primer that detects viral and endogenous PRDM16 expression was utilized. At day 5 of differentiation in medium containing doxycyline, there was a 40 fold increase in PRDM16 expression in the PPARG2-CEBPB-PRDM16-programmed adipocytes (bottom panel). Doxycycline was removed from the medium and the PRDM16 expression dropped to comparable low levels in all conditions. This indicates that forced PRDM16 expression is not necessary for the differentiation of hPSC-derived MPCs into brown adipocytes. It is possible that the PRDM16 expression noted in untransduced differentiated MPCs in sufficient to govern brown adipocyte differentiation or that the differentiation is not relying on PRDM16 in the human in-vitro system described herein.

Programmed brown adipocytes display a distinct signature from white adipocytes. To assess the programmed brown adipocytes at the transcriptome level, transcript profiles from PPARG2-CEBPB-programmed and PPARG2-CEBPB-PRDM16-programmed adipocytes were generated and compared with transcript profiles from primary white adipose tissue in addition to undifferentiated and PPARG2-programmed, differentiated ADSVCs and MPCs (data not shown). Hierarchical clustering with Pearson correlation of 2,869 differentially expressed genes revealed that all of the adipose samples (programmed brown, programmed white, and primary white) cosegregate separately from the undifferentiated samples (ADSVCs and MPCs) (r=0.86). Importantly, within the adipose cluster, the programmed brown adipocytes (r=0.95) cluster distinctly from the programmed and primary white adipose (r=0.90).

A panel of genes that are associated with brown fat or white fat identity was further analyzed (data not shown; white adipocytes, primary white adipose tissue, undifferentiated MPCs; and PPARG2-CEBPB-PRDM16 programmed MPCs (brown adipocytes) were compared on the Agilent G3 Human GE array platform)^{41, 43, 44}. All of the genes that have been described as brown adipose tissue markers (UCP1, CYC1, NDUFA11, NDUFA13, CMT1A, ELOVL3, DIO2, LHX8, COX8A, and CYFIP2) show the highest expression in programmed brown adipocytes, with the only outlier being endogenous PRDM16, which showed the highest expression in primary and programmed white adipocytes. All differentiated cells expressed general adipocyte markers (CIDEC, PLIN1, FABP4) at much higher levels than undifferentiated cells. Genes associated with white fat identity (DPT and INHBB) were most highly expressed in programmed and primary white adipocytes.

To confirm the low expression of PRDM16 in programmed brown adipocytes qRT-PCR was performed using a primer that detects only endogenous PRDM16 as well as a primer that detects viral and endogenous PRDM16. No obvious differences in endogenous PRDM16 expression was found between differentiated untransduced MPCs and PPARG2-CEBPB or PPARG2-CEBPB-PRDM16-programmed adipocytes (FIG. 13F).

Programmed white adipocytes exhibit mature functional properties. To investigate the functional capabilities of hPSC-derived white adipocytes, a key property of mature white adipocytes, the ability to perform lipolysis, was measured via the release of glycerol after exposure to the beta-adrenergic agonist Isoproterenol in undifferentiated ADSVCs and programmed or unprogrammed, differentiated ADSVCs and MPCs (n=3; FIG. 4A). Only the programmed, differentiated cells responded to isoproterenol treatment with the breakdown of triglycerides and release of glycerol (ADSVCs: P=0.01; MPCs: P=0.002). The hormone adiponectin, previously shown to regulate glucose and fatty acid catabolism and produced by adipocytes^{45, 46} was assessed, using an enzyme-linked immunosorbent assay (ELISA). High levels of secreted adiponectin were observed from all PPARG2-programmed cell lines, including several differentiated hPSC-derived MPC lines, with low levels from control cell lines (n=3,; FIG. 4B). Utilizing a similar ELISA assay, low levels of leptin secretion (25 pg/ml) into the medium from PPARG2-programmed cells were observed (FIG. 13C).

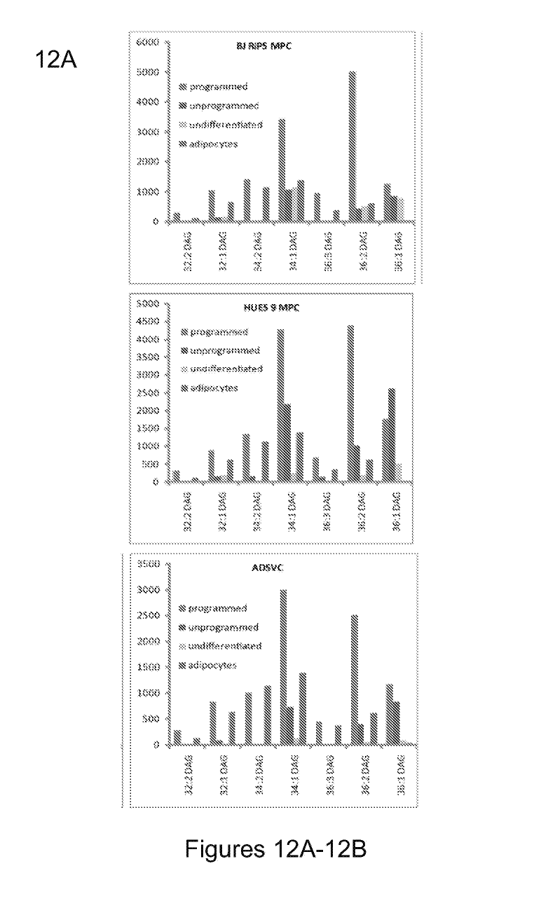

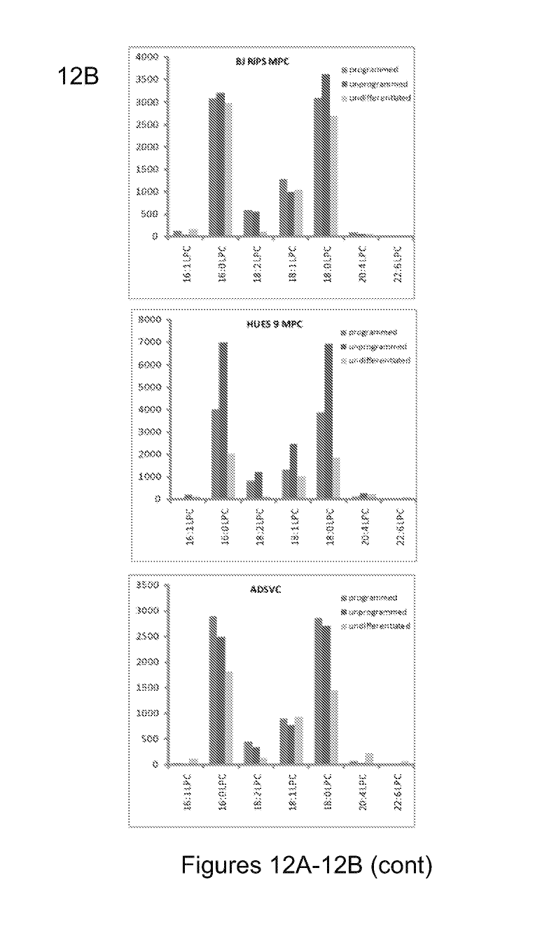

De novo fatty acid synthesis and storage in hPSC-derived adipocytes was characterized using a tandem mass spectroscopy lipidomics platform^{47, 48}. The cellular lipid content of ADSVC- and hPSC-derived white adipocytes programmed with PPARG2 expression was compared the findings to lipid profiles obtained from primary human white fat (FIG. 4C). Spearman correlation coefficients (SCC) were analyzed across all triglyceride species, and PPARG2-programmed, differentiated cells were much more similar to primary white adipocytes (SCC 0.75-0.87) than either unprogrammed, differentiated cells (SCC 0.41-0.62) or undifferentiated cells (SCC 0.21-0.32) (FIGS. 12A-12B; data not shown). Overall, remarkable congruence was observed among the lipid profiles of programmed white adipocytes and primary white adipose tissue, especially with respect to triglycerides, which represent the most abundant lipids in mature white adipocytes.

Lipid analysis. Consistent with increased expression of genes involved in de novo fatty acid synthesis, higher levels of some short-chain fatty acids were observed in programmed white adipocytes than in primary white adipose (data not shown; several long-chain triacylglyceride species, of a size range between 42:0 to 60:11, were measured in each cell type). The diacylglycerol content of programmed white adipocytes was also slightly altered as compared to primary white adipose tissue (FIG. 12A), which could be the result of either de novo synthesis or enhanced lipolysis. Membrane lipids, such as phosphatidylcholines, were similar in all cell types analyzed (FIG. 12B).

Finally, it was determined whether the programmed white adipocytes respond to insulin in a manner similar to mature primary white adipocytes. Insulin signaling of programmed white adipocytes was analyzed in the absence and presence of free fatty acids (FFA), an established contributor to induce insulin resistance in white adipocytes⁴⁹. Insulin response was examined by assessing protein expression levels of AKT and phosphorylated AKT [pAKT(S473)] (FIG. 4D). In the basal starved state no pAKT(S473) product was detected; administration of insulin robustly upregulated pAKT(S473). Coadministration of FFA reduced the insulin-induced phosphorylation of AKT, demonstrating that the hPSC-derived adipocytes appropriately model FFA-induced insulin resistance. The uptake of [3H]-2-deoxy-D-glucose was analyzed (FIG. 4E) and indicated that the basal level of glucose uptake was similar between PPARG2-programmed and unprogrammed cells. After exposure to insulin, a significant increase in the glucose uptake was noted in both cell types (P=0.05 for unprogrammed cells, P=0.0007 for programmed cells) but was substantially higher in the PPARG2-programmed cells (P=0.009). Taken together, these results validate programmed white adipocytes as a model of insulin response and insulin resistance.

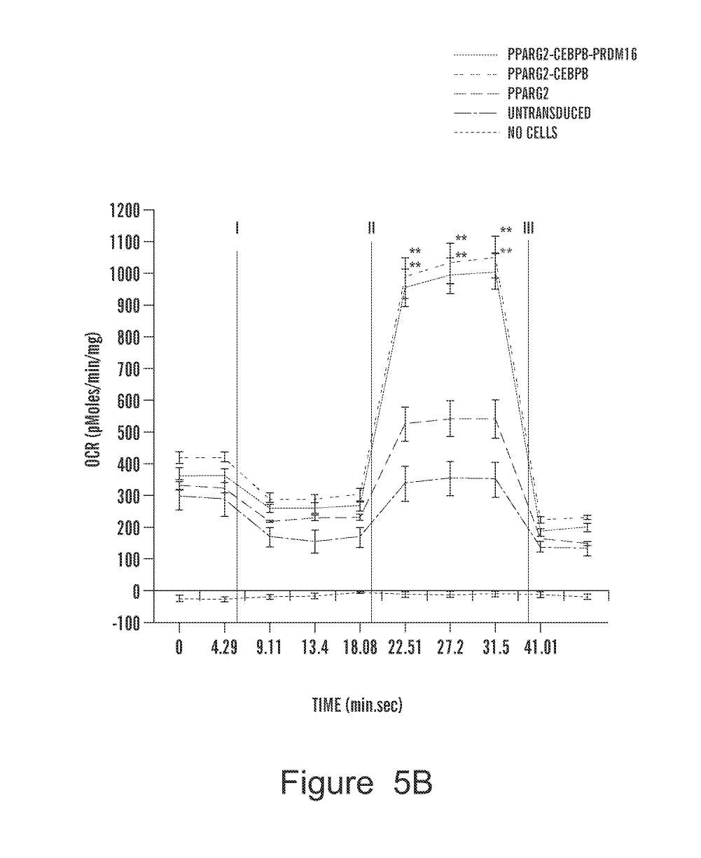

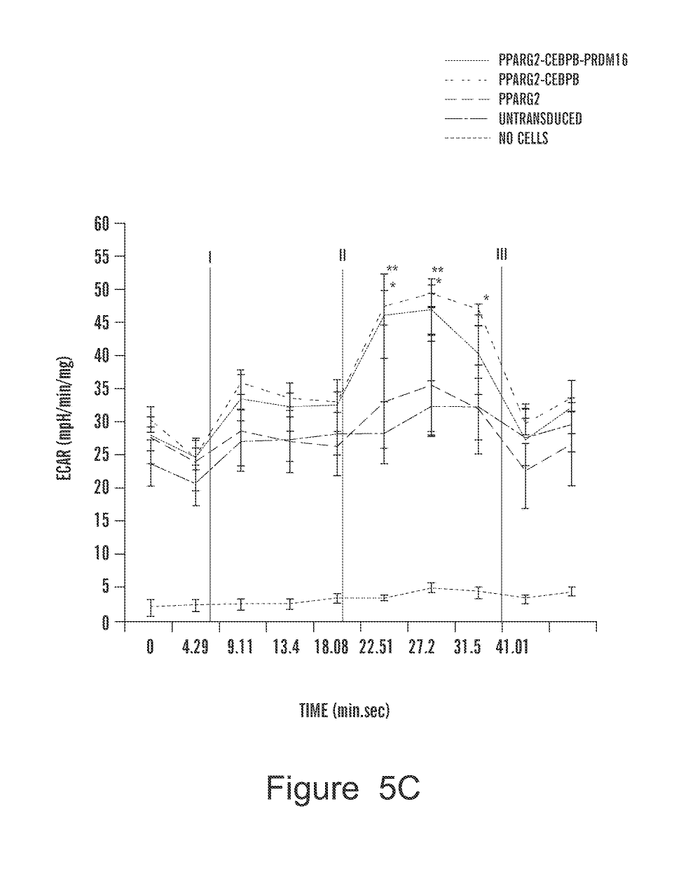

Programmed brown adipocytes exhibit mature functional properties. The characteristic function of brown adipocytes is thermogenesis, driven by the catabolic breakdown of lipids. The release of glycerol from our various cells was measured to determine the extent of lipolysis upon exposure to forskolin. As expected, both the white and brown hPSC-derived adipocytes described herein displayed a significantly higher release of glycerol in response to forskolin treatment (FIG. 5A) compared to unprogrammed cells. For the programmed brown adipocytes, the response to the beta-adrenergic agonist isoproterenol was determined and a significant increase in glycerol release compared to unprogrammed cells was observed (FIG. 13D). To further distinguish between white and brown adipocytes at a functional level, the characterization was extended to the mitochondrial function of our cells. The oxygen consumption rate (OCR) and extracellular acidification rate (ECAR) was determined using an extracellular flux analyzer (FIG. 5B-5C); the basal OCR and ECAR rates were highest in the programmed brown adipocytes. Compounds were then injected that modulate mitochondrial function sequentially and the effect on OCR and ECAR was measured after the addition of each compound. Oligomycin was first administered to determine ATP turnover and the degree of proton leak. At baseline, the programmed brown adipocytes showed slightly elevated levels of proton leak compared to unprogrammed cells. After the addition of the electron transport chain decoupler carbonyl cyanide m-chlorophenylhydrazone (CCCP), the maximal respiratory capacity was measured. Programmed brown adipocytes showed significantly higher levels of OCR and ECAR compared to the unprogrammed cells, whereas programmed white adipocytes did not. Finally, antimycin was administered to inhibit the flux of electrons through complex III and prevent oxygen consumption by the cytochrome c oxidase in the mitochondria. The OCR for all cell types dropped to post-oligomycin levels. Taken together, these results confirm that programmed brown adipocytes have significantly increased mitochondrial activity, an important functional characteristic of brown adipose tissue.

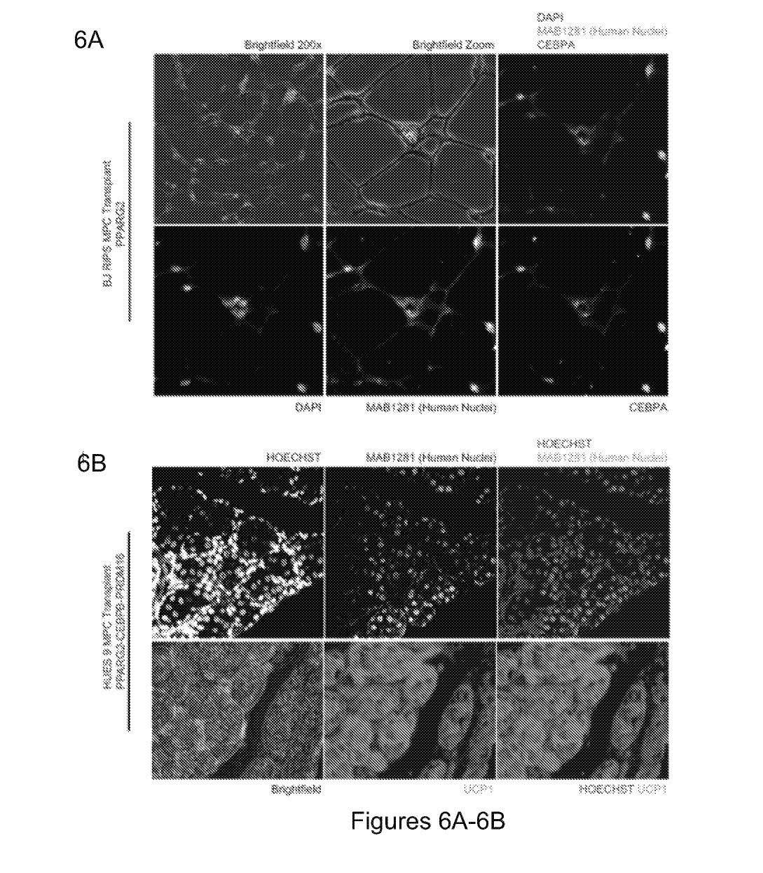

In vivo transplantation of hPSC-derived adipocytes. hPSC-derived brown and white adipocytes, as well as control 3T3-F442A cells, were administered subcutaneously to immunocompromised Rag2-/Il2rg-/- mice to study the function of the cells in vivo. The transplants were harvested and histologically examined after 4-6 weeks; importantly, no teratomas were observed (n=20). The extracorporeal origin of the harvested cells was confirmed by immunohistochemical staining with MAB1281, a human-specific nuclear antibody that does not stain nuclei in mouse cells (FIGS. 6A-6B and 13E (i)). Programmed white adipocytes stained positively for the adipocyte marker CEBPA whereas programmed brown adipocytes were positive for UCP1 (FIG. 6A-6B) in adjacent slides. Transplanted programmed white and brown adipocytes displayed morphology characteristic of primary adipocytes. Finally, the white and brown adipocyte transplants were assessed by fluordeoxyglucose (18FDG) uptake followed by positron emission tomography-computed tomography (PET-CT), a technique that has been used to detect brown adipose tissue in adult humans^{13,15,16,50}. Consistent with brown adipocytes acting as a “glucose sink,” the transplanted hPSC-derived brown adipocytes were highly FDG-avid and exhibited a clear PET signal as compared to hPSC-derived white adipocytes (data not shown).

In summary, described herein are efficient protocols to generate white and brown adipocytes from stem and progenitor cells, including hPSCs. Brown adipocytes show distinct morphology and gene expression profiles from white adipocytes, and each type of cell demonstrated functional properties that are characteristic of the corresponding tissue types in vivo. Transplantation of the cells into mice yielded tissues with functional and morphological similarities to primary white and brown adipose tissues. Taken together, these experiments confirm the identity and maturity of the hPSC- derived white and brown adipocytes and indicate that the cells can be used to faithfully model human disease, among other uses.

### Experimental Procedures

Maintenance of pluripotent cells, generation of mesenchymal progenitor cells (MPCs), and adipocyte differentiation. hESCs and hiPSCs were cultured feeder free on Geltrex (Invitrogen) in the chemically defined medium mTESR1 (Stem Cell Technologies). To induce differentiation of hESCs and hiPSCs into embryoid bodies (EBs), hPSCs were disaggregated with dispase into small clumps containing 5-10 cells and transferred to low-adhesion plastic 6-well dishes (Costar Ultra Low Attachment; Corning Life Sciences) in growth medium containing DMEM, 15% FBS, and 1% Glutamax. After 7 days in suspension culture, EBs were collected and replated on gelatin-coated 6-well dishes in medium containing DMEM, 10% FBS, and 1% Glutamax. After cells reached confluency (in approximately 5 days) they were trypsinized (0.25% trypsin) and replated on cell culture dishes containing MPC growth medium containing DMEM, 15% FBS, 1% Glutamax, and 2.5 ng/ml bFGF (Aldevron). Cells were passaged with a 1:3 split ratio and used for differentiation experiments prior to passage 8. Adipogenic differentiation was carried out for 21 days using adipogenic differentiation medium containing DMEM, 7.5% knockout serum replacement (KOSR; Invitrogen), 7.5% human plasmanate, 0.5% nonessential amino acids, 1% penicillin/streptomycin, 0.1 μM dexamethasone, 10 μg/l insulin (Sigma), and 0.5 μM rosiglitazone. Adipogenic differentiation medium was supplemented for 16 days with doxycyline (700 ng/ml), and afterwards cells were maintained in culture in the absence of doxycycline until day 21 or longer as experiments required.

Derivation and maintenance of adipose-derived stromal vascular cells (ADSVCs). Primary human adipose tissue was obtained from surgical waste of patients who had undergone elective surgery. Adipose was digested with Liberase™ (Roche) for one hour with gentle shaking at 37° C. Digested tissue was forced through a 250 micron filter, and the filtrate was collected and centrifuged. The resulting stromal vascular cell pellet was washed twice with PBS and plated onto gelatin-coated plates (0.1%) in ADSVC growth media [DMEM, 10% FBS, 1% penicillin/streptomycin, and 2.5 ng/ml bFGF (Aldevron)]. ADSVCs were passaged using trypsin upon reaching confluency.

Production of lentivirus and transduction. A third-generation, Tat-free packaging system⁵¹ was used to produce recombinant lentivirus. The vectors—either Lenti-rtTA plasmid^{5, 37, 52, 53} or Lenti-PPARG2 plasmid—together with the two packaging plasmids—pMDL, pREV—and the plasmid coding for VSV-G envelope were transfected into HEK 293T cells using calcium chlorate as previously described⁵⁴. Cells were transduced with lentiviral supernatant 24 hr after passaging at about 40% confluency.

Flow cytometry analysis. Cells were trypsinized, washed with PBS, and centrifuged at 1000 rpm for 5 minutes. Cells were counted and 1×10⁶ cells were transferred into polypropylene tubes. Staining for surface antigens antigen was performed using antibody conjugated to PE, FITC, PE-Cy7 or PE-Cy5 (PE-CD73, Ecto-5′-nucleotidase AD2; BD Pharmingen, PE-CD105 (Endoglin) eBioscience, PE-StrolSanta Cruz, FITC-CD44 eBioscience, PEcy5-CD29 eBioscience, PEcy7-CD4 BD Pharmingen, PE-CD19 eBioscience). Typically 1×10⁶ cells in 80 or 95 μPBS with 5% FBS were stained using 20 μl or 5 μl antibody solution (final volume 100 μl). 10,000 events per cell type were acquired on a FACSCalibur flow cytometer (BD Biosciences). Data were analyzed using the FlowJo software package (Treestar).

Immunocytochemistry. Immunostaining was performed using the following antibodies: α-FABP4 (R&D Systems), α-Perilipin (Sigma), α-CEBPA (Santa Cruz), α-PPARG2 (Santa Cruz), α-MAB1281 (Millipore), α-PRDM16 (Sigma), α-CEBPB (Sigma), α-UCP1 (Sigma), MitoTracker (Invitrogen) and Alexa Fluor secondary antibodies (Invitrogen). Lipid droplets were stained using BODIPY neutral lipid dye. Hoechst or DAPI stain was used to mark cell nuclei. Images were acquired either with a Nikon Digital Sight camera mounted to a Nikon Eclipse Ti-S microscope, an Olympus DP72 camera mounted to a Olympus 1X71 microscope or a Zeiss LSM510 Meta confocal microscope. The NIS-Elements and Olympus DP2-BSW software packages were used for image analysis.

RNA extraction, cDNA synthesis, and quantitative RT-PCR. Total RNA from human cell lines and human fat was extracted with Trizol (Invitrogen) and purified via the RNeasy mini kit (Qiagen) according to the manufacturer's instructions. The RNA yield was determined using the NanoDrop ND-1000 spectrophotometer (NanoDrop Technologies). 1 μg of total RNA was converted to cDNA using the Superscript First-Strand Kit (Invitrogen).

Quantitative RT-PCR was performed using a Realplex Mastercycler (Eppendorf) with the Quantifast-SYBR Green PCR mix (Qiagen) with 1 μl cDNA per reaction. Primer sequences are listed in FIG. 7D.

Transcriptional profiling. Total RNA from human cell lines or human fat was extracted with Trizol (Invitrogen) and purified via the RNeasy mini kit (Qiagen) according to the manufacturers' instructions. RNA quality was assessed using an Aglient Bioanalyzer. RNA probes for microarray hybridization were prepared and hybridized to the Affymetrix GeneChip Human Gene 1.0 ST Microarray, Human Genome U133 Plus 2.0 Array, and Agilent GE G3 arrays.

Lipolysis assay. To measure lipolysis activity, day 21 adipocytes, were incubated with 1-10 μM Isoproterenol or with 10 μM Forskolin. The culture media was collected for glycerol measurement using the Free Glycerol reagent (Sigma #F6428). Protein concentrations used to normalize glycerol content were measured using the Bradford protein assay (BioRad). Glycerol release was expressed in μg glycerol per mg total protein or the lowest value is set to 1.

Adiponectin and Leptin ELISA. Cell culture supernatants were analyzed for adiponectin using the Protein Immunoassay kit (Millipore Corporation) as per the manufacturer's protocol. Cytokine concentrations were calculated using Upstate Beadview software with a five parameter curve-fitting algorithm applied for standard curve calculations.

Metabolomic profiling. All data on extracted lipids was acquired using an Applied Biosystems QSTAR XL hybrid quadrupole/time-of-flight mass spectrometer⁴⁸. MultiQuant software (version 1.1; Applied Biosystems/Sciex) was used for automated peak integration, and peaks were manually reviewed for quality of integration. Internal standard peak areas were monitored for quality control and used to normalize analyte peak areas.

Western blot analysis. Western blot analysis was carried out using following antibodies: total AKT (Cell signaling #9272), phospho-Ser 473 AKT (Cell signaling #9271), Lamin A/C (Fisher/Millipore #3211).

Glucose uptake assay. Adipocytes were serum-starved in 0.2% BSA DMEM overnight. Then, cells were incubated in KRH buffer (121 mM NaCl, 4.9 mM KCl, 1.2 mM MgSO4, 0.33 mM CaCl2, 12 mM HEPES, pH7.4) in the absence or presence of 100 nM insulin for 30 min at 37° C., followed by washing three times in KRH buffer. Glucose uptake was measured by incubating cells with 0.5 μCi/ml 2-deoxy-D-[3H] glucose (Perkin-Elmer) for 5 min at 37° C. After cold PBS washing three times, cells were lysed with 1% Triton X-100 solution and subjected to scintillation counting. Non-specific uptake was measured in the presence of 10 μM of cytochalasin B and subtracted from total uptake.

Transplantation. PPARG2 or PPARG2-CEBPB transduced HUES9 MPC cells were differentiated 14 days. To avoid rejection, differentiated cells were injected subcutaneously into Rag2-/-Il2-/-mice. Four-six weeks after transplantation, mice were sacrificed to collect fat pads. 3T3-F442A cells were injected into the same mice as a positive control. Tissues at the transplantation site were embedded in paraffin and stained after sectioning.

PET analysis. To detect the molecular activity of programmed human white and brown adipose tissue, mice were intravenously injected with [18F] FDG, a glucose analog, two hours prior to PET acquisition. The skin with transplanted human adipose tissue was removed and scanned to visualize the PET signal in the ex vivo sample. PET imaging was performed over two hours using an Inveon small animal scanner (Siemens, D.C.). Osirix was used for visualization of the DICOM images and obtained images were reconstructed using IRM software.

Measurement of cellular OCR and ECAR. Cells were plated in gelatin-coated XF24-well cell culture microplates (Seahorse Bioscience) and differentiated into adipocytes. Cells were incubated in prewarmed unbuffered DMEM medium (DMEM containing 2 mM GlutaMax, 1 mM sodium pyruvate, 1.85 g/l NaCl, and 25 mM glucose) for 1 hour. The oxygen consumption was measured by the XF24 Extracellular Flux Analyzer (Seahorse Biosciences). Mitochondrial biogenesis was profiled by injecting perturbation drugs, 2 μM oligomycin, 0.5 μM CCCP (carbonyl cyanide p-trifluoromethoxyphenylhydrazone), and 5 μM antimycin A in succession. OCR and ECAR were determined by plotting the oxygen tension and acidification of the medium in the chamber as a function of time and normalized by protein concentration (pmol/min/mg and mpH/min/mg), respectively.

### Example 2

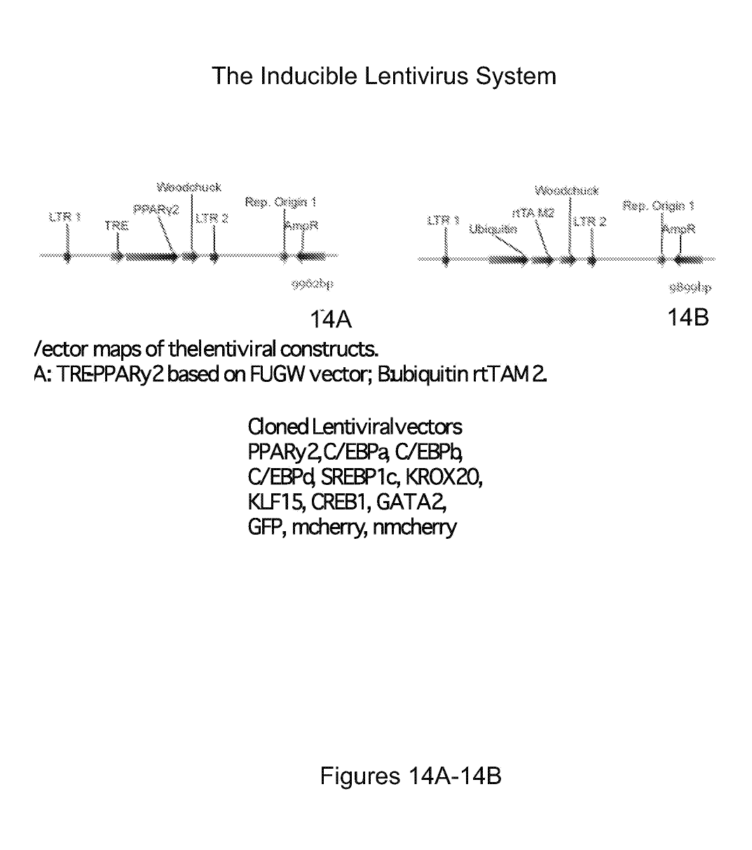

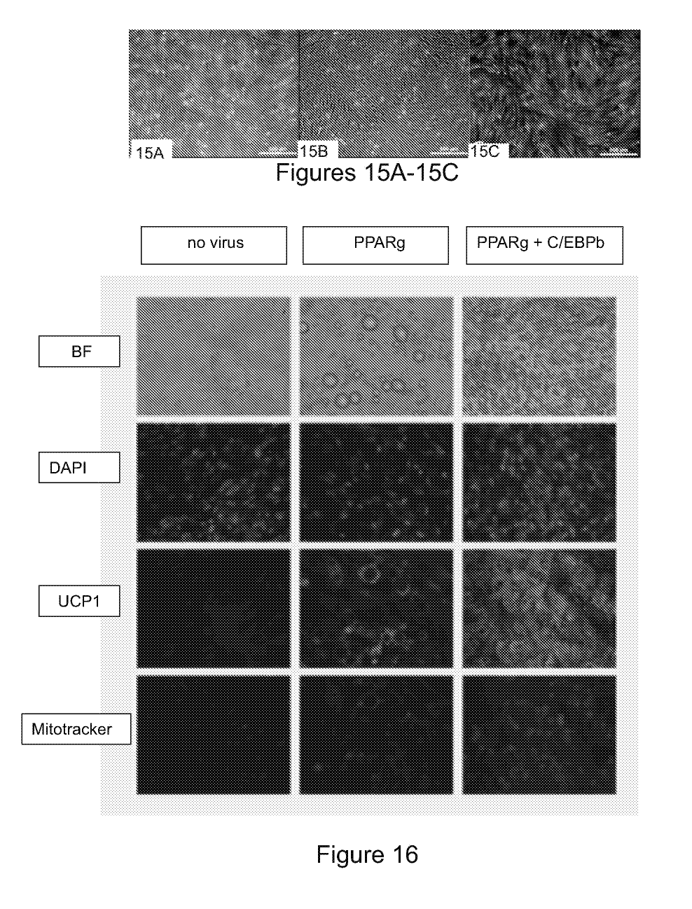

The human PPARγ2, C/EBPB, and PRDM16 genes were cloned into a doxycycline-inducible lentiviral backbone (FIGS. 14A-14B). The adipogenic activity of PPARγ2, C/EBPβ or PRDM16 was demonstrated by ectopically expressing them in human pluripotent cells. The viral transduction (FIGS. 15A-15C) and inducible expression of PPARγ2, C/EBPBβ and PRDM16 factors in human pluripotent cells combined with the addition of insulin, rosiglitazone, dexamethasone, and isobutylmethylxanthine to the cells' growth medium resulted in the appearance of lipid filled cells with multilocular lipid droplets and brown color. In contrast the no virus control showed no visible lipid accumulations. PPARg by itself is the main factor driving white adipose tissue differentiation and was used in our setup as a control to distinguish between white and brown-like adipocytes. The PPARg control showed monocular lipid droplets and no brown coloration of the cells (FIG. 16). Furthermore UCP1 was expressed in those cells only at low levels as expected especially through the addition of rosiglitazone. Importantly the setup with PPARg/C/EBP and PRDM16 showed an increased expression for the mature brown adipocyte markers UCP1. Furthermore Mitotracker staining of the cells indicated a high amount of mitochondria in the cells, another characteristic feature of brown adipose tissue.

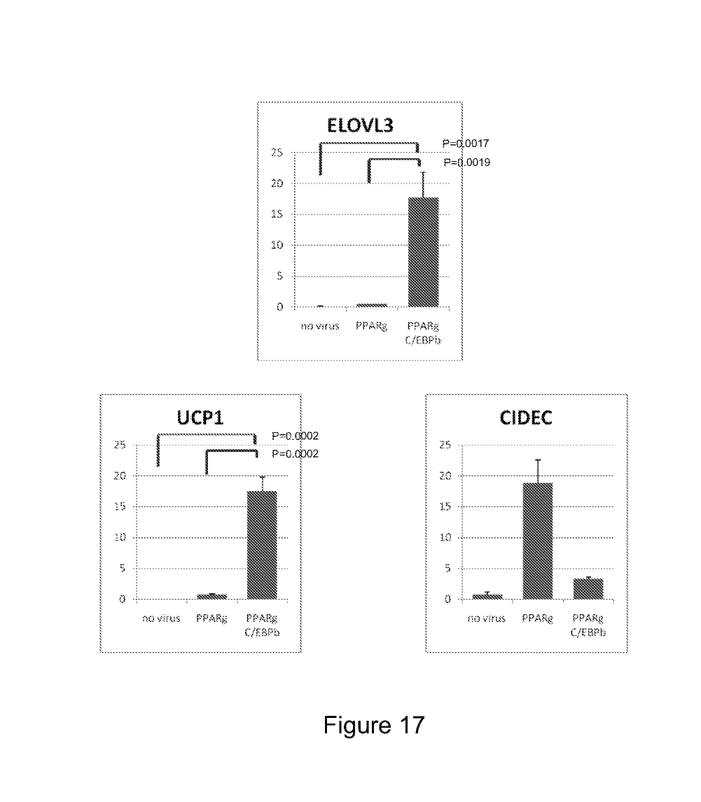

To further characterize these brown-like cells and compare it to the white adipocyte control setup, the expression levels were quantified via reverse transcription real time PCR (FIG. 17). A significant increase of the brown fat markers UCP1 and ELOVL3 was found. As expected the white adipose tissue marker CIDEC was significantly higher expressed in the PPARg control in comparison to the PPARg/C/EBP and PRDM16 setups.

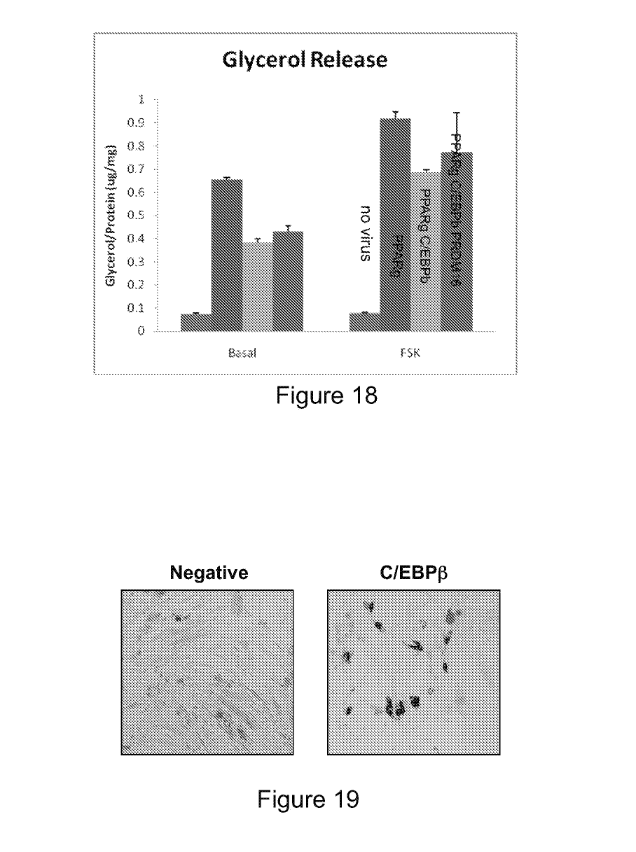

Finally the cells were functionally characterized via Glycerol release after Forskolin induction (FIG. 18). As predicted the cells reacted the Forskolin induced cAMP influx with increased metabolic activity as indicated through Glycerol release. The result was significantly higher in comparison to the no virus control.

### Example 3

### Differentiation of Brown Adipocytes from Fibroblasts

Differentiation of brown adipocytes from fibroblast cells according to the methods described in Example 1 was demonstrated. FIG. 19 depicts the characterization of the brown adipocytes derived from fibroblasts. The brown adipocytes demonstrated the presence of lipids as detected by ORO staining (FIG. 19) and the expression of proteins (e.g CIDEA, UCP1, and FABP4) characteristic of brown adipocytes (data not shown), in contrast to the fibroblasts from which they were derived (FIGS. 19 and data not shown).

### Example 4

### References

1. F. Thomson, J. A. et al. Embryonic stem cell lines derived from human blastocysts. Science 282, 1145-1147 (1998).

2. Takahashi, K. et al. Induction of pluripotent stem cells from adult human fibroblasts by defined factors. Cell 131, 861-872, doi: S0092-8674(07)01471-7[pii] 10.1016/j.cell.2007.11.019 (2007).

3. Yu, J.et al. Induced Pluripotent Stem Cell Lines Derived from Human Somatic Cells. Science 318, 1917-1920, doi:10.1126/science.1151526 (2007).

4. Park, I. H. et al. Reprogramming of human somatic cells to pluripotency with defined factors. Nature 451, 141-146, doi:nature06534 [pii]10.1038/nature06534 (2008).

5. Park, I. H. et al. Disease-specific induced pluripotent stem cells. Cell 134, 877-886, doi:S0092-8674(08)01001-5 [pii] 10.1016/j.cell.2008.07.041 (2008).

6. Dimos, J. T.et al. Induced pluripotent stem cells generated from patients with ALS can be differentiated into motor neurons. Science 321, 1218-1221, doi:1158799 [pii]10.1126/science.1158799 (2008).

7. Lee, G. et al. Modelling pathogenesis and treatment of familial dysautonomia using patient-specific iPSCs. Nature 461, 402-406, doi:nature08320[pii]10.1038/nature08320 (2009).

8. Green, H. & Kehinde, 0. An established preadipose cell line and its differentiation in culture. II Factors affecting the adipose conversion. Cell 5, 19-27 (1975).)

9. Farmer, S. R. Transcriptional control of adipocyte formation. Cell Metab 4, 263-273, doi:S1550-4131(06)00240-3 [pii]10.1016/j.cmet.2006.07.001 (2006).

10. Tontonoz, P., Hu, E., Graves, R. A., Budavari, A. I. & Spiegelman, B. M. mPPAR gamma 2: tissue-specific regulator of an adipocyte enhancer. Genes Dev 8, 1224-1234 (1994).

11. Tontonoz, P., Hu, E. & Spiegelman, B. M. Stimulation of adipogenesis in fibroblasts by PPAR gamma 2, a lipid-activated transcription factor. Cell 79, 1147-1156, doi:0092-8674(94)90006-X [pii] (1994).

12. Cannon, B. & Nedergaard, J. Brown adipose tissue: function and physiological significance. Physiol Rev 84, 277-359, doi:10.1152/physrev.00015.2003 84/1/277[pii] (2004).

13. Cypess, A. M. et al. Identification and importance of brown adipose tissue in adult humans. N Engl J Med 360, 1509-1517, doi:360/15/1509 [pii]10.1056/NEJMoa0810780 (2009).

14. Saito, M. et al. High incidence of metabolically active brown adipose tissue in healthy adult humans: effects of cold exposure and adiposity. Diabetes 58, 1526-1531, doi:db09-0530 [pii] 10.2337/db09-0530 (2009).

15. van Marken Lichtenbelt, W. D. et al. Cold-activated brown adipose tissue in healthy men. N Engl J Med 360, 1500-1508, doi:360/15/1500 [pii]10.1056NEJMoa0808718 (2009).

16. Virtanen, K. A. et al. Functional brown adipose tissue in healthy adults. N Engl J Med 360, 1518-1525, doi:360/15/1518 [pii] 10.1056/NEJMoa0808949 (2009).

17. Seale, P. et al.Transcriptional control of brown fat determination by PRDM16. Cell Metab 6, 38-54, doi:S1550-4131(07)00157-X [pii] 10.1016/j.cmet.2007.06.001 (2007).

18. Seale, P. et al. PRDM16 controls a brown fat/skeletal muscle switch. Nature 454, 961-967, doi:nature07182 [pii] 10.1038/nature07182 (2008).

19. Kajimura, S. et al. Initiation of myoblast to brown fat switch by a PRDM16-C/EBPbeta transcriptional complex. Nature 460, 1154-1158, doi:nature08262 [pii] 10.1038/nature08262 (2009).

20. Pittenger, M. F. et al. Multilineage potential of adult human mesenchymal stem cells. Science 284, 143-147 (1999).

21. Lazarus, H. M., Haynesworth, S. E., Gerson, S. L., Rosenthal, N. S. & Caplan, A. I. Ex vivo expansion and subsequent infusion of human bone marrow-derived stromal progenitor cells (mesenchymal progenitor cells): implications for therapeutic use. Bone Marrow Transplant 22. 16, 557-564 (1995).

23 Zuk, P. A. et al. Multilineage cells from human adipose tissue: implications for cellbased therapies. Tissue Eng 7, 211-228, doi:10.1089/107632701300062859 (2001).

24 Guilak, F., Awad, H. A., Fermor, B., Leddy, H. A. & Gimble, J. M. Adipose-derived adult stem cells for cartilage tissue engineering. Biorheology 41, 389-399 (2004).

25. Mikkelsen, T. S. et al. Comparative epigenomic analysis of murine and human adipogenesis. Cell 143, 156-169, doi:S0092-8674(10)01016-0 [pii] 10.1016/j.cell.2010.09.006 (2010).

26. Barberi, T. & Studer, L. Mesenchymal cells. Methods Enzymol 418, 194-208, doi:S0076-6879(06)18012-X [pii] 10.1016/S0076-6879(06)18012-X (2006).

27. Dani, C. Embryonic stem cell-derived adipogenesis. Cells Tissues Organs 165, 173-180 (1999).

28. Xiong, C. et al. Derivation of adipocytes from human embryonic stem cells. Stem Cells Dev 14, 671-675 (2005).)

29. A van Harmelen, V. et al. Differential lipolytic regulation in human embryonic stem cell-derived adipocytes. Obesity (Silver Spring)15, 846-852 (2007).

30. Wolvetang, E. J. & Hannan, N. Adipocyte differentiation in human embryonic stem cells transduced with Oct4 shRNA lentivirus. Stem Cells Dev (2008).

31. Barberi, T., Willis, L. M., Socci, N. D. & Studer, L. Derivation of multipotent mesenchymal precursors from human embryonic stem cells. PLoS Med 2, e161, doi:04-PLME-RA-0237R2 [pii] 10.1371/journal.pmed.0020161 (2005).

32. Trivedi, P. & Hematti, P. Derivation and immunological characterization of mesenchymal stromal cells from human embryonic stem cells. Exp Hematol 36, 350-359, doi:S0301-472X(07)00642-X [pii] 10.1016/j.exphem.2007.10.007 (2008).

33. Warren, L. et al. Highly efficient reprogramming to pluripotency and directed differentiation of human cells with synthetic modified mRNA. Cell Stem Cell 7, 618-630, doi:S1934-5909(10)00434-0 [pii] 10.1016/j.stem.2010.08.012 (2010).

34. Olivier, E. N., Rybicki, A. C. & Bouhassira, E. E. Differentiation of human embryonic stem cells into bipotent mesenchymal stem cells. Stem cells (Dayton, Oh.)24, 1914-1922, doi:2005-0648 [pii] 10.1634/stemcells.2005-0648 (2006).

35. Tashiro, K. et al.Efficient adenovirus vector-mediated PPAR gamma gene transfer into mouse embryoid bodies promotes adipocyte differentiation. J Gene Med 10, 498-507, doi:10.1002/jgm.1171 (2008).

36. Bermingham, J. R., Jr. et al. Identification of genes that are downregulated in the absence of the POU domain transcription factor pou3f1 (Oct-6, Tst-1, SCIP) in sciatic nerve. J Neurosci 22, 10217-10231, doi:22/23/10217 [pii] (2002).

37. Rosen, E. D. et al. PPAR gamma is required for the differentiation of adipose tissue invivo and in vitro. Mol Cell 4, 611-617, doi:S1097-2765(00)80211-7 [pii] (1999).

38. Maherali, N. et al. A high-efficiency system for the generation and study of human induced pluripotent stem cells. Cell Stem Cell 3, 340-345, doi:S1934-5909(08)00401-3 [pii] 10.1016/j.stem.2008.08.003 (2008).

39. Tang, W. et al. White fat progenitor cells reside in the adipose vasculature. Science 322, 583-586, doi:1156232 [pii] 10.1126/science.1156232 (2008).

40. Altiok, S., Xu, M. & Spiegelman, B. M. PPARgamma induces cell cycle withdrawal: inhibition of E2F/DP DNA-binding activity via down-regulation of PP2A. Genes Dev 11, 1987-1998 (1997).

41. Rosen, E. D. & MacDougald, O. A. Adipocyte differentiation from the inside out. Nat Rev Mol Cell Biol 7, 885-896, doi:nrm2066 [pii] 10.1038/nrm2066 (2006).

12. Svensson, P. A. et al. Gene expression in human brown adipose tissue. Int J Mol Med 27, 227-232, doi:10.3892/ijmm.2010.566 (2011).

43. Tusher, V. G., Tibshirani, R. & Chu, G. Significance analysis of microarrays applied to the ionizing radiation response. Proc Natl Acad Sci U S A 98, 5116-5121, doi:10.1073/pnas.091062498 091062498 [pii] (2001).

44. Puigserver, P. et al. A cold-inducible coactivator of nuclear receptors linked to adaptive thermogenesis. Cell 92, 829-839, doi:S0092-8674(00)81410-5 [pii] (1998).

45. de Jesus, L. A. et al. The type 2 iodothyronine deiodinase is essential for adaptive thermogenesis in brown adipose tissue. J Clin Invest 108, 1379-1385, doi:10.1172/JCI13803 (2001).

46. Zhang, Y. et al.Positional cloning of the mouse obese gene and its human homologue. Nature 372, 425-432, doi:10.1038/372425a0 (1994).

47. Simpson, F. & Whitehead, J. P. Adiponectin—it's all about the modifications. Int j Biochem Cell Biol 42, 785-788, doi:S1357-2725(09)00374-4 [pii] 10.1016/j.biocel.2009.12.021 (2010).

48. Lewis, G. D. et al. Metabolic signatures of exercise in human plasma. Sci Transl Med 2, 33ra37, doi:2/33/33ra37 [pii] 10.1126/scitranslmed.3001006 (2010).

49. Rhee, E. P. et al. Metabolite profiling identifies markers of uremia. J Am Soc Nephrol 21, 1041-1051, doi:ASN.2009111132 [pii] 10.1681/ASN.2009111132 (2010).

50. Jiao, P. et al. FFA-induced adipocyte inflammation and insulin resistance: involvement of ER stress and IKKbeta pathways. Obesity (Silver Spring) 19, 483-491, doi:oby2010200 [pii] 10.1038/oby.2010.200 (2011).

51. Nedergaard, J., Bengtsson, T. & Cannon, B. Unexpected evidence for active brown adipose tissue in adult humans. Am J Physiol Endocrinol Metab 293, E444-452, doi:00691.2006 [pii] 10.1152/ajpendo.00691.2006 (2007).

52. Tiscornia, G., Singer, O. & Verma, I. M. Production and purification of lentiviral vectors. Nat Protoc 1, 241-245, doi:nprot.2006.37 [pii] 10.1038/nprot.2006.37 (2006).

53. Maherali, N. et al. Directly reprogrammed fibroblasts show global epigenetic remodeling and widespread tissue contribution. Cell Stem Cell 1, 55-70, doi:S1934-5909(07)00020-3 [pii] 10.1016/j.stem.2007.05.014 (2007).

54. Stadtfeld, M., Maherali, N., Breault, D. T. & Hochedlinger, K. Defining molecular cornerstones during fibroblast to iPS cell reprogramming in mouse. Cell Stem Cell 2, 230-240 (2008).

55. al Yacoub, N., Romanowska, M., Haritonova, N. & Foerster, J. Optimized production and concentration of lentiviral vectors containing large inserts. J Gene Med 9, 579-584, doi:10.1002/jgm.1052 (2007).

56. Barberi, T., Willis, L. M., Socci, N. D. & Studer, L. Derivation of multipotent mesenchymal precursors from human embryonic stem cells. PLoS Med 2, e161, doi:04-PLME-RA-0237R2[pii] 10.1371/journal.pmed.0020161 (2005).

57. Kopher, R. A. et al. Human embryonic stem cell-derived CD34+cells function as MSC progenitor cells. Bone 47, 718-728, doi:S8756-3282(10)01313-X [pii] 10.1016/j.bone.2010.06.020 (2010).

58. Lian, Q. et al. Derivation of clinically compliant MSCs from CD105+, CD24- differentiated human ESCs. Stem cells (Dayton, Oh.) 25, 425-436, doi:2006-0420 [pii] 10.1634/stemcells.2006-0420 (2007).

59. Lee, G. et al. Isolation and directed differentiation of neural crest stem cells derived from human embryonic stem cells. Nature biotechnology 25, 1468-1475, doi:nbt1365 [pii] 10.1038/nbt1365 (2007).

60. Karnoub, A. E. et al. Mesenchymal stem cells within tumour stroma promote breast cancer metastasis. Nature 449, 557-563, doi:nature06188 [pii] 10.1038/nature06188 (2007).

61. Delorme, B. et al. Specific plasma membrane protein phenotype of culture-amplified and native human bone marrow mesenchymal stem cells. Blood 111, 2631-2635, doi:blood-2007-07-099622 [pii] 10.1182/blood-2007-07-099622 (2008).

62. Wang, F. et al. Transcriptional profiling of human mesenchymal stem cells transduced with reporter genes for imaging. Physiol Genomics 37, 23-34, doi:00300.2007 [pii] 10.1152/physiolgenomics.00300.2007 (2009).

63. Fukuhara, S. et al. Differential function of Tie2 at cell-cell contacts and cell-substratum contacts regulated by angiopoietin-1. Nat Cell Biol 10, 513-526, doi:ncb1714 [pii] 10.1038/ncb1714 (2008).

64. Tusher, V. G., Tibshirani, R. & Chu, G. Significance analysis of microarrays applied to the ionizing radiation response. Proc Natl Acad Sci U S A 98, 5116-5121, doi:10.1073/pnas.091062498 091062498 [pii] (2001).

65. Huang da, W., Sherman, B. T. & Lempicki, R. A. Systematic and integrative analysis of large gene lists using DAVID bioinformatics resources. Nat Protoc 4, 44-57, doi:nprot.2008.211 [pii]10.1038/nprot.2008.211 (2009).

66. Huang da, W., Sherman, B. T. & Lempicki, R. A. Bioinformatics enrichment tools: paths toward the comprehensive functional analysis of large gene lists. Nucleic Acids Res 37, 1-13, doi:gkn923 [ph] 10.1093/nar/gkn923 (2009).

67. Montague, C. T. et al. Depot-related gene expression in human subcutaneous and omental adipocytes. Diabetes 47, 1384-1391 (1998).

68. Gottschling-Zeller, H., Birgel, M., Scriba, D., Blum, W. F. & Hauner, H. Depot-specific release of leptin from subcutaneous and omental adipocytes in suspension culture: effect of tumor necrosis factor-alpha and transforming growth factor-beta1. Eur J Endocrinol 141, 436-442, doi:1410436 [pii] (1999).

69. Zha, J. M. et al. Comparison of gene transcription between subcutaneous and visceral adipose tissue in Chinese adults. Endocr J 56, 935-944, doi:JST.JSTAGE/endocrj/K09E-091 [pii] (2009).

70. Montague, C. T., Prins, J. B., Sanders, L., Digby, J. E. & O'Rahilly, S. Depot- and sex-specific differences in human leptin mRNA expression: implications for the control of regional fat distribution. Diabetes 46, 342-347 (1997).

71. Perrini, S. et al. Fat depot-related differences in gene expression, adiponectin secretion, and insulin action and signalling in human adipocytes differentiated in vitro from precursor stromal cells. Diabetologia 51, 155-164, doi:10.1007s00125-007-0841-7 (2008).

What is claimed herein:

1. A method for promoting the differentiation of cells into brown adipocytes comprising;
a) contacting a population of cells with at least one agent that increases the level or activity of PPARγ2 and C/EBPβ; and
b) culturing the cells under conditions favorable for differentiation into brown adipocytes;
wherein the method does not comprise contacting the cells with an agent which increases the level of PRDM16.

2. The method of claim 1, wherein the agent that increases the level or activity of PPARγ2 and C/EBPβ comprises a polynucleotide comprising a gene sequence that encodes a PPARγ2 and/or a C/EBPβ polypeptide.

3. The method of claim 1, wherein the agent that increases the level or activity of PPARγ2 and C/EBPβ comprises a PPARγ2 polypeptide and/or C/EBPβ polypeptide.

4. The method of claim 1, wherein the agent that increases the level or activity of PPARγ2 and C/EBPβ comprises a small molecule that increases the level or activity of PPARγ2 or C/EBPβ.

5. The method of claim 3, wherein the small molecule is a selected from the group consisting of:
a thiazolidinedione or a glitazar.

6. The method of claim 1, wherein the cells are selected from the group consisting of:
non-neuronal somatic cells, differentiated non-neuronal cells, fibroblasts, adipose-derived cells, adipose-derived stromal vascular cells, and stem or progenitor cells.

7. The method of claim 6, wherein the stem cells or progenitor cells are chosen from the group consisting of:
induced pluripotent stem cells, adipose-derived stem cells, adipose-derived mesenchymal stem cells, adipose progenitor cells, embryonic stem cells, and mesenchymal stem cells.

8. The method of claim 1, wherein said cells are initially provided by inducing a population of pluripotent stem cells to differentiate to a mesenchymal stem cell phenotype.

9. The method of claim 1, wherein the cells are human cells.

10. The method of claim 1, wherein the brown adipocytes are differentiated in vitro.

11. The method of claim 1, wherein the brown adipocytes are differentiated ex vivo.

12. The method of claim 1, wherein the rate of differentiation to brown adipocytes is at least 80%.

13. A method for promoting the differentiation of pluripotent stem cells into brown adipocytes comprising;
differentiating pluripotent stem cells into mesenchymal stem cells;
contacting the mesenchymal stem cells with at least one agent that increases the level or activity of PPARγ2 and C/EBPβ; and
culturing the cells under conditions favorable for the differentiation into brown adipocytes;
wherein the method does not comprise contacting the cells with an agent that increases the level or activity of PRDM16.

14. A method for screening for agents that increase the development of brown adipocytes comprising;
contacting cells with at least one agent that increases the level or activity of PPARγ2 and C/EBPβ;
contacting the cells with an additional candidate agent; and
culturing the cells under conditions favorable for differentiation into brown adipocytes; wherein a candidate agent is identified as an agent that increases the development of brown adipocytes if the rate of proliferation or rate of differentiation of brown adipocytes is higher in the presence of the candidate agent.

15. A method for screening for agents that increase the activity of brown adipocytes comprising;
contacting cells with at least one agent that increases the level or activity of PPARγ2 and C/EBPβ;
culturing the cells under conditions favorable for differentiation into brown adipocytes; and
contacting the brown adipocytes with a candidate agent;
wherein a candidate agent is identified as an agent that increases the activity of brown adipocytes if a measure of brown adipocyte activity is higher in the presence of the candidate agent.

16. The method of claim 15, wherein the measure of brown adipocyte activity is the generation of heat.

17. The method of claim 15, wherein the measure of brown adipocyte activity is the rate of growth or proliferation of the adipocytes.

18. The method of claim 15, wherein the measure of brown adipocyte activity is selected from the group consisting of:
expression of brown adipocyte marker genes; measurement of mitochondrial number and activity; and glycerol release.

19. A method of providing brown adipocytes to a subject in need thereof comprising;
differentiating brown adipocytes from cells ex vivo according to the method of claim 1; and
transplanting the brown adipocytes so differentiated into the subject.

20. The method of claim 19, wherein the cells are autologous.

21. (canceled)

22. (canceled)

23. (canceled)
